(19) United States
(12) Patent Application Publication
(10) Pub. No.: US 2023/0303983 A1
(43) Pub. Date: Sep. 28, 2023

(54) HIGH EFFICIENCY PURIFICATION OF DIVERGENT AAV SEROTYPES USING AAVX AFFINITY CHROMATOGRAPHY

(71) Applicants: President and Fellows of Harvard College, Cambridge, MA (US); Massachusetts Eye and Ear Infirmary, Boston, MA (US)

(72) Inventors: Michael Florea, Cambridge, MA (US); Amy J. Wagers, Cambridge, MA (US); Luk Vandenberghe, Weston, MA (US)

(21) Appl. No.: 17/743,444

(22) Filed: May 12, 2022

Related U.S. Application Data
(60) Provisional application No. 63/324,618, filed on Mar. 28, 2022.

(51) Int. Cl. C12N 7/02; B01D 15/36; C12N 15/864

(52) U.S. Cl. CPC C12N 7/02; B01D 15/361; C12N 15/8645; C12N 2750/14143; C12N 2750/14151

(57) ABSTRACT

Disclosed herein are optimized methods of high efficiency purification of adeno-associated virus (AAV) particles, comprising a step of binding one or more AAV particles with a volume of chromatography resin medium comprising at least one ligand possessing a pan-AAV affinity. The optimized methods are capable of providing purified AAV particles comprising a wide diversity of AAV serotypes without the need to further optimize for a given AAV serotype.

### RELATED APPLICATION

This application claims the benefit of U.S. Provisional Application No. 63/324,618, filed on Mar. 28, 2022. The entire teachings of the above application(s) are incorporated herein by reference.

### GOVERNMENT SUPPORT

This invention was made with government support under grant awarded by National Institutes of Health. The government has certain rights in the invention.

### SEQUENCE LISTING

Sequence Listing associated with this application is provided in text format in lieu of a paper copy and is hereby incorporated by reference into the specification. The name of the text file containing the Sequence Listing is HRVY_203_101_seqlist.txt. The text file is 56.7 KB, was created on Aug. 15, 2022, and is being submitted electronically via EFS-Web, concurrent with the filing of the specification.

### BACKGROUND OF THE INVENTION

Adeno-associated viruses (AAVs) are small, non-enveloped single-stranded DNA viruses discovered in 1960s as contaminants of adenovirus preps^{1,2}. They induce limited host response and are not associated with any known disease, yet were found to be highly efficient at delivering DNA cargo to many tissues in multiple animal species³. AAVs are thus widely used as a gene transfer tool in basic research and in translational and clinical gene therapy⁴. Their increased use has increased demand on AAV manufacturing both in terms of the quality of the preparation and the quantity of the material.

Currently, for research and for some clinical purposes, AAV purification often relies on an ultracentrifugation step on an iodixanol or cesium chloride gradient^{5,6}. This process is appealing for two reasons; one, it is serotype agnostic and little process optimization is needed for the various AAV products researchers seek to purify, and second, it remains one of the more efficient methods of separation of genome-containing (or ‘full’) capsids from empty or partially filled capsids. The process step however is difficult to scale, requires precise manual handling and may co-purify any contaminants that have the same sedimentation co-efficient as AAV⁷.

Liquid chromatography provides a more scalable, less laborious, and possibly more efficient purification method, particularly under high-performance liquid chromatography (HPLC) conditions as has been shown for the purification of proteins and small molecules⁸. For AAV, several chromatographic methods have been developed, most using AVB Sepharose affinity, cation exchange or anion exchange chromatography⁹⁻¹². While these methods demonstrate the feasibility and efficiency of chromatographic purification of AAV, they also require substantial serotype-specific optimization and are thus not optimal for purification in a research setting, where many different serotypes need to be purified for different applications.

Recently, several AAV-binding resins have been commercially released, including AVB Sepharose High Performance (GE Healthcare) and POROS CaptureSelect AAV8, AAV9 and AAVX (Thermo Scientific). In the case of AVB, it was recently shown that affinity chromatography using AVB resin can efficiently purify AAV1, AAV2, AAV5, AAV6 and rh10 but requires serotype-specific optimization and fails to purify multiple other serotypes, including the broadly used AAV8 and AAV9 serotypes^{12,13}. POROS CaptureSelect AAV8 and AAV9 resins bind and are recommended for purification of AAV8 and AAV9 respectively, but they are not compatible with other serotypes (POROS CaptureSelect product datasheet)^{10,12}. POROS Captureselect AAVX is a resin consisting of a rigid 50 μm diameter crosslinked poly[styrene divinylbenzene] bead backbone, coated with cross-linked polyhydroxylated polymer, and linked to a camelid heavy-chain-only single-domain antibody fragment. The camelid antibody was raised against a conserved region of the AAV capsid, and the AAVX resin is marketed as a pan-AAV affinity resin capable of binding multiple different AAV serotypes (POROS CaptureSelect product datasheet)¹⁰. Nonetheless, there is still a need to develop a new single optimized process for highly efficient purification of a panel of highly divergent AAVs, including new engineered AAVs, which demonstrates an overall purification efficiency higher than other described methods, while still being simple to execute, low-cost, and minimally laborious.

### SUMMARY OF THE INVENTION

The adeno-associated viral vector (AAV) provides a safe and efficient gene therapy platform with a number of approved products with marked therapeutic impact for patients. However, a major bottleneck in its development and commercialization remains the efficiency, cost, and scalability of AAV production. Chromatographic methods have the potential to allow purification at increased scales and lower cost but often require optimization specific to each serotype. It was recently discovered that by combining multiple innovations into a single optimized chromatography purification process, a panel of highly divergent AAVs could be purified with an overall efficiency higher than other described methods. Furthermore, AAVs purified utilizing this highly efficient, optimized chromatography purification process resulted in purified AAVs which demonstrate similar in vitro and in vivo bioactivity to AAVs purified using ultracentrifugation-based processes.

Disclosed herein, are methods of high efficiency purification of adeno-associated virus (AAV) particles comprising the steps of a) providing a cell lysate that comprises one or more AAV particles; b) contacting the cell lysate comprising the one or more AAV particles with a volume of chromatography resin medium comprising at least one ligand possessing a pan-AAV affinity, wherein said contacting induces binding of the one or more AAV particles to the chromatography resin medium and wherein the contacting is performed at a temperature of between approximately 20° C. to approximately 28° C. and/or both the cell lysate and the chromatography resin medium are allowed to come to a temperature between approximately 20° C. to approximately 28° C. previous to said contacting; and c) eluting the bound one or more AAV particles from the chromatography resin medium using a volume of an acidic elution buffer solution to provide one or more eluted volume fractions containing the one or more AAV particles; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, and wherein the method does not require modifications contingent on the AAV serotype being purified to obtain said efficiency.

In some embodiments, the contacting is performed at a temperature of between approximately 22° C. to 26° C. In some embodiments, the contacting is performed at a temperature of approximately 24° C. or approximately 21° C.

In some embodiments, both the cell lysate and the chromatography resin medium are allowed to come to a temperature between approximately 22° C. to approximately 26° C. prior to the contacting step. In some embodiments, both the cell lysate and the chromatography resin medium are allowed to come to a temperature of approximately 24° C. or approximately 21° prior to the contacting steps.

In some embodiments, the contacting is performed at a temperature of between approximately 22° C. to approximately 26° C. and both the cell lysate and the chromatography resin medium are allowed to come to a temperature between approximately 22° C. to approximately 26° C. prior to the contacting step. In some embodiments, the contacting is performed at a temperature of approximately 24° C. or approximately 21° C. and both the cell lysate and the chromatography resin medium are allowed to come to a temperature of approximately 24° C. or approximately 21° C. prior to the contacting step.

Also disclosed herein are methods of high efficiency purification of adeno-associated virus (AAV) particles comprising the steps of a) providing a cell lysate that comprises one or more AAV particles; b) contacting the cell lysate comprising the one or more AAV particles with a volume of chromatography resin medium comprising at least one ligand possessing a pan-AAV affinity, wherein said contacting induces binding of the one or more AAV particles to the chromatography resin medium; and c) eluting the bound one or more AAV particles from the chromatography resin medium using a volume of an acidic elution buffer solution to provide one or more eluted volume fractions containing the one or more AAV particles; whereby purified AAV particles are obtained with an overall efficiency of at least 65% regardless of the serotype of the AAV purified, wherein at least one of the method steps is carried out in the presence of a nonionic surfactant, wherein the purity, yield and bioactivity of the AAV particles purified thereby are comparable to the purity, yield and bioactivity of iodixanol purified AAV particles, wherein the method does not require modifications contingent on the AAV serotype being purified to obtain said efficiency.

In some embodiments, two or more steps of the method are carried out in the presence of the non-ionic surfactant. In other embodiments, at least three steps of the method are carried out in the presence of the non-ionic surfactant. In some embodiments, the non-ionic surfactant is a tri-block poly(ethylene oxide) (PEO)-poly(propylene oxide) (PPO)— poly(ethylene oxide) (PEO) copolymer. In some embodiments, the non-ionic surfactant is PLURONIC F-68.

Also disclosed herein are methods of high efficiency purification of adeno-associated virus (AAV) particles comprising the steps of a) providing a cell lysate that comprises one or more AAV particles; b) contacting the cell lysate comprising the one or more AAV particles with a volume of chromatography resin medium comprising at least one ligand possessing a pan-AAV affinity, wherein said contacting induces binding of the one or more AAV particles to the chromatography resin medium; c) eluting the bound one or more AAV particles from the chromatography resin medium using a volume of an acidic elution buffer solution to provide one or more eluted volume fractions containing the one or more AAV particles; and d) regenerating the chromatography resin medium comprising the at least one ligand possessing a pan-AAV affinity by contacting the medium with at least one acidic component, wherein contact with the at least one acidic component is carried out for a predetermined amount of time, whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, and wherein the method does not require modifications contingent on the AAV serotype being purified to obtain said efficiency.

In some embodiments, the at least one acidic component comprises phosphoric acid and/or guanidine HCL. In some embodiments, the at least one acidic component comprises two acidic components which are each contacted with the chromatography resin concurrently or sequentially, and wherein the contact with each of the two acidic components is carried out for predetermined amounts of time. In some embodiments, the predetermined amount of time is 30 seconds to 24 hours, 1 minute to 12 hours, 5 minutes to 4 hours, 10 minutes to 1 hour, or 15 minutes to 30 minutes.

In some embodiments, each predetermined amount of time is at least 15 minutes. In some embodiments, the regeneration step is carried out by contacting the chromatography resin medium with 0.1M phosphoric acid for at least 15 minutes, followed or preceded by contacting the chromatography resin medium with 6M guanidine for at least 15 minutes. In some embodiments, the step of regeneration of the chromatography resin medium is performed a second time, a third time, a fourth time, a fifth time, a sixth time, a seventh time, an eighth time, a ninth time or ten or more times.

In some embodiments, the method comprises at least one repetition of steps a) through c). In some embodiments, the completion or repetition of the step of regeneration of the chromatography resin medium results in one or both of i) sustaining high efficiency purification during subsequent repetitions of steps a) through c) and ii) eliminating carry over contamination between repetitions of steps a) through c).

In some embodiments, the one or more AAV particles subjected to purification is selected from one or more AAV serotypes from the group consisting of AAV1, AAV2, AAV2-7m8, AAV-HSPG, AAV3, AAV4, AAV5, AAV6, AAV6.2, AAV7, AAV8, AAV9, Rh10, Rh61, Rh8, Rh32.33, VR-865, PHP.B and Anc80.

In some embodiments, the volume of acidic elution buffer solution comprises glycine, phosphoric acid or citric acid. In some embodiments, the volume of acidic elution buffer solution comprises glycine. In some embodiments, the pH of the acidic elution buffer solution is approximately 3.0 or less. In some embodiments, the pH of the acidic elution buffer solution is between about 2 to about 2.5.

In some embodiments, the chromatography resin medium comprising a ligand possessing a pan-AAV affinity comprises a cross-linked poly(styrene-divinylbenzene) bead coated with a cross-linked polyhydroxylated polymer. In some embodiments, the chromatography resin medium is linked to a ligand which comprises a camelid heavy-chain-only single domain antibody fragment. In some embodiments, the camelid antibody was raised against a conserved region of an AAV capsid. In some embodiments, the chromatography resin medium comprises one or more beads with a diameter of approximately 50 μm.

In some embodiments, the one or more AAV particles comprise a capsid which encapsulates vector DNA or is empty. In some embodiments, the method further comprises a downstream step of performing size exclusion or anion exchange chromatography on the purified one or more AAV particles thereby enriching the AAV particles which encapsulate said vector DNA or performing one or more upstream steps including one or more optimizing plasmid transfection ratios; utilizing vector plasmids that are full length or have minimal ITR deletion; using novel engineered ITRs; and using a transfection plasmid containing both the AAV cap and transgene in cis.

In some embodiments, the method further comprises a step of neutralizing the one or more eluted fractions containing the one or more AAV particles. In some embodiments, the step of neutralizing comprises addition of tris(hydroxymethyl)aminomethane hydrochloride (TRIS-HCL) to the one or more elution fractions.

In some embodiments, the method further comprises one or more washing steps prior to, after, or both prior to and after the elution step. In some embodiments, the one or more washing steps are carried out with a washing buffer solution comprising one or more components selected from the group consisting of tris-buffered saline (TB S), ethanol, guanidine HCL, phosphoric acid, glycine, Tris NaOH, water, a non-ionic surfactant, and NaCl.

In some embodiments, the chromatography resin medium linked to at least one ligand which possess pan-AAV affinity is packed into a column to provide a high performance liquid chromatography column. In some embodiments, the chromatography resin medium linked to at least one ligand which possess pan-AAV affinity is purchased pre-packed into a column. the chromatography resin medium linked to at least one ligand which possess pan-AAV affinity wherein the volume of chromatography resin medium is at least 0.1 mL, at least 0.5 mL, at least 1 mL, at least 2 mL, at least 3 mL, at least 4 mL, at least 5 mL, or at least 10 mL.

In some embodiments, a flow rate of one or both of the acidic elution buffer solution and the wash buffer solution through the chromatography resin medium during the elution or washing steps comprises a flow rate of no more than 5 mL/min, no more than 4 mL/min, no more than 3 mL/min, no more than 2 mL/min, no more than 1 mL/min, no more than 0.5 ml/min or no more than 0.1 ml/min. In some embodiments, a flow rate of one or both of the acidic elution buffer solution and the wash buffer solution through the chromatography resin medium during the elution or washing steps comprises a flow rate per minute of approximately an equal volume or less of the acidic elution buffer solution or wash buffer solution per volume of chromatography resin medium. In some embodiments, a flow rate of one or both of the acidic elution buffer solution and wash buffer solution is 0.5 mL/min to 1 mL/min. In some embodiments, the volume of the chromatography resin medium is approximately 1 mL. In some embodiments, the flow rate of one or both of the acidic elution buffer solution and wash buffer solution is approximately 1 mL/min. In some embodiments, the volume of the chromatography resin medium is approximately 1 mL.

In other embodiments, a direction of flow of the elution acidic buffer solution in an elution step is opposite to a direction of flow of the cell lysate through the chromatography resin medium in the contacting step, whereby an increased quantity of AAV particles are eluted from the chromatography resin medium compared to when the directions of flow in the elution and providing steps are the same.

In some embodiments, the method further comprises a step of capturing the at least one or more elution volume fractions. In some embodiments, the method further comprises a step of sterilizing the at least one or more elution volume fractions containing the purified one or more AAV vector particles. In some embodiments, the method further comprises the step of submitting the at least one or more elution volume fractions to a buffer exchange. In some embodiments, the buffer exchange occurs in an AMICON Stirred Cell concentrator or an AMICON Ultra-15 filter concentrator.

Also disclosed herein are methods of high efficiency purification of adeno-associated virus (AAV) particles comprising the steps of a) transfecting at least one cell with plasmid DNA comprising an AAV vector genome, one or more AAV capsid genes and one or more transacting helper genes; b) providing a cell lysate that comprises one or more AAV particles by lysing the transfected at least one cell in situ; c) contacting the cell lysate comprising the one or more AAV particles with a volume of chromatography resin medium comprising at least one ligand possessing a pan-AAV affinity, wherein said contacting induces binding of the one or more AAV particles to the chromatography resin medium; and d) eluting the bound one or more AAV particles from the chromatography resin medium using a volume of an acidic elution buffer solution to provide one or more eluted volume fractions containing the one or more AAV particles; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, wherein the method does not require modifications contingent on the AAV serotype being purified to obtain said efficiency and wherein at least one of the following is true: i) at least one of the method steps is carried out in the presence of a non ionic surfactant, ii) the contacting is performed at a temperature of between approximately 20° C. to approximately 28° C., iii) both the cell lysate and the chromatography resin medium are allowed to come to a temperature between approximately 20° C. to approximately 28° C. previous to said contacting, or iv) the method further comprises a step of regenerating the chromatography resin medium comprising the at least one ligand possessing a pan-AAV affinity by contacting the medium with at least one acidic component for a predetermined amount of time.

In some embodiments, the contacting is performed at a temperature of between approximately 22° C. to 26° C. In some embodiments, both the cell lysate and the chromatography resin medium are allowed to come to a temperature between approximately 22° C. to approximately 26° C. prior to the contacting step. In some embodiments, the contacting is performed at a temperature of approximately 24° C. or approximately 21° C. In some embodiments, both the cell lysate and the chromatography resin medium are allowed to come to a temperature of approximately 24° C. or approximately 21° prior to the contacting steps. In some embodiments, the contacting is performed at a temperature of between approximately 22° C. to approximately 26° C. and both the cell lysate and the chromatography resin medium are allowed to come to a temperature between approximately 22° C. to approximately 26° C. prior to the contacting step. In some embodiments, the contacting is performed at a temperature of approximately 24° C. or approximately 21° C. and both the cell lysate and the chromatography resin medium are allowed to come to a temperature of approximately 24° C. or approximately 21° C. prior to the contacting step. In some embodiments, the one or more AAV particles subjected to purification is selected from one or more AAV serotypes from the group consisting of AAV1, AAV2, AAV2-7m8, AAV-HSPG, AAV3, AAV4, AAV5, AAV6, AAV6.2, AAV7, AAV8, AAV9, Rh10, Rh61, Rh8, Rh32.33, VR-865, PHP.B and Anc80.

In some embodiments, the volume of acidic elution buffer solution comprises glycine, phosphoric acid or citric acid. In some embodiments, the volume of acidic elution buffer solution comprises glycine. In some embodiments, the pH of the acidic elution buffer solution is approximately 3.0 or less. In some embodiments, the pH of the acidic elution buffer solution is between about 2 to about 2.5.

In some embodiments, the chromatography resin medium comprising a ligand possessing a pan-AAV affinity comprises a cross-linked poly(styrene-divinylbenzene) bead coated with a cross-linked polyhydroxylated polymer. In some embodiments, the chromatography resin medium is linked to a ligand which comprises a camelid heavy-chain-only single domain antibody fragment. In some embodiments, the camelid antibody was raised against a conserved region of an AAV capsid. In some embodiments, the chromatography resin medium comprises one or more beads with a diameter of approximately 50 μm.

In some embodiments, the one or more AAV particles comprise a capsid which encapsulates vector DNA or which is empty. In some embodiments, the method further comprises one or more steps selected from a downstream step of performing size exclusion or anion exchange chromatography on the purified one or more AAV particles thereby enriching the AAV particles which encapsulate said vector DNA and performing one or more upstream steps to reduce AAV particles comprising an empty capsid including optimizing plasmid transfection ratios, utilizing vector plasmids that are full length or have minimal ITR deletion, using novel engineered ITRs, and using a transfection plasmid containing both the AAV cap and transgene in cis.

In some embodiments, two or more steps of the method are carried out in the presence of a non-ionic surfactant. In some embodiments, the non-ionic surfactant is a tri-block poly(ethylene oxide) (PEO)-poly(propylene oxide) (PPO)-poly(ethylene oxide) (PEO) copolymer. In some embodiments, the non-ionic surfactant is PLURONIC F-68. In some embodiments, the method further comprises a step of neutralizing the one or more eluted fractions containing the one or more AAV particles. In some embodiments, the step of neutralizing comprises addition of tris(hydroxymethyl)aminomethane hydrochloride (TRIS-HCL) to the one or more elution fractions. In some embodiments, the method further comprises one or more washing steps prior to, after, or both prior to and after the elution step. In some embodiments, the one or more washing steps are carried out with a washing buffer solution comprising one or more components selected from the group consisting of tris-buffered saline (TBS), ethanol, guanidine HCL, phosphoric acid, glycine, Tris NaOH, water, a non-ionic surfactant, and NaCl.

In some embodiments, the method further comprises a step of regenerating the chromatography resin medium comprising the at least one ligand possessing a pan-AAV affinity by contacting the medium with at least one acidic component, wherein contact with the at least one acidic component is carried out for a predetermined amount of time. In some embodiments, the at least one acidic component comprises phosphoric acid and/or guanidine HCL. In some embodiments, the at least one acidic component comprises two acidic components which are each contacted with the chromatography resin concurrently or sequentially, and wherein the contact with each of the two acidic components is carried out for predetermined amounts of time. In some embodiments, the predetermined amount of time is 30 seconds to 24 hours, 1 minute to 12 hours, 5 minutes to 4 hours, 10 minutes to 1 hour, or 15 minutes to 30 minutes. In some embodiments, each predetermined amount of time is at least 15 minutes. In some embodiments, the regeneration step is carried out by contacting the chromatography resin medium with 0.1M phosphoric acid for at least 15 minutes, followed or preceded by contacting the chromatography resin medium with 6M guanidine for at least 15 minutes. In some embodiments, the step of regeneration of the chromatography resin medium is performed a second time, a third time, a fourth time, a fifth time, a sixth time, a seventh time, an eighth time, a ninth time or ten or more times.

In some embodiments, the method comprises at least one repetition of steps a) through c). In some embodiments, the completion or repetition of the step of regeneration of the chromatography resin medium results in one or both of i) sustaining high efficiency purification during subsequent repetitions of steps a) through c) and ii) eliminating carry over contamination between repetitions of steps a) through c).

The method according to any one of claims 138-171, wherein the chromatography resin medium linked to at least one ligand which possess pan-AAV affinity is packed into a column to provide a high performance liquid chromatography column. In some embodiments, the chromatography resin medium linked to at least one ligand which possess pan-AAV affinity is purchased pre-packed into a column. the chromatography resin medium linked to at least one ligand which possess pan-AAV affinity wherein the volume of chromatography resin medium is at least 0.1 mL, at least 0.5 mL, at least 1 mL, at least 2 mL, at least 3 mL, at least 4 mL, at least 5 mL, or at least 10 mL.

In some embodiments, a flow rate of one or both of the acidic elution buffer solution and the wash buffer solution through the chromatography resin medium during the elution or washing steps comprises a flow rate of no more than 5 mL/min, no more than 4 mL/min, no more than 3 mL/min, no more than 2 mL/min, no more than 1 mL/min, no more than 0.5 ml/min or no more than 0.1 ml/min. In some embodiments, a flow rate of one or both of the acidic elution buffer solution and the wash buffer solution through the chromatography resin medium during the elution or washing steps comprises a flow rate per minute of approximately an equal volume or less of the acidic elution buffer solution or wash buffer solution per volume of chromatography resin medium. In some embodiments, a flow rate of one or both of the acidic elution buffer solution and wash buffer solution is 0.5 mL/min to 1 mL/min. In some embodiments, the volume of the chromatography resin medium is approximately 1 mL. In some embodiments, the flow rate of one or both of the acidic elution buffer solution and wash buffer solution is approximately 1 mL/min. In some embodiments, the volume of the chromatography resin medium is approximately 1 mL.

In some embodiments, a direction of flow of the elution acidic buffer solution in an elution step is opposite to a direction of flow of the cell lysate through the chromatography resin medium in the contacting step, whereby an increased quantity of AAV particles are eluted from the chromatography resin medium compared to when the directions of flow in the elution and providing steps are the same.

In some embodiments, the method further comprises a step of capturing the at least one or more elution volume fractions. In some embodiments, the method further comprises a step of sterilizing the at least one or more elution volume fractions containing the purified one or more AAV vector particles.

In some embodiments, the method further comprises the step of submitting the at least one or more elution volume fractions to a buffer exchange. In some embodiments, the buffer exchange occurs in an AMICON Stirred Cell concentrator or an AMICON Ultra-15 filter concentrator.

Also disclosed herein are particularly preferred methods of high efficiency purification of adeno-associated virus (AAV) particles comprising the steps of: a) seeding HEK293T cells onto a substrate which holds a Dulbecco's modified eagle medium (DMEM) containing 10% fetal bovine serum (FBS) and 1% mixture of penicillin G and streptomycin (penstrep) and allowing expansion of the cells until approximately 80% confluency is obtained; b) transfecting the HEK293T cells by contacting said cells with a composition that comprises DMEM, polyethylenimine, penstrep, and plasmid DNA comprising an AAV vector genome, one or more AAV capsid genes and one or more trans-acting helper genes; c) providing a clarified cell lysate that comprises one or more AAV particles by lysing the transfected HEK293T cells in situ utilizing TRITON-X 100, RNAse A, Turbonuclease and PLURONIC F68, subjecting the lysate to centrifugation at 4,000 g or higher and subsequently filtering a supernatant thus obtained by use of a 0.45 μm cellulose acetate/polyethersulfone membrane filter system; d) contacting the clarified cell lysate comprising the one or more AAV particles with a 1 mL volume of POROS CAPTURESELECT AAVX chromatography resin medium, wherein the contacting duration comprises no less than 1 minute, wherein the contacting is performed at a temperature of between approximately 21° C. to approximately 25° C. and/or both the cell lysate and the chromatography resin medium are allowed to come to a temperature between approximately 21° C. to approximately 25° C. previous to said contacting, and wherein said contacting induces binding of the one or more AAV particles to the chromatography resin medium; e) eluting the bound one or more AAV particles from the chromatography resin medium using a volume of a filtered acidic elution buffer solution having a pH between approximately 2.0 and approximately 2.5, to provide one or more eluted volume fractions containing the one or more AAV particles, wherein the filtered acidic elution buffer solution comprises 0.2M glycine and 0.01 v/v % PLURONIC F68 and wherein a flow direction of the acidic elution buffer solution through the chromatography resin medium is opposite a flow direction of the clarified cell lysate through the chromatography resin during the contacting step; f) optionally sterilizing the at least one or more elution volume fractions containing the purified one or more AAV particles utilizing a 0.2 μm polyethersulfone syringe filter; g) subjecting the one or more elution volume fractions containing the purified one or more AAV particles to buffer exchange using either AMICON UTRACEL 15 or AMICON Stirred Cell concentrators, wherein if a 50 or 100 kDA AMICON ULTRA 15 Centrifugal Filter device is used for buffer exchange, less than 1×10¹³ vg of AAV are subjected to buffer exchange therein to reduce sedimentation and loss; and h) regenerating the chromatography resin medium by contacting the chromatography resin medium with 0.1M phosphoric acid for at least 15 minutes, followed or preceded by contacting the chromatography resin medium with 6M guanidine for at least 15 minutes; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, wherein any plastic surface which comes into contact with the AAV particles during the method is first coated with a composition comprising PLURONIC F68, and wherein the method does not require modifications contingent on the AAV serotype being purified to obtain said efficiency.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E depict AAV purification using AAVX affinity chromatography. FIG. 1A depicts affinity of AAVX to various AAV serotypes tested in a static binding assay. The flow through (FT), wash (W) and eluted fractions (E) were collected and analyzed by qPCR to quantify their vector genome copies. Data represented as percent vector genomes (vg) of the input. Each serotype was applied to unused AAVX resin. FIG. 1B shows phylogeny depicting the diversity of AAV capsids included in this report (bold) along with the percent identity (by amino acid) compared with AAV9. The tree is drawn to scale with branch lengths depicting substitutions per site. VR-865—an Avian AAV used as an outgroup. FIG. 1C depicts AAV purification of AAV2 and Anc80 using AAVX resin in an HPLC setting. Fractions were taken from input, flow-through, at TBS and ethanol wash steps and elution, and AAV content quantified using qPCR. Percent recovery for these purifications is shown above elution bars. FIG. 1D Average purification efficiencies of AAV2 and Anc80 (percent recovery of AAV in the elution). FIG. 1E Total yields of purified AAV2, Anc80, AAV9 and PHP.eB preps across various vectors with no optimization of the process. Each dot represents an AAV prep from one hyperflask (1720 cm² growth area). All purifications were carried out at room temperature, using 1 ml AAVX column at 1 ml/min flow rate. All values estimated are above qPCR limit of detection (approximately 10⁵ vg/ml).

FIGS. 2A-2E depicts the effect of resin regeneration and temperature on purification efficiency. FIG. 2A provides an overview of experimental design of FIGS. 2B and 2C. Five small-scale (one and a half 15 cm dishes per prep) AAV1 preps were produced and purified sequentially on HPLC with AAVX resin, without changing the resin between purifications. Preps two to five were identical except for a 100 bp barcode region. Vector genomes were quantified across all purifications. For the 5th prep, the barcode region was PCR amplified, next-generation sequenced and the unique barcodes corresponding to each prep counted to estimate carry-over contamination from preps 2 to 4. AAV was applied to a 1 ml AAVX column at 1 ml/min flow rate, room temperature. FIG. 2B depicts purification efficiency with repeated resin use. Vector genomes in lysate, flow through and elution. #—some sample was lost due to handling error. FIG. 2C depicts carry-over contamination. Barcode counts from preps 2 to 5, in the 5th prep estimated via NGS. FIG. 2D depicts the effect of purification temperature on the percent of vector genomes found in flow-through for AAV9 and PHP.eB. Difference was assessed using two-way ANOVA with Šídák's post-hoc tests. FIG. 2E depicts the stability of AAV (PHP.eB) in clarified lysate at 24° C. over 96 hours. ** p<0.01, *** p<0.001, **** p<0.0001

FIGS. 3A-3E depicts an optimized AAVX affinity purification process. FIG. 3A depicts process steps of the protocol. FIG. 3B depicts step-wise recovery at each step of the purification process. Vector genomes were quantified via qPCR from aliquots of the sample at each process step and represented as normalized to the lysate. N=6 biological replicates for both AAV9 and PHP.eB. FIG. 3C depicts recovery after filtration+buffer exchange steps for AAV9 and PHP.eB. Note that the values above 100% fall within the range of the approximately 20% precision limit of qPCR titration, and likely do not represent actual recoveries above 100%. FIG. 3D depicts overall purification efficiencies of the non-optimized and optimized protocols for AAV9 and PHP.eB combined. Difference was assessed using a two-tailed t-test, with *=p<0.05. FIG. 3E depicts total yields per hyperflask across all vectors produced with scAAV9 and scPHP.eB and purified using this protocol. Note that this includes some vectors that have lower-than average production yields. Detailed steps of the purification process are listed in Supplementary Protocol 1.

FIGS. 4A-4C depicts quality and in vitro bioactivity of AAVX affinity purified AAV. FIG. 4A depicts SYPRO Ruby stain analysis of AAVX HPLC vs iodixanol ultracentrifugation purified viruses. Most preps show clear distinct VP1-VP3 bands, with few non-specific bands present, indicating comparable purity to IDX purified virus. FIG. 4B depicts in vitro infectivity of Anc80 and AAV2 on HEK 293 cells. FIG. 4C provides representative images and quantification of empty capsid ratio via negative-stained transmission electron microscopy of AAVX purified self-complementary AAV9 and self-complementary PHP.eB preps. Left—two representative images each for scAAV9 and scPHP.eB; right—quantification of all preps analysed. Approximately N=200 particles were counted for each prep from two separate images by two blinded researchers. Also see Fig. S5 and Fig. S6 for full images of SYPRO Ruby gels and SEM micrographs respectively.

FIGS. 5A-5D depicts In vivo bioactivity of AAVX-HPLC and iodixanol ultracentrifugation purified AAV. FIG. 5A shows quantification of viral DNA and GFP RNA and protein levels in the liver, brain and quadriceps of mice injected with a total of 10¹¹ vg/mouse of scAAV9-Cbh-GFP. DNA and RNA were quantified using qPCR and qRT-PCR respectively, and protein using Simple Wes. Statistical significance was assessed using two-way ANOVA with Šídák's post-hoc tests. Statistically not significant differences are not shown on the figure, except for AAVX vs iodixanol groups. FIG. 5B show imaging analysis of livers sectioned, stained for tomato lectin and DAPI, and imaged for native GFP fluorescence, tomato lectin and DAPI. FIG. 5C is a comparison of native GFP averaged from 400-700 cells per animal. FIG. 5D show percent of cells that are GFP positive, counted as cells with a higher mean fluorescence intensity than the highest mean fluorescence intensity observed in the vehicle group. Statistical significance was assessed using one-way ANOVA with Tukey's post-hoc test for (C) and two-tailed t-test for (D).Ns=p>0.05; *=p<0.05, **=p<0.01, ***=p<0.001.

FIG. 6 depicts HPLC chromatogram of AAV2 purification from one hyperflask. The chromatogram shows a tight elution peak with a corresponding drop in the pH, as the elution buffer is applied to the column. The Inset depicts a chromatogram of the whole purification with the major UV plateau corresponding to the sample application stage.

FIGS. 7A-7E depicts AAV purification at small scale over multiple cycles with Pluronic F-68 added to 0.1% vol/vol to all buffers. FIG. 7A depicts a schematic of the experiment. FIG. 7B depicts qPCR quantification of AAV vector genomes in different fractions, along preps 1-6. FIG. 7C depicts a comparison of total AAV vector genomes after elution and filtration+buffer exchange with or without Pluronic F-68. Addition of Pluronic F-68 does not increase yields at the elution step, but shows a trend towards increased yields at the filtration+buffer exchange step. FIGS. 7D-7E depicts NGS quantification of unique barcode count from the elution fractions of the 2nd prep (D) and 5th prep (E). Majority of barcodes come from the target prep, indicating low carryover contamination. P-values indicated above bars, determined via two-way ANOVA with Šídák's multiple comparisons test.

FIGS. 8A-8C shows how stringent resin cleaning enables repeated resin re-use at large scale. Input from 1 hyperflask at each step was purified without changing the resin and AAV in input lysate, flow-through and elution tittered using qPCR. The process was repeated for PHP.eB (FIG. 8A) and AAV9 using new batches of resin for each (FIG. 8B). AAV applied at room temperature, at 2 min residence time, 3 mL resin, eluted using pH 2.5 Glycine and resin regenerated using 1 ml/min flow of 0.1M pH1 Phosphoric acid followed by 1 ml/min flow of 6M Guanidine HCl for 15 minutes each. (FIG. 8C) No increase in % of AAV in flow-through was seen throughout 6 cycles. #some eluate lost due to operator error. #—some sample was lost due to handling error

FIG. 9 depicts percent AAV lost at each step of high-efficiency protocol. Largest losses occur at the elution (˜20% of input) and buffer exchange (˜10% of input) steps. Data from FIG. 4 with AAV9 and PHP.eB combined, with N=6 for each.

FIG. 10 depicts uncropped gels of silver stain analysis of AAV capsids from FIG. 4A.

FIG. 11 depicts full negative stain SEM images of scPHP.eB and scAAV9 preps described in FIG. 4C. Each image represents a separate prep. In quantification, a minimum of two images were taken and quantified for each prep.

FIG. 12 depicts images used for GFP fluorescence intensity analysis shown on FIG. 6B. Every image corresponds to a different animal within the groups denoted on the left.

FIG. 13 depicts individual cell GFP mean fluorescence intensities of animals injected with AAVX HPLC or iodixanol ultracentrifugation purified AAV. Every column represents one animal and 3 images were used per animal, resulting in a total of 400-700 cells analysed per animal. Horizontal dotted line represents the mean fluorescence intensity above which cells were counted as GFP positive.

FIG. 14 depicts a multiple sequence alignment of AAVs used to construct the phylogenetic tree depicted in FIG. 1B.

FIGS. 15-17 have been intentionally skipped.

FIG. 18 provides sequence IDs of AAVs depicted in FIG. 1B. Rows and Columns are different capsids and the cells represent the % identify between the two proteins.

FIG. 19 depicts Newick formatted phylogeny of the phylogenetic tree depicted on FIG. 1B.

FIGS. 20-32 depict a complete specification of run parameters for an HPLC system to be used in the instant methods.

FIG. 33 depicts a reasonably high elution peak, signifying efficient production and purification. Left image depicts a chromatogram of the full run, whereas the right image depicts a magnified elution UV peak.

FIG. 34 depicts a low elution peak, signifying inefficient production and/or purification. The left image depicts a chromatogram of the full run, whereas the right image depicts a magnified elution UV peak.

### DETAILED DESCRIPTION OF THE INVENTION

An increased demand for AAV production has led to the need to develop more versatile and scalable production methods. Chromatography has been considered a possible solution but its application to this problem has been hampered by the lack of resins or processes that can purify multiple AAV serotypes without individual optimization⁹⁻¹².

The main advantages of chromatographic purification are its scalability to larger volumes and reduced requirement for hands-on time, which considerably eases AAV manufacturing. Chromatographic resins can be scaled to high volumes, which enables input of possibly unconcentrated large volumes of lysates. The process can also be automated and precisely controlled, monitored and quantified, which eases troubleshooting and provides rich data about the quality of the run. For these reasons, chromatography based methods have become the main workhorse of industrial AAV production, as well as industrial production of other biologics and small molecules⁸. It was discovered that AAVX affinity chromatography allows for purification of multiple AAV serotypes at multiple scales, is efficient, and results in virus of comparable purity and bioactivity to ultracentrifugation purified virus.

Disclosed herein, are methods of high efficiency purification of adeno-associated virus (AAV) particles comprising the steps of a) providing a cell lysate that comprises one or more AAV particles; b) contacting the cell lysate comprising the one or more AAV particles with a volume of chromatography resin medium comprising at least one ligand possessing a pan-AAV affinity, wherein said contacting induces binding of the one or more AAV particles to the chromatography resin medium and wherein the contacting is performed at a temperature of between approximately 20° C. to approximately 28° C. and/or both the cell lysate and the chromatography resin medium are allowed to come to a temperature between approximately 20° C. to approximately 28° C. previous to said contacting; and c) eluting the bound one or more AAV particles from the chromatography resin medium using a volume of an acidic elution buffer solution to provide one or more eluted volume fractions containing the one or more AAV particles; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, and wherein the method does not require modifications contingent on the AAV serotype being purified to obtain said efficiency.

In order to enhance the efficiency and bioactivity of the purified AAVs, one or more of the purification steps are performed at room temperature. For purposes of this invention, room temperature is defined as a temperature between approximately 20° C. and approximately 28° C. In some embodiments the contacting and elution steps are not carried out below a temperature of 20° C., 21° C., 22° C., 23° C., 24° C. or 25° C. or are not carried out above a temperature of 24° C., 25° C., 26° C., 27° C., 28° C., 29° C. or 30° C. In a preferred embodiment, the contacting or elution steps are performed at a temperature of approximately 21° C. to approximately 26° C., approximately 22° C. to approximately 25° C., or approximately 23° C. to approximately 24° C. In one preferred embodiment, either one or both of the contacting and elution steps are performed at a temperature of approximately 21° C., 23° C., or approximately 24° C.

In other embodiments, binding efficiency of the AAV particles within the cell lysate to the chromatography resin medium may be enhanced by allowing one or both of the cell lysate and chromatography resin medium to come to a temperature between approximately 21° C. to approximately 26° C., approximately 22° C. to approximately 25° C., or approximately 23° C. to approximately 24° C. In one preferred embodiment, either one or both of the cell lysate and chromatography resin medium are allowed to come to a temperature of approximately 21° C., 23° C., or approximately 24° C. In a particularly preferred embodiment, one or more steps of the method are performed at room temperature as defined herein and both the cell lysate and chromatography resin medium is allowed to attain room temperature prior to one or more steps of the method.

Also disclosed herein are methods of high efficiency purification of adeno-associated virus (AAV) particles comprising the steps of a) providing a cell lysate that comprises one or more AAV particles; b) contacting the cell lysate comprising the one or more AAV particles with a volume of chromatography resin medium comprising at least one ligand possessing a pan-AAV affinity, wherein said contacting induces binding of the one or more AAV particles to the chromatography resin medium; and c) eluting the bound one or more AAV particles from the chromatography resin medium using a volume of an acidic elution buffer solution to provide one or more eluted volume fractions containing the one or more AAV particles; whereby purified AAV particles are obtained with an overall efficiency of at least 65% regardless of the serotype of the AAV purified, wherein at least one of the method steps is carried out in the presence of a nonionic surfactant, wherein the purity, yield and bioactivity of the AAV particles purified thereby are comparable to the purity, yield and bioactivity of iodixanol purified AAV particles, wherein the method does not require modifications contingent on the AAV serotype being purified to obtain said efficiency.

In some embodiments, two or more steps, three or more steps, four or more steps, five or more steps or substantially all of the steps of the method may be performed in the presence of a non-ionic surfactant. In some embodiments, any surface which will come into contact with the AAV particles as described herein may be treated with a non-ionic surfactant to reduce the likelihood that the AAV particles adhere thereto. Such treatment may be performed by first diluting the non-ionic surfactant within a suitable carrier, solvent or diluent and spraying onto, submerging, wiping or soaking the substrate with the diluted non-ionic surfactant for a predetermined amount of time. In some embodiments, the treatment is a function of performing the steps of the method with buffer or other solutions which already contain the non-ionic surfactant. In some embodiments, the concentration of the non-ionic surfactant within the diluted solution is between 0.001% to 20%, between 0.005% to 10%, between 0.01% to 5% or between 0.5% and 3%. In preferred embodiments, the concentration of the non-ionic surfactant in a lysing solution is 0.001%, the concentration of the non-ionic surfactant in an elution buffer is 0.01%, the concentration of the non-ionic surfactant in a neutralization buffer is 0.1% and the concentration of the non-ionic surfactant in a final formulation buffer is 0.001%.

In some embodiments, the non-ionic surfactant is a tri-block copolymer. In still further embodiments, the non-ionic surfactant is a tri-block poly(ethylene oxide) (PEO)-poly(propylene oxide) (PPO)-poly(ethylene oxide) (PEO) copolymer. In some embodiments, the non-ionic surfactant is a commercially available pluronic polymer which shares compatibility with one or more other reagents disclosed throughout the disclosure. In a preferred embodiment, the non-ionic surfactant is PLURONIC F-68.

Also disclosed herein are methods of high efficiency purification of adeno-associated virus (AAV) particles comprising the steps of a) providing a cell lysate that comprises one or more AAV particles; b) contacting the cell lysate comprising the one or more AAV particles with a volume of chromatography resin medium comprising at least one ligand possessing a pan-AAV affinity, wherein said contacting induces binding of the one or more AAV particles to the chromatography resin medium; c) eluting the bound one or more AAV particles from the chromatography resin medium using a volume of an acidic elution buffer solution to provide one or more eluted volume fractions containing the one or more AAV particles; and d) regenerating the chromatography resin medium comprising the at least one ligand possessing a pan-AAV affinity by contacting the medium with at least one acidic component, wherein contact with the at least one acidic component is carried out for a predetermined amount of time, whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, and wherein the method does not require modifications contingent on the AAV serotype being purified to obtain said efficiency.

In some embodiments, the at least one acidic component utilized in the step of regeneration of the chromatography resin medium comprises any suitable binary acid, oxyacid or carboxylic acid which are commercially available and which are suitable for contact with an antibody fragment without causing denaturation, unfolding or loss of its AAV capsid binding activity. In some embodiments the acid is a weak acid and comprises one or more of formic acid, phosphoric acid and/or guanidine HCL. In some embodiments, the at least one acidic component comprises two acidic components which are each contacted with the chromatography resin concurrently or sequentially, and wherein the contact with each of the two acidic components is carried out for predetermined amounts of time. In some embodiments, the predetermined amount of time is 30 seconds to 24 hours, 1 minute to 12 hours, 5 minutes to 4 hours, 10 minutes to 1 hour, or 15 minutes to 30 minutes.

In some embodiments, each predetermined amount of time is at least 15 minutes. In some embodiments, the regeneration step is carried out by contacting the chromatography resin medium with 0.1M phosphoric acid for at least 15 minutes, followed or preceded by contacting the chromatography resin medium with 6M guanidine for at least 15 minutes. In some embodiments, the step of regeneration of the chromatography resin medium is performed a second time, a third time, a fourth time, a fifth time, a sixth time, a seventh time, an eighth time, a ninth time or ten or more times.

It will be appreciated that one of the important objectives of the invention is to provide a purification platform and protocol to enable the efficient large scale purification of AAV particles. Thus, in other embodiments, and especially where it is not practical to scale up the volume of chromatography resin medium used in the method, the method of the invention comprises repetition of steps a) through c) in order to facilitate purification of large quantities of AAV particles. Such large scale purification may include purification of different batches of cell lysates, each batch of cell lysate containing a distinct AAV particle serotype. Alternatively, each batch of cell lysate utilized in a large scale purification process made containing cell lysate batches that comprise the same AAV particle serotype. In some embodiments, the step of regeneration of the chromatography resin medium is performed before a repetition of steps a) through c). In some embodiments, the completion or repetition of the step of regeneration of the chromatography resin medium results in one or both of i) sustaining high efficiency purification during subsequent repetitions of steps a) through c) and ii) eliminating carry over contamination between repetitions of steps a) through c).

As disclosed herein, one important and inventive aspect of the herein disclosed method is the ability to achieve highly efficient purification of widely divergent AAV serotypes, without further optimizations contingent on the serotype to be purified. In some embodiments, the AAV serotypes that can be efficiently purified through use of this method are not particularly limited and include both natural and synthetic AAV serotypes. In one embodiment, the one or more AAV serotypes selected for purification with the method are selected from the group consisting of AAV1, AAV2, AAV2-7m8, AAV-HSPG, AAV3, AAV4, AAV5, AAV6, AAV6.2, AAV7, AAV8, AAV9, Rh10, Rh61, Rh8, Rh32.33, VR-865, PHP.B and Anc80.

In some embodiments, the volume of acidic elution buffer solution comprises glycine, phosphoric acid or citric acid. In some embodiments, the volume of acidic elution buffer solution comprises glycine. In some embodiments, the glycine is present in a concentration of about 0.1M to about 2M, about 0.2M to about 1M or is present in a concentration of about 0.2M. In some embodiments, the pH of the acidic elution buffer solution is approximately 3.0 or less. In some embodiments, the pH of the acidic elution buffer solution is between about 2 to about 2.5. In some further embodiments, the acidic elution buffer solution comprises both glycine and a non-ionic surfactant. In still other embodiments, the nonionic surfactant is PLURONIC F-68. In a particularly preferred embodiment, the acidic elution buffer solution comprises 0.2M glycine, 0.01% PLURONIC F-68 and has a pH of between about 2 and about 2.5.

In some embodiments, the chromatography resin medium comprising a ligand possessing a pan-AAV affinity comprises a cross-linked poly(styrene-divinylbenzene) bead coated with a cross-linked polyhydroxylated polymer. In some embodiments, the chromatography resin medium is linked to a ligand which comprises a camelid heavy-chain-only single domain antibody fragment. In some embodiments, the camelid antibody was raised against a conserved region of an AAV capsid. In some embodiments, the chromatography resin medium comprises one or more beads with a diameter of about 30-70 μm, about 40-60 μm or approximately 50 μm. In a particularly preferred embodiment, the chromatography resin medium is POROS CAPTURESELECT AAVX resin available from Thermo Fisher in a prepacked or free form.

In some embodiments, the one or more purified AAV particles comprise capsids which are filled with vector DNA. In some embodiments, the one or more purified AAV particles comprise capsids which are empty.

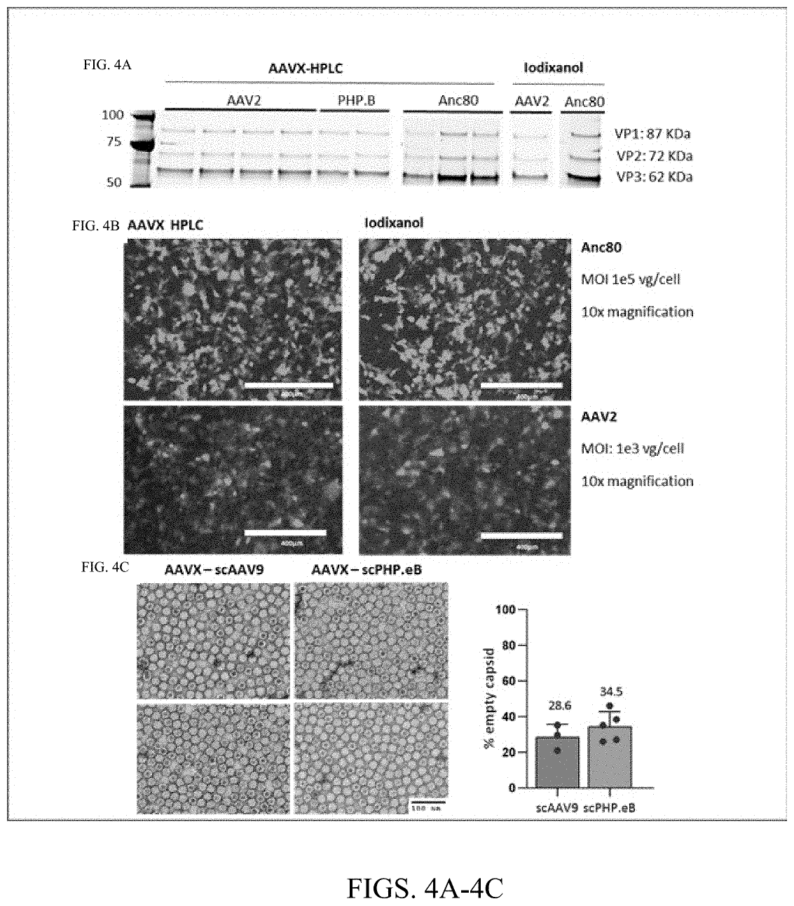

Several reports have described empty capsid copurification to various degrees with affinity and other types of chromatography^{11,12,28} It was discovered that the percent of empty capsids in AAV9 and PHP.eB preps purified using AAVX affinity chromatography to be approximately 30% (FIG. 4C). Empty capsid percentage was estimated using negative stain transmission electron microscopy. Electron microscopy has the advantage of producing a clear visual of the AAV particle populations present, but can suffer from potential image noise, staining artefacts or experimenter subjectivity at quantification³⁸. Nevertheless, when carried out rigorously, electron microscopy based estimation of empty capsids can closely match that of analytical ultracentrifugation³⁹.

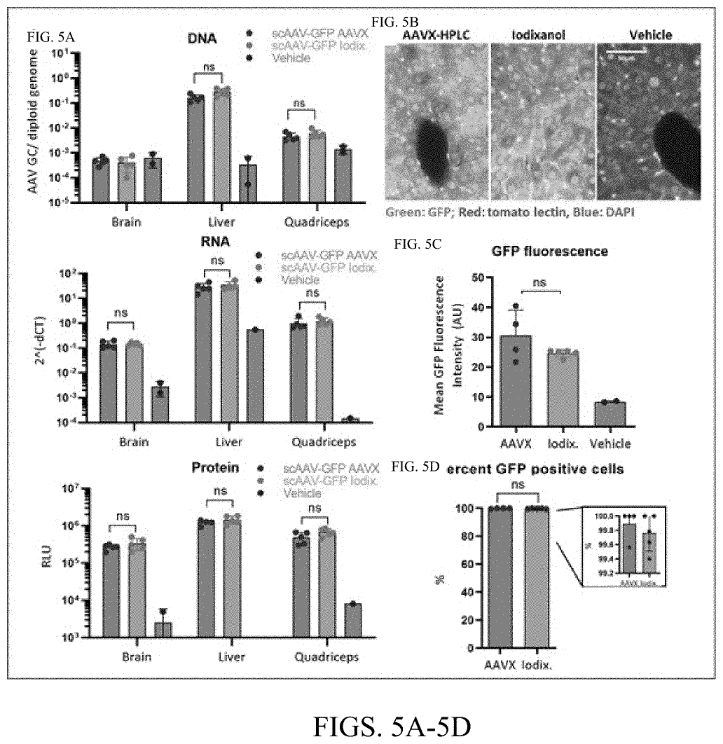

In some embodiments, where the presence of some level of empty capsids is tolerated, no additional step is performed to separate empty capsids from capsids which contain vector DNA. In some embodiments, the presence of empty capsids may not substantially change the outcome of gene transfer utilizing the purified AAV particles disclosed herein. Indeed, the data indicate an equivalent in vivo gene transfer efficacy across multiple organs, inclusion of empty capsids notwithstanding (FIG. 5A).

In some embodiments, however, maximal reduction of empty capsid content is desirable or required. Thus, in some embodiments, various upstream or downstream steps that reduce production of empty capsids or enrich for full capsids can be added. These include optimization of plasmid transfection ratios²⁹; use of vector plasmids that are full length or with minimal ITR deletion²⁹; use of novel engineered ITRs; use of a transfection plasmid containing both the AAV cap and transgene in cis²⁹, or other methods which have been reported to reduce the fraction of empty capsids in the input lysate. In some embodiments, multiple different downstream steps to enrich for full capsids include utilizing, size exclusion, anion exchange, or other chromatographic methods^{9,10,12,30}-37 which are included within the scope of this invention. In some embodiments, these can be added in series as additional steps to the process after the AAVX affinity binding step.

In some embodiments, the method further comprises a step of neutralizing the one or more eluted fractions containing the one or more AAV particles. In some embodiments, the step of neutralizing comprises addition of tris(hydroxymethyl)aminomethane hydrochloride (TRIS-HCL) to the one or more elution fractions. In some embodiments, the TRIS-HCL is added to one or more eluted fractions in combination with a non-ionic surfactant in a neutralization buffer. In some embodiments, the neutralization buffer has a pH of about 7 to about 9. In some preferred embodiments, the neutralization buffer has a pH of about 8. In a particularly preferred embodiment, the neutralization step comprises addition of a neutralization buffer comprising 1M TRIS-HCL and 0.1% PLURONIC F68 to the one or more eluted fractions containing the one or more AAV particles, wherein the neutralization buffer has a pH of about 8.

In some embodiments, the method further comprises one or more washing steps prior to, after, or both prior to and after the elution step. In some embodiments, the one or more washing steps are carried out with a washing buffer solution comprising one or more components selected from the group consisting of tris-buffered saline (TB S), ethanol, guanidine HCL, phosphoric acid, glycine, Tris, NaOH, water, a non-ionic surfactant, and NaCl. In some embodiments, the washing step is performed after the contacting step, but before the eluting step. In some embodiments, the wash buffer solution comprises ethanol. In some embodiments, the washing buffer comprises ethanol and TBS.

In some embodiments, the chromatography resin medium linked to at least one ligand which possess pan-AAV affinity is packed into a column to provide a high performance liquid chromatography column. In some embodiments, the chromatography resin medium linked to at least one ligand which possess pan-AAV affinity is purchased pre-packed into a column. In some embodiments, the volume of chromatography resin medium is at least 0.1 mL, at least 0.5 mL, at least 1 mL, at least 2 mL, at least 3 mL, at least 4 mL, at least 5 mL, or at least 10 mL. In some embodiments, the volume of chromatography resin medium is no more than 10 L, no more than 5 L, no more than 1 L, no more than 100 mL, no more than 10 mL, no more than 5 mL, no more than 2 mL, or no more than 1 mL. In a preferred embodiment, the volume of chromatography resin medium is approximately 1 mL.

In some embodiments, a flow rate of one or both of the acidic elution buffer solution and the wash buffer solution through the chromatography resin medium during the elution or washing steps comprises a flow rate of no more than 5 mL/min, no more than 4 mL/min, no more than 3 mL/min, no more than 2 mL/min, no more than 1 mL/min, no more than 0.5 ml/min or no more than 0.1 ml/min. In some embodiments, a flow rate of one or both of the acidic elution buffer solution and the wash buffer solution through the chromatography resin medium during the elution or washing steps comprises a flow rate per minute of approximately an equal volume or less of the acidic elution buffer solution or wash buffer solution per volume of chromatography resin medium. In some embodiments, a flow rate of one or both of the acidic elution buffer solution and the wash buffer solution is 0.5 mL/min to 1 mL/min. In some embodiments, the volume of the chromatography resin medium is approximately 1 mL. In some embodiments, the flow rate of one or both of the acidic elution buffer solution and wash buffer solution is approximately 1 mL/min. In some embodiments, the volume of the chromatography resin medium is approximately 1 mL and the flow rate of one or both of the acidic elution buffer solution and wash buffer solution is 1 mL/min. In some embodiments, the volume of the chromatography resin medium is approximately 1 mL and the flow rate of one or both of the acidic elution buffer solution and wash buffer solution is 0.5 mL/min. In some embodiments, the volume of the chromatography resin medium is approximately 1 mL and the flow rate of one or both of the acidic elution buffer solution and wash buffer solution is 0.25 mL/min.

In other embodiments, a direction of flow of the elution acidic buffer solution in an elution step is opposite to a direction of flow of the cell lysate through the chromatography resin medium in the contacting step, whereby an increased quantity of AAV particles are eluted from the chromatography resin medium compared to when the directions of flow in the elution and providing steps are the same. While not wishing to be bound by theory, it is believed that when the cell lysate is contacted in a first flow direction with chromatography resin medium that has been packed into a column, AAV particles in the cell lysate will bind quickly to the chromatography resin medium very close to the point of introduction of the cell lysate. Thus, it follows that if an elution buffer is introduced in the introduction point with the same direction of flow, AAV particles may rebind to the column before they can be eluted. On the contrary, when the elution buffer is introduced at a point distal to the cell lysate introduction point and an opposite direction of flow of the elution buffer is utilized, AAV particles released from the chromatography resin will encounter far less volume of chromatography resin medium, resulting in more AAV particle eluting during this step.

In some embodiments, the method further comprises a step of capturing the at least one or more elution volume fractions. In some embodiments, the step of capturing the at least one or more elution volume fractions includes collecting all of the elution volume fraction in a single container. In some embodiments, the step of capturing the at least one or more elution volume fractions includes collecting one or more of the elution volume fractions in separate container. In some embodiments, the elution volume fractions are subjected to qPCR to determine the quantity of vector DNA present in the elution volume fraction.

In some embodiments, the method further comprises a step of sterilizing the at least one or more elution volume fractions containing the purified one or more AAV vector particles. In some embodiments, the method comprises a further step of neutralizing the one or more elution volume fractions after collection. In some embodiments, the step of neutralizing comprises addition of tris(hydroxymethyl)aminomethane hydrochloride (TRIS-HCL) to the one or more elution fractions. In some embodiments, the TRIS-HCL is added to one or more eluted fractions in combination with a non-ionic surfactant in a neutralization buffer. In some embodiments, the neutralization buffer has a pH of about 7 to about 9. In some preferred embodiments, the neutralization buffer has a pH of about 8. In a particularly preferred embodiment, the neutralization step comprises addition of a neutralization buffer comprising 1M TRIS-HCL and 0.1% PLURONIC F68 to the one or more eluted fractions containing the one or more AAV particles, wherein the neutralization buffer has a pH of about 8. In some embodiments, the neutralized one or more elution volume fractions are sterilized via a polyethersulfone syringe or cap filter. In some embodiments, the polyethersulfone syringe or cap filter is a 0.22 μm filter.

In some embodiments, the method further comprises the step of submitting the at least one or more elution volume fractions to a buffer exchange. In some embodiments, the one or more elution volume fractions may be neutralized elution volume fraction. In some embodiments, the buffer exchange occurs in an AMICON Stirred Cell concentrator or an AMICON Ultra-15 filter concentrator. In some embodiments the elution volume fractions are submitted to buffer exchange and concentrated with a molecular weight cut-off of 50 kDa or 100 kDA. In some embodiments the buffer exchange and/or concentration occurs under influence of an inert gas. In some embodiments, the inert gas is nitrogen.

Also disclosed herein are methods of high efficiency purification of adeno-associated virus (AAV) particles comprising the steps of a) transfecting at least one cell with plasmid DNA comprising an AAV vector genome, one or more AAV capsid genes and one or more transacting helper genes; b) providing a cell lysate that comprises one or more AAV particles by lysing the transfected at least one cell in situ; c) contacting the cell lysate comprising the one or more AAV particles with a volume of chromatography resin medium comprising at least one ligand possessing a pan-AAV affinity, wherein said contacting induces binding of the one or more AAV particles to the chromatography resin medium; and d) eluting the bound one or more AAV particles from the chromatography resin medium using a volume of an acidic elution buffer solution to provide one or more eluted volume fractions containing the one or more AAV particles; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, wherein the method does not require modifications contingent on the AAV serotype being purified to obtain said efficiency and wherein at least one of the following is true: i) at least one of the method steps is carried out in the presence of a non ionic surfactant, ii) the contacting is performed at a temperature of between approximately 20° C. to approximately 28° C., iii) both the cell lysate and the chromatography resin medium are allowed to come to a temperature between approximately 20° C. to approximately 28° C. previous to said contacting, or iv) the method further comprises a step of regenerating the chromatography resin medium comprising the at least one ligand possessing a pan-AAV affinity by contacting the medium with at least one acidic component for a predetermined amount of time.

In some embodiments, prior to transfection, cells are initially seeded and subsequently expanded on a suitable substrate. Such a substrate is not particularly limited and may include any substrate which comprises a planar or non-planar horizontal surface and means for retaining a liquid and cells placed thereon. In some embodiments, the substrate is selected from one or more dishes having a desired capacity. In some embodiments, the dish is a dish with a 15 mL capacity. In some embodiments, the substrate is a hyperflask, a shaker flask or a CellSTACK cell culture chamber. In a particularly preferred embodiment, the substrate is a hyperflask. In some embodiments, the seeding and expansion of cells may be initiated on a first substrate and continued to desired confluency on a second substrate. In a preferred embodiment, cells are seeded and expanded on a dish until 80% confluency and are subsequently transferred to a hyperflask until 80% confluency is achieved. In some embodiments, a confluency of greater than 90% is avoided.

In a preferred embodiment, the substrate contains a cell growth medium. In some embodiments, the cell growth medium is not particularly limited and encompasses cell growth medium known to those skilled in the art. In a preferred embodiment, the cell growth medium comprises Dulbecco's Modified Eagle Medium. In some embodiments, the cell growth medium comprises DMEM, 10% fetal bovine serum (FBS) and 1% Pen/Strep. In some embodiments the cell growth medium has been sterilized, such as by filter sterilization. In some embodiments, the cell growth medium is warmed to approximately 37° C. prior to use. In some embodiments, the seeding and expansion of cells is carried out at an elevated temperature compared to room temperature. In some embodiments, the seeding and expansion of cells is carried out at approximately 37° C.

In some embodiments, the cells transfected by vector DNA are not particularly limited and include those cells known to a skilled artisan for this purpose, such as those which are highly transfectable. In some embodiments, the cells transfected by vector DNA is one selected from HeLa, HEK293, 293-T, sBHK and Sf9. In a preferred embodiment, the cell transfect by vector DNA is HEK293.

In some embodiments, the step of transfection comprises mixing DMEM with DNA. In some embodiments, the DNA comprises vector DNA, AAV capsid DNA and DNA of a helper plasmid. In some embodiments, the vector DNA, AAV capsid DNA and DNA of a helper plasmid are provided in a weight ratio of 1:1:2. In some embodiments, the helper plasmid is adeno-helper plasmid deltaF6. In some embodiments, the DMEM, vector DNA, AAV capsid DNA and DNA of a helper plasmid are mixed with PEIMax, and DMEM containing 1% PenStrep to provide a mixture for transfecting a cell, which mixture does not contain serum. In some embodiments, the mixture for transfecting a cell does no contain fetal bovine serum. In some embodiments, the transfection step includes mixing the DMEM, vector DNA, AAV capsid DNA and helper plasmid DNA, PEIMax with cells to be transfected, and is subsequently incubated for 3-5 days.

In some embodiments, the step of providing cell lysate comprises mixing a non-ionic detergent with one or more nucleases. In some embodiments, the one or more nucleases comprise Turbonuclease. In some embodiments, the non-ionic detergent comprises TRITON-X-100 and the nuclease comprises Turbonuclease. In some embodiments, the step of providing cell lysate is carried out in the presence of PLURONIC F68 at a temperature which is elevated compared to room temperature. In some embodiments, the step of providing cell lysate by in situ lysing is carried out at a temperature of approximately 37° C.

In order to enhance the efficiency and bioactivity of the purified AAVs, one or more of the providing, contacting and eluting steps are performed at room temperature. For purposes of this invention, room temperature is defined as a temperature between approximately 20° C. and approximately 28° C. In some embodiments the contacting and elution steps are not carried out below a temperature of 20° C., 21° C., 22° C., 23° C., 24° C. or 25° C. or are not carried out above a temperature of 24° C., 25° C., 26° C., 27° C., 28° C., 29° C. or 30° C. In a preferred embodiment, the contacting or elution steps are performed at a temperature of approximately 21° C. to approximately 26° C., approximately 22° C. to approximately 25° C., or approximately 23° C. to approximately 24° C. In one preferred embodiment, either one or both of the contacting and elution steps are performed at a temperature of approximately 21° C., 23° C., or approximately 24° C.

In other embodiments, binding efficiency of the AAV particles within the cell lysate to the chromatography resin medium may be enhanced by allowing one or both of the cell lysate and chromatography resin medium to come to a temperature between approximately 21° C. to approximately 26° C., approximately 22° C. to approximately 25° C., or approximately 23° C. to approximately 24° C. In one preferred embodiment, either one or both of the cell lysate and chromatography resin medium are allowed to come to a temperature of approximately 21° C., 23° C., or approximately 24° C. In a particularly preferred embodiment, one or more steps of the method are performed at room temperature as defined herein and both the cell lysate and chromatography resin medium is allowed to attain room temperature prior to one or more steps of the method.

In some embodiments, two or more steps, three or more steps, four or more steps, five or more steps or substantially all of the steps of the method may be performed in the presence of a non-ionic surfactant. In some embodiments, any surface which will come into contact with the AAV particles as described herein may be treated with a non-ionic surfactant to reduce the likelihood that the AAV particles adhere thereto. Such treatment may be performed by first diluting the non-ionic surfactant within a suitable carrier, solvent or diluent and spraying onto, submerging, wiping or soaking the substrate with the diluted non-ionic surfactant for a predetermined amount of time. In some embodiments, the treatment is a function of performing the steps of the method with buffer or other solutions which already contain the non-ionic surfactant. In some embodiments, the concentration of the non-ionic surfactant within the diluted solution is between 0.001% to 20%, between 0.005% to 10%, between 0.01% to 5% or between 0.5% and 3%. In preferred embodiments, the concentration of the non-ionic surfactant in a lysing solution is 0.001%, the concentration of the non-ionic surfactant in an elution buffer is 0.01%, the concentration of the non-ionic surfactant in a neutralization buffer is 0.1% and the concentration of the non-ionic surfactant in a final formulation buffer is 0.001%.

In some embodiments, the non-ionic surfactant is a tri-block copolymer. In still further embodiments, the non-ionic surfactant is a tri-block poly(ethylene oxide) (PEO)-poly(propylene oxide) (PPO)-poly(ethylene oxide) (PEO) copolymer. In some embodiments, the non-ionic surfactant is a commercially available pluronic polymer which shares compatibility with one or more other reagents disclosed throughout the disclosure. In a preferred embodiment, the non-ionic surfactant is PLURONIC F-68.

In some embodiments, the at least one acidic component utilized in the step of regeneration of the chromatography resin medium comprises any suitable binary acid, oxyacid or carboxylic acid which are commercially available and which are suitable for contact with an antibody fragment without causing denaturation, unfolding or loss of its AAV capsid binding activity. In some embodiments the acid is a weak acid and comprises one or more of formic acid, phosphoric acid and/or guanidine HCL. In some embodiments, the at least one acidic component comprises two acidic components which are each contacted with the chromatography resin medium concurrently or sequentially, and wherein the contact with each of the two acidic components is carried out for predetermined amounts of time. In some embodiments, the predetermined amount of time is 30 seconds to 24 hours, 1 minute to 12 hours, 5 minutes to 4 hours, 10 minutes to 1 hour, or 15 minutes to 30 minutes.

In some embodiments, each predetermined amount of time is at least 15 minutes. In some embodiments, the regeneration step is carried out by contacting the chromatography resin medium with 0.1M phosphoric acid for at least 15 minutes, followed or preceded by contacting the chromatography resin medium with 6M guanidine for at least 15 minutes. In some embodiments, the step of regeneration of the chromatography resin medium is performed a second time, a third time, a fourth time, a fifth time, a sixth time, a seventh time, an eighth time, a ninth time or ten or more times.

It will be appreciated that one of the important objectives of the invention is to provide a purification platform and protocol to enable the efficient large scale purification of AAV particles. Thus, in other embodiments, and especially where it is not practical to scale up the volume of chromatography resin medium used in the method, the method of the invention comprises repetition of at least steps b) through d) in order to facilitate purification of large quantities of AAV particles. In some embodiments, the method includes repetition of one or more of steps a) through d). Such large scale purification may include purification of different batches of cell lysates, each batch of cell lysate containing a distinct AAV particle serotype. Alternatively, each batch of cell lysate utilized in a large scale purification process can comprise cell lysate containing the same AAV particle serotype. In some embodiments, the step of regeneration of the chromatography resin medium is performed before a repetition of steps b) through d). In some embodiments, the completion or repetition of the step of regeneration of the chromatography resin medium results in one or both of i) sustaining high efficiency purification during subsequent repetitions of steps c) through d) and ii) eliminating carry over contamination between repetitions of steps c) through d).

As disclosed herein, one important and inventive aspect of the herein disclosed method is the ability to achieve highly efficient purification of widely divergent AAV serotypes, without further optimizations contingent on the serotype to be purified. In some embodiments, the AAV serotypes that can be efficiently purified through use of this method are not particularly limited and include both natural and synthetic AAV serotypes. In one embodiment, the one or more AAV serotypes selected for purification with the method are selected from the group consisting of AAV1, AAV2, AAV2-7m8, AAV-HSPG, AAV3, AAV4, AAV5, AAV6, AAV6.2, AAV7, AAV8, AAV9, Rh10, Rh61, Rh8, Rh32.33, VR-865, PHP.B and Anc80.

In some embodiments, the volume of acidic elution buffer solution comprises glycine, phosphoric acid or citric acid. In some embodiments, the volume of acidic elution buffer solution comprises glycine. In some embodiments, the glycine is present in a concentration of about 0.1M to about 2M, about 0.2M to about 1M or is present in a concentration of about 0.2M. In some embodiments, the pH of the acidic elution buffer solution is approximately 3.0 or less. In some embodiments, the pH of the acidic elution buffer solution is between about 2 to about 2.5. In some further embodiments, the acidic elution buffer solution comprises both glycine and a non-ionic surfactant. In still other embodiments, the nonionic surfactant is PLURONIC F-68. In a particularly preferred embodiment, the acidic elution buffer solution comprises 0.2M glycine, 0.01% PLURONIC F-68 and has a pH of between about 2 and about 2.5.

In some embodiments, the chromatography resin medium comprising a ligand possessing a pan-AAV affinity comprises a cross-linked poly(styrene-divinylbenzene) bead coated with a cross-linked polyhydroxylated polymer. In some embodiments, the chromatography resin medium is linked to a ligand which comprises a camelid heavy-chain-only single domain antibody fragment. In some embodiments, the camelid antibody was raised against a conserved region of an AAV capsid. In some embodiments, the chromatography resin medium comprises one or more beads with a diameter of about 30-70 μm, about 40-60 μm or approximately 50 μm. In a particularly preferred embodiment, the chromatography resin medium is POROS CAPTURESELECT AAVX resin available from Thermo Fisher in a prepacked or free form.

In some embodiments, the one or more purified AAV particles comprise capsids which are filled with vector DNA. In some embodiments, the one or more purified AAV particles comprise capsids which are empty.

Several reports have described empty capsid copurification to various degrees with affinity and other types of chromatography^{11,12,28} It was discovered that the percent of empty capsids in AAV9 and PHP.eB preps purified using AAVX affinity chromatography to be approximately 30% (FIG. 4C). Empty capsid percentage was estimated using negative stain transmission electron microscopy. Electron microscopy has the advantage of producing a clear visual of the AAV particle populations present, but can suffer from potential image noise, staining artefacts or experimenter subjectivity at quantification³⁸. Nevertheless, when carried out rigorously, electron microscopy based estimation of empty capsids can closely match that of analytical ultracentrifugation³⁹.

In some embodiments, where the presence of some level of empty capsids is tolerated, no additional step is performed to separate empty capsids from capsids which contain vector DNA. In some embodiments, the presence of empty capsids may not substantially change the outcome of gene transfer utilizing the purified AAV particles disclosed herein. Indeed, the data indicate an equivalent in vivo gene transfer efficacy across multiple organs, inclusion of empty capsids notwithstanding (FIG. 5A).

In some embodiments, however, maximal reduction of empty capsid content is desirable or required. Thus, in some embodiments, various upstream or downstream steps that reduce production of empty capsids or enrich for full capsids can be added. These include optimization of plasmid transfection ratios²⁹; use of vector plasmids that are full length or with minimal ITR deletion²⁹; use of novel engineered ITRs; use of a transfection plasmid containing both the AAV cap and transgene in cis²⁹, or other methods which have been reported to reduce the fraction of empty capsids in the input lysate. In some embodiments, multiple different downstream steps to enrich for full capsids include utilizing, size exclusion, anion exchange, or other chromatographic methods^{9,10,12,30-37}, which are included within the scope of this invention. In some embodiments, these can be added in series as additional steps to the process after the AAVX affinity binding step.

In some embodiments, the method further comprises a step of neutralizing the one or more eluted fractions containing the one or more AAV particles. In some embodiments, the step of neutralizing comprises addition of tris(hydroxymethyl)aminomethane hydrochloride (TRIS-HCL) to the one or more elution fractions. In some embodiments, the TRIS-HCL is added to one or more eluted fractions in combination with a non-ionic surfactant in a neutralization buffer. In some embodiments, the neutralization buffer has a pH of about 7 to about 9. In some preferred embodiments, the neutralization buffer has a pH of about 8. In a particularly preferred embodiment, the neutralization step comprises addition of a neutralization buffer comprising 1M TRIS-HCL and 0.1% PLURONIC F68 to the one or more eluted fractions containing the one or more AAV particles, wherein the neutralization buffer has a pH of about 8.

In some embodiments, the method further comprises one or more washing steps prior to, after, or both prior to and after the elution step. In some embodiments, the one or more washing steps are carried out with a washing buffer solution comprising one or more components selected from the group consisting of tris-buffered saline (TB S), ethanol, guanidine HCL, phosphoric acid, glycine, Tris, NaOH, water, a non-ionic surfactant, and NaCl. In some embodiments, the washing step is performed after the contacting step, but before the eluting step. In some embodiments, the wash buffer solution comprises ethanol. In some embodiments, the washing buffer comprises ethanol and TBS.

In some embodiments, the chromatography resin medium linked to at least one ligand which possess pan-AAV affinity is packed into a column to provide a high performance liquid chromatography column. In some embodiments, the chromatography resin medium linked to at least one ligand which possess pan-AAV affinity is purchased pre-packed into a column. In some embodiments, the volume of chromatography resin medium is at least 0.1 mL, at least 0.5 mL, at least 1 mL, at least 2 mL, at least 3 mL, at least 4 mL, at least 5 mL, or at least 10 mL. In some embodiments, the volume of chromatography resin medium is no more than 10 L, no more than 5 L, no more than 1 L, no more than 100 mL, no more than 10 mL, no more than 5 mL, no more than 2 mL, or no more than 1 mL. In a preferred embodiment, the volume of chromatography resin medium is approximately 1 mL.

In some embodiments, a flow rate of one or both of the acidic elution buffer solution and the wash buffer solution through the chromatography resin medium during the elution or washing steps comprises a flow rate of no more than 5 mL/min, no more than 4 mL/min, no more than 3 mL/min, no more than 2 mL/min, no more than 1 mL/min, no more than 0.5 ml/min or no more than 0.1 ml/min. In some embodiments, a flow rate of one or both of the acidic elution buffer solution and the wash buffer solution through the chromatography resin medium during the elution or washing steps comprises a flow rate per minute of approximately an equal volume or less of the acidic elution buffer solution or wash buffer solution per volume of chromatography resin medium. In some embodiments, a flow rate of one or both of the acidic elution buffer solution and the wash buffer solution is 0.5 mL/min to 1 mL/min. In some embodiments, the volume of the chromatography resin medium is approximately 1 mL. In some embodiments, the flow rate of one or both of the acidic elution buffer solution and wash buffer solution is approximately 1 mL/min. In some embodiments, the volume of the chromatography resin medium is approximately 1 mL and the flow rate of one or both of the acidic elution buffer solution and wash buffer solution is 1 mL/min. In some embodiments, the volume of the chromatography resin medium is approximately 1 mL and the flow rate of one or both of the acidic elution buffer solution and wash buffer solution is 0.5 mL/min. In some embodiments, the volume of the chromatography resin medium is approximately 1 mL and the flow rate of one or both of the acidic elution buffer solution and wash buffer solution is 0.25 mL/min.

In other embodiments, a direction of flow of the elution acidic buffer solution in an elution step is opposite to a direction of flow of the cell lysate through the chromatography resin medium in the contacting step, whereby an increased quantity of AAV particles are eluted from the chromatography resin medium compared to when the directions of flow in the elution and providing steps are the same. While not wishing to be bound by theory, it is believed that when the cell lysate is contacted in a first flow direction with chromatography resin medium that has been packed into a column, AAV particles in the cell lysate will bind quickly to the chromatography resin medium very close to the point of introduction of the cell lysate. Thus, it follows that if an elution buffer is introduced in the introduction point with the same direction of flow, AAV particles may rebind to the column before they can be eluted. On the contrary, when the elution buffer is introduced at a point distal to the cell lysate introduction point and an opposite direction of flow of the elution buffer is utilized, AAV particles released from the chromatography resin will encounter far less volume of chromatography resin medium, resulting in more AAV particle eluting during this step.

In some embodiments, the method further comprises a step of capturing the at least one or more elution volume fractions. In some embodiments, the step of capturing the at least one or more elution volume fractions includes collecting all of the elution volume fraction in a single container. In some embodiments, the step of capturing the at least one or more elution volume fractions includes collecting one or more of the elution volume fractions in separate container. In some embodiments, the elution volume fractions are subjected to qPCR to determine the quantity of vector DNA present in the elution volume fraction.

In some embodiments, the method further comprises a step of sterilizing the at least one or more elution volume fractions containing the purified one or more AAV vector particles. In some embodiments, the method comprises a further step of neutralizing the one or more elution volume fractions after collection. In some embodiments, the step of neutralizing comprises addition of tris(hydroxymethyl)aminomethane hydrochloride (TRIS-HCL) to the one or more elution fractions. In some embodiments, the TRIS-HCL is added to one or more eluted fractions in combination with a non-ionic surfactant in a neutralization buffer. In some embodiments, the neutralization buffer has a pH of about 7 to about 9. In some preferred embodiments, the neutralization buffer has a pH of about 8. In a particularly preferred embodiment, the neutralization step comprises addition of a neutralization buffer comprising 1M TRIS-HCL and 0.1% PLURONIC F68 to the one or more eluted fractions containing the one or more AAV particles, wherein the neutralization buffer has a pH of about 8. In some embodiments, the neutralized one or more elution volume fractions are sterilized via a polyethersulfone syringe or cap filter. In some embodiments, the polyethersulfone syringe or cap filter is a 0.22 μm filter.

In some embodiments, the method further comprises the step of submitting the at least one or more elution volume fractions to a buffer exchange. In some embodiments, the one or more elution volume fractions may be neutralized elution volume fraction. In some embodiments, the buffer exchange occurs in an AMICON Stirred Cell concentrator or an AMICON Ultra-15 filter concentrator. In some embodiments the elution volume fractions are submitted to buffer exchange and concentrated with a molecular weight cut-off of 50 kDa or 100 kDA. In some embodiments the buffer exchange and/or concentration occurs under influence of an inert gas. In some embodiments, the inert gas is nitrogen

Also disclosed herein are particularly preferred methods of high efficiency purification of adeno-associated virus (AAV) particles comprising the steps of: a) seeding HEK293T cells onto a substrate which holds a Dulbecco's modified eagle medium (DMEM) containing 10% fetal bovine serum (FBS) and 1% mixture of penicillin G and streptomycin (penstrep) and allowing expansion of the cells until approximately 80% confluency is obtained; b) transfecting the HEK293T cells by contacting said cells with a composition that comprises DMEM, polyethylenimine, penstrep, and plasmid DNA comprising an AAV vector genome, one or more AAV capsid genes and one or more trans-acting helper genes; c) providing a clarified cell lysate that comprises one or more AAV particles by lysing the transfected HEK293T cells in situ utilizing TRITON-X 100, RNAse A, Turbonuclease and PLURONIC F68, subjecting the lysate to centrifugation at 4,000 g or higher and subsequently filtering a supernatant thus obtained by use of a 0.45 μm cellulose acetate/polyethersulfone membrane filter system; d) contacting the clarified cell lysate comprising the one or more AAV particles with a 1 mL volume of POROS CAPTURESELECT AAVX chromatography resin medium, wherein the contacting duration comprises no less than 1 minute, wherein the contacting is performed at a temperature of between approximately 21° C. to approximately 25° C. and/or both the cell lysate and the chromatography resin medium are allowed to come to a temperature between approximately 21° C. to approximately 25° C. previous to said contacting, and wherein said contacting induces binding of the one or more AAV particles to the chromatography resin medium; e) eluting the bound one or more AAV particles from the chromatography resin medium using a volume of a filtered acidic elution buffer solution having a pH between approximately 2.0 and approximately 2.5, to provide one or more eluted volume fractions containing the one or more AAV particles, wherein the filtered acidic elution buffer solution comprises 0.2M glycine and 0.01 v/v % PLURONIC F68 and wherein a flow direction of the acidic elution buffer solution through the chromatography resin medium is opposite a flow direction of the clarified cell lysate through the chromatography resin during the contacting step; f) optionally sterilizing the at least one or more elution volume fractions containing the purified one or more AAV particles utilizing a 0.2 μm polyethersulfone syringe filter; g) subjecting the one or more elution volume fractions containing the purified one or more AAV particles to buffer exchange using either AMICON UTRACEL 15 or AMICON Stirred Cell concentrators, wherein if a 50 or 100 kDA AMICON ULTRA 15 Centrifugal Filter device is used for buffer exchange, less than 1×10¹³ vg of AAV are subjected to buffer exchange therein to reduce sedimentation and loss; and h) regenerating the chromatography resin medium by contacting the chromatography resin medium with 0.1M phosphoric acid for at least 15 minutes, followed or preceded by contacting the chromatography resin medium with 6M guanidine for at least 15 minutes; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, wherein any plastic surface which comes into contact with the AAV particles during the method is first coated with a composition comprising PLURONIC F68, and wherein the method does not require modifications contingent on the AAV serotype being purified to obtain said efficiency.

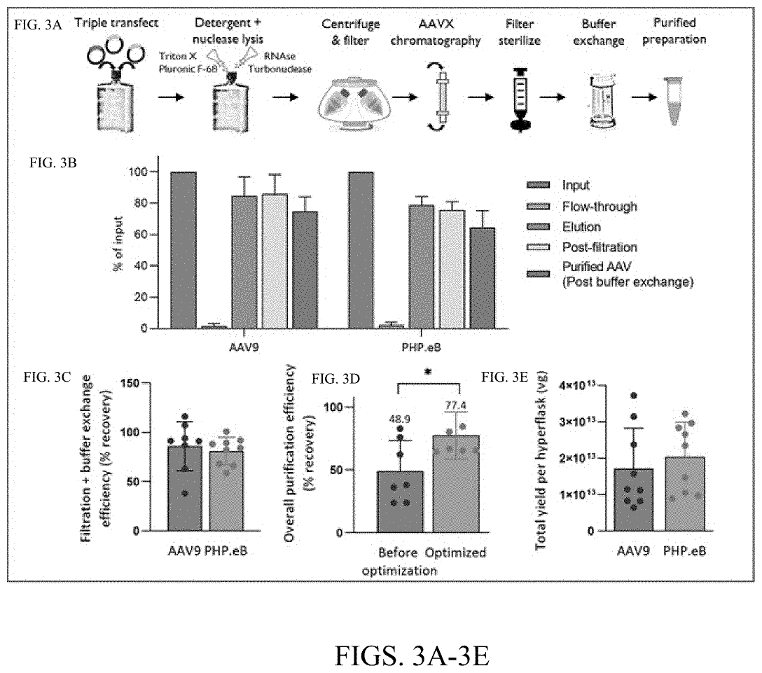

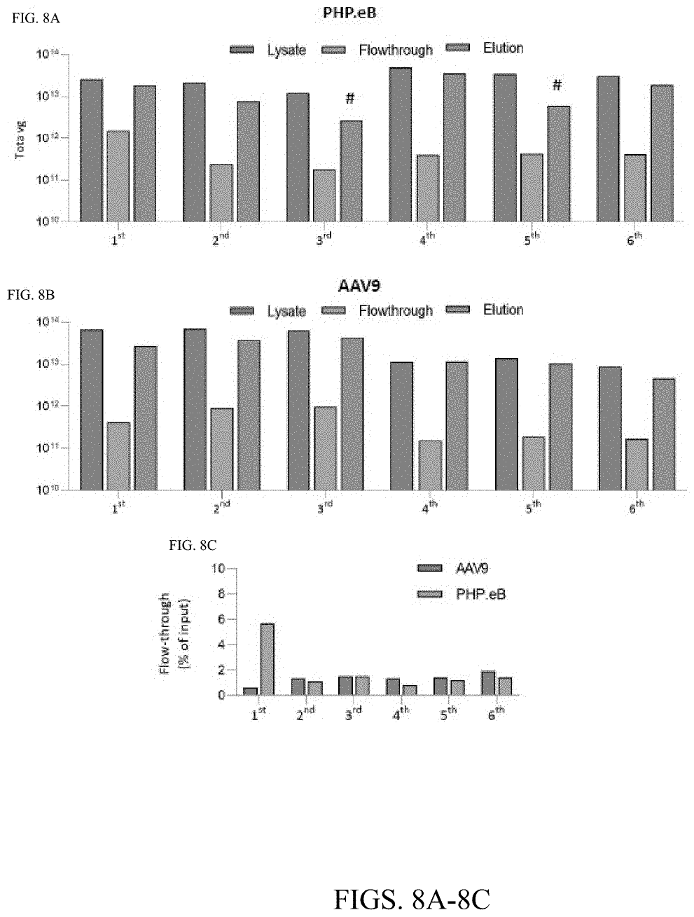

Using AAVX, an optimized and integrated purification process for preps of up to at least 10¹⁴ vector genomes was developed. The main bottlenecks to efficiency include efficient lysis and loss or sedimentation of AAV at the buffer exchange/formulation steps. In some embodiments, in situ lysis using detergents and nucleases as well as buffer exchange using AMICON Stirred Cells were used in order to mitigate these bottlenecks. These and other modifications increased process-wide yields from clarified lysate to purified preparation to an average of approximately 75%, while allowing resin re-use without loss of efficiency for at least 6 purification cycles (FIG. 3C; FIG. 8).

The invention is further described by way of the following Examples.

### EXAMPLES

### AAVX Binds Several AAV Serotypes

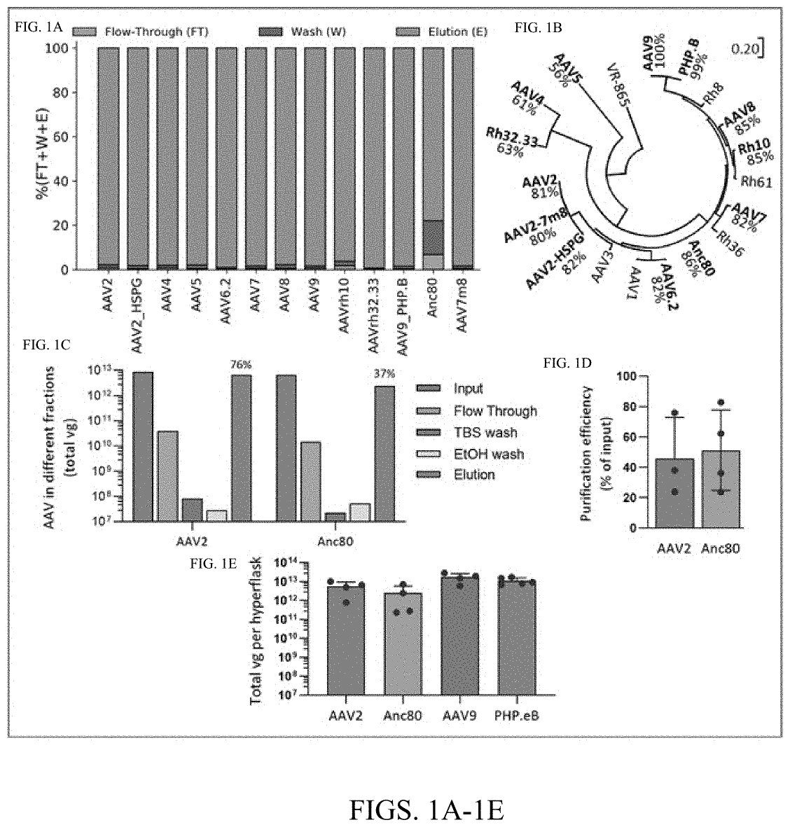

On a small scale, a panel of AAV serotypes were tested to determine whether and which serotypes POROS CaptureSelect AAVX (subsequently denoted as AAVX) can bind. To this end, AAV serotypes AAV2, AAV2_HSPG, AAV4, AAV5, AAV6.2, AAV7, AAV8, AAV9, rh10, rh32.33, PHP.B, Anc80 and AAV7m8^{1,14-23} were produced at a small scale. Crude lysates were incubated with the AAVX resin in a static binding assay (FIG. 1A). The results of these binding assays demonstrate that POROS AAVX can bind all of the tested serotypes with relatively high efficiency. Recovery was >95% for all serotypes except Anc80, which showed around 80% recovery. Thus, AAVX appears versatile in supporting chromatographic binding of multiple different serotypes.

### AAVX Affinity Chromatography can be Used to Purify AAV

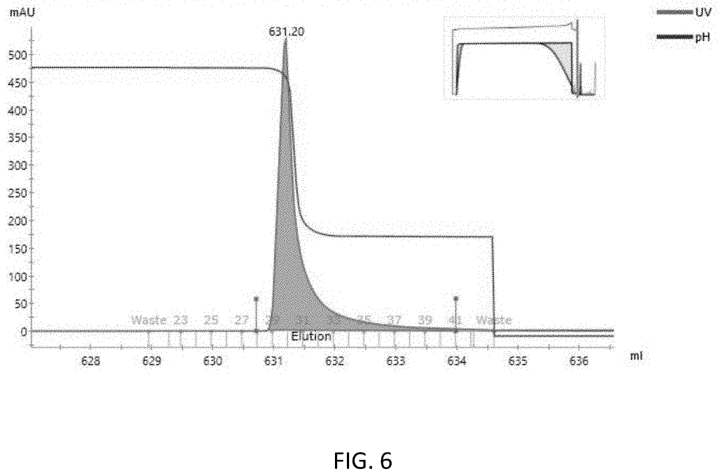

Next, we aimed to determine whether AAVs could be purified with the AAVX resin via HPLC at the larger, hyperflask scale (cell growth surface area of 1720 cm²) from a volume of 560 mL, AAV2 and Anc80 preps based on a single hyperflask were purified on AAVX using HPLC. In short, the production and purification process consisted of triple transfection of adherent HEK293 cells, harvest and high salt lysis 3 days post-transfection, clarification of lysate by centrifugation and filtration, AAVX affinity chromatography at room temperature, 0.22 μm syringe filter sterilization followed by a final buffer exchange and concentration step using 50 kDa molecular weight cut-off filtration (Amicon Ultracel 15). Recovery in each of the different chromatography fractions was quantified by qPCR for DNAse-resistant vector genomes (FIG. 1B, FIG. 6). Results from these experiments indicated that the majority of input virus is found in the elution fraction, with only a minor fraction of virus lost in the flow-through or TBS and ethanol washes. Repeated preps indicated that combined average purification efficiency for both AAV2 and Anc80 without serotype-specific optimization is around 50% (FIG. 1C). The average yield of AAV2 and Anc80 from this initial process was 10¹³ vector genomes (vg) of AAV per hyperflask, which was maintained for the more sequence divergent serotypes AAV9 and PHP.eB (FIG. 1D).

AAVX can be Regenerated for Re-Use without Loss of Efficiency or Carryover Contamination

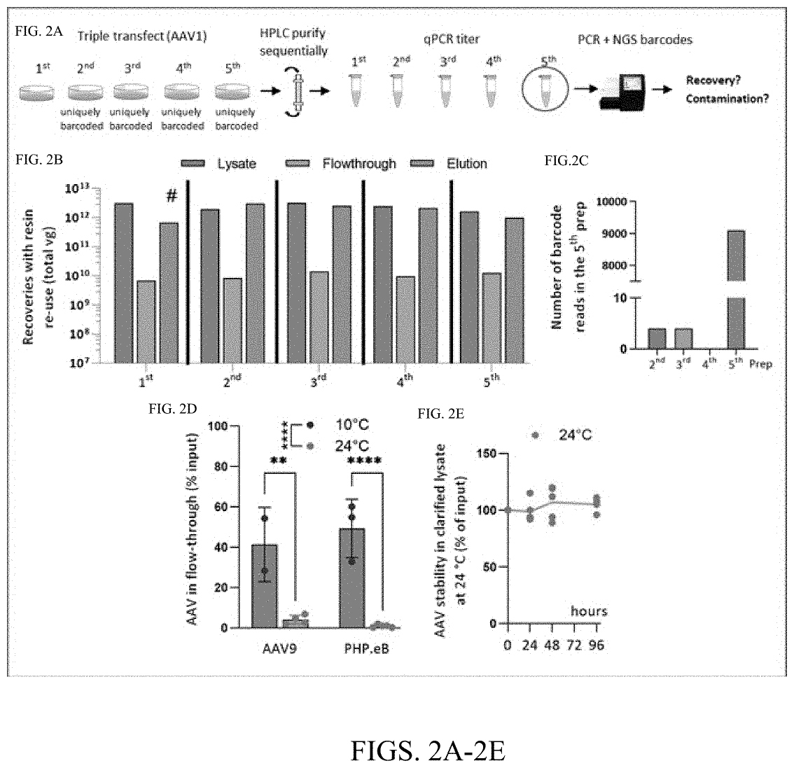

Next, we aimed to determine whether HPLC with AAVX can also be used to purify small-scale preps, and whether resin can be re-used multiple times without contamination or loss of efficiency. Re-using resin is of interest because it decreases the cost and labour associated with AAV purification, and allows automatic back-to-back purification of multiple preps. We produced five different AAV1 preps at small scale, whereby the vectors of the 2^{nd} to 5th AAV1 prep were identical except for a unique 100 bp DNA barcode region. We purified the preps consecutively from prep 1 to prep 5 using the same batch of resin, which was regenerated using 6M guanidine and TBS and 20% ethanol washes between runs. We then quantified the viral vector genomes in the input lysate, the flow-through and final elution via qPCR (FIG. 2A). Throughout the experiment, most of the input virus was found in the elution fraction (<2% found in flow-through) and there was no detectable loss of purification efficiency (FIG. 2B). Furthermore, NGS sequencing of the barcode region in the 5^{th} prep showed that the majority (99.93%) of genomes found in the elution fraction came from the correct 5th prep, not preps 2-4. This indicates that resin can be re-used multiple times without significant loss of efficiency or carryover contamination.

Using the same method as above, we also asked whether the addition of Pluronic F-68 to HPLC buffers increases purification efficiencies. Pluronic F-68 is a surfactant that has been shown to decrease AAV binding to surfaces including plasticware^{24,25}. As HPLC contains long and narrow plastic tubing, we reasoned that addition of Pluronic F-68 may increase purification efficiency by reducing AAV binding to plastic. To test this hypothesis, we added Pluronic F-68 to HPLC buffers to the concentration of 0.1% vol/vol and repeated the experiment described in FIG. 2A (see Fig. S2A). The results indicate that Pluronic F-68 did not increase elution efficiencies for AAV1, although it showed a trend towards increased efficiencies at the post-elution purification steps (Fig. S2B-C) and did not increase carry-over contamination (Fig. S2D-E). This indicates that Pluronic F-68 is a safe addition to HPLC buffers and may be considered for serotypes that are known to be strongly affected by binding to plastic.

### Purification Efficiency is Temperature-Dependent

We also aimed to determine whether purification is affected by temperature, as HPLC machines are commonly housed and run at 4° C. or 10° C. to improve protein stability. We found that decreasing temperature from room temperature (24° C.) to 10° C. increases the fraction of input AAV in the flow-through from less than 5% to 40%-50% for AAV9 and PHP.eB (FIG. 2D). Because the major reason to perform purifications at cold temperatures is to increase AAV stability, we tested whether storage of AAV in clarified lysate at room temperature decreases subsequent AAV titers. qPCR titration of the resulting preps indicates that titers are maintained up to at least 4 days at room temperature, well beyond the average timeline of purification (FIG. 2E). These results indicate that decreasing purification temperature does not improve AAV stability but may result in decreased purification efficiencies.

### An Optimized Purification Protocol

Next, we asked whether AAVX affinity chromatographic purification of AAV can be scaled to higher amounts of input virus (10¹⁴ vg or above). Our initial experiments demonstrated that this was feasible, however those results also indicated that applying for large scale purification the same process as that used for lower amounts of input virus (FIG. 2A) produced unacceptable losses at various steps, particularly at the filter sterilization and buffer exchange steps (not shown). We therefore aimed to systematically reduce vector loss at every step of the process, with the goal of producing a high efficiency protocol independent of the serotype purified. This resulted in a process with the following components (see Supplementary Protocol 1 for process details):

1) In situ lysis using detergents and nucleases. Based on the protocol described by Florencio et al²⁶ and our own observations, in situ lysis using detergents and nucleases is as efficient as separate lysis of the cell pellet, and may be more efficient than in situ lysis using hypertonic salt. To obtain one-step lysis and DNAse removal, we add RNAseA (4.4 μg/ml), Turbonuclease (2.5 U/mL), Triton-X (0.5% vol/vol) and Pluronic F-68 (0.001% vol/vol) to the hyperflask and incubate for 1 hour at 150 rpm shaking, 37° C. to aid lysis with mechanical forces (see Supplementary Protocol 1 for details). Here, Triton-X and RNAseA act as primary lysis agents, Turbonuclease acts to degrade plasmid DNA, while Pluronic F-68 serves to decrease potential AAV binding to plastics.

2) Addition of Pluronic F-68 to all buffers. Based on our observation that the addition of Pluronic F-68 does not reduce HPLC purification efficiencies (Fig. S2), and based on multiple anecdotal sources indicating that the coating of plastic and/or filter surfaces with surfactants may reduce protein binding, we add Pluronic F-68 at 0.01% vol/vol concentration to the elution buffer and incubate all plasticware that comes into contact with AAV with a Pluronic F-68 containing solution (FFB: 1×PBS, 172 mM NaCl, 0.001% Pluronic F-68) for approximately 15 min at room temperature. Additionally, pipette tips and serological pipettes are also coated prior to handling AAV.

3) Stringent resin cleaning with 0.1M phosphoric acid and 6M guanidine. While we observed no loss in AAV binding efficiencies with resin re-use at small scales with AAV1 (FIG. 2, S2), we did observe some loss of binding efficiencies with re-use at large scales, particularly for PHP.eB (data not shown). Based on the recommendations of the AAVX manufacturers (Alejandro Becerra, Thermo Fisher Scientific, personal communication), we increased resin cleaning stringency from 5 minutes with 6M guanidine alone to 15 minutes with pH 10.1M phosphoric acid followed by 15 minutes 6M guanidine HCl. These changes restored efficient resin binding up to at least 6 resin re-uses for both AAV9 and PHP.eB (Fig. S3A,B) with no increase in AAV in flow-through observed (Fig. S3C).

4) Careful buffer exchange. Our analysis indicated substantial losses at the buffer exchange step (25%-50%—not shown). This can be caused by AAV binding to plastic/filter surfaces or overconcentration on the filter surface during buffer exchange, leading to sedimentation of AAV. To mitigate loss of AAV due to binding, we pre-treated all filters/plasticware with Pluronic F-68 as described above. To reduce vector loss due to overconcentration and precipitation, we switched to Amicon Stirred Cell concentrators, which allow for use of higher volumes and continuous mixing during concentration. Alternatively, we use Amicon Ultracel 15 concentrators with frequent (every 2 minutes of centrifugation) mixing and washing of the filter and do not exceed approximately 2×10¹³ vg of AAV per one concentrator.

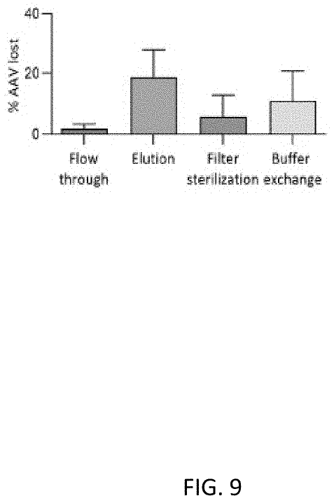

The resulting process is summarized in FIG. 3A. qPCR analysis of the amount of AAV found in different fractions of the optimized process indicate high recovery efficiencies at every step, with an overall average purification efficiency of approximately 75% for AAV9 and approximately 65% for PHP.eB (FIG. 3B). This improved recovery is driven by a considerable increase in efficiency at the filter sterilization+buffer exchange steps compared to the non-optimized protocol (FIG. 3C) and leads to an increased overall purification efficiency (FIG. 3D). Using. This optimized protocol, we obtained an average yield of 2×10¹³ vg per hyperflask across multiple different vectors (FIG. 3E; analysis includes some vectors with transgenes that have lower than average production yields). Analysis of AAV loss at each step indicates that less than 5% of AAV is lost to the flow-through or at the filter sterilization step, while 10% and 20% on average are lost at the buffer exchange and elution steps respectively (FIG. 9), indicating potential targets for future optimization.

### The Purity, Yield and Bioactivity of AAVX-HPLC Purified AAV is Comparable to Iodixanol Purified AAV

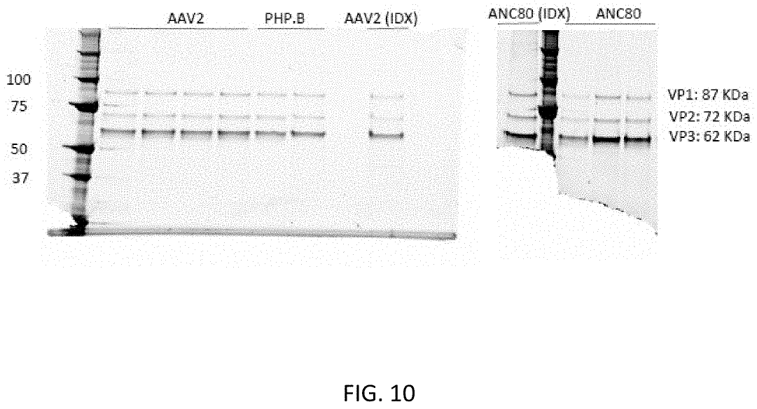

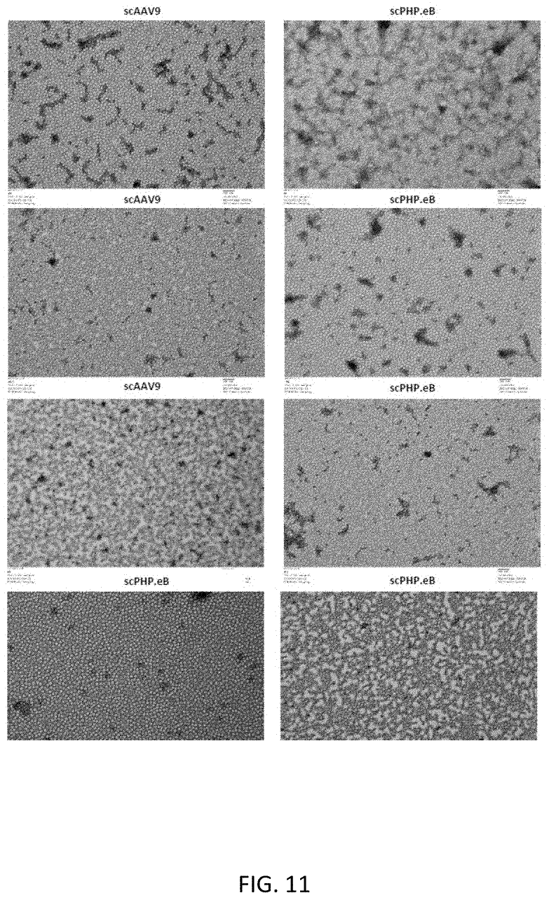

To determine whether HPLC purified virus is qualitatively and quantitatively comparable to iodixanol ultracentrifugation purified virus, we compared HPLC purified virus and iodixanol purified virus on purity, empty capsid content, in vitro bioactivity and in vivo bioactivity. Analysis by gel electrophoresis indicates that HPLC purified preps are comparable to iodixanol purified preps and consist mainly of the expected VP1-VP3 bands, with little-to-no unspecific bands present (FIG. 4A, FIG. 10). Furthermore, in vitro infectivity assay of HEK 293 cells indicates that HPLC and iodixanol purified viruses are equally efficient at infecting cells in vitro (FIG. 4B). Negative stain transmission electron microscopy of the HPLC purified preps indicate an average of approximately 30% empty capsids (FIG. 4C, FIG. 11), whereas approximately 20% empty capsids are commonly reported for iodixanol ultracentrifugation purified AAV²⁷ (also see Discussion).

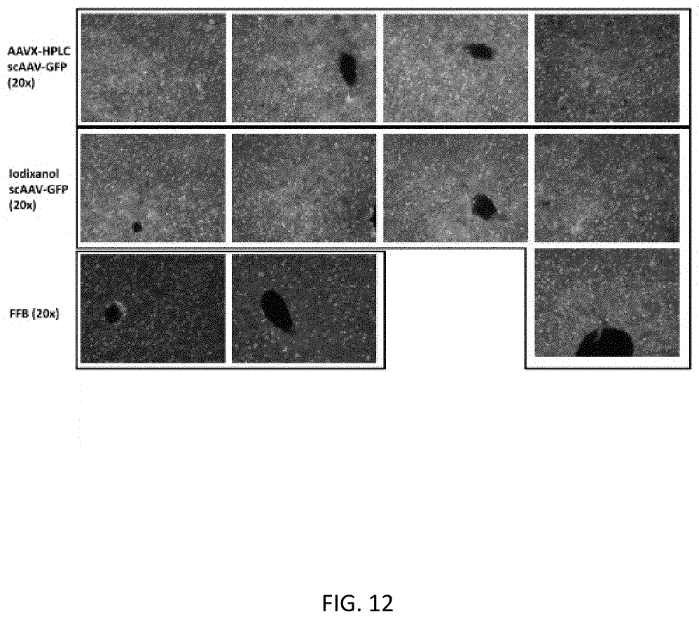

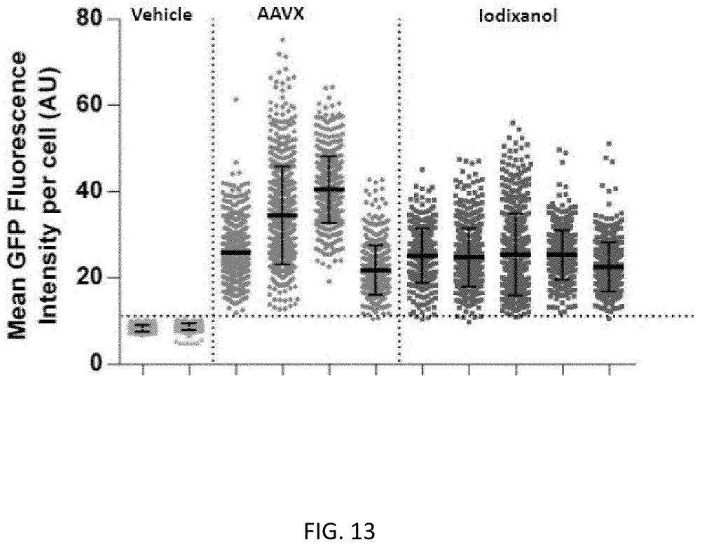

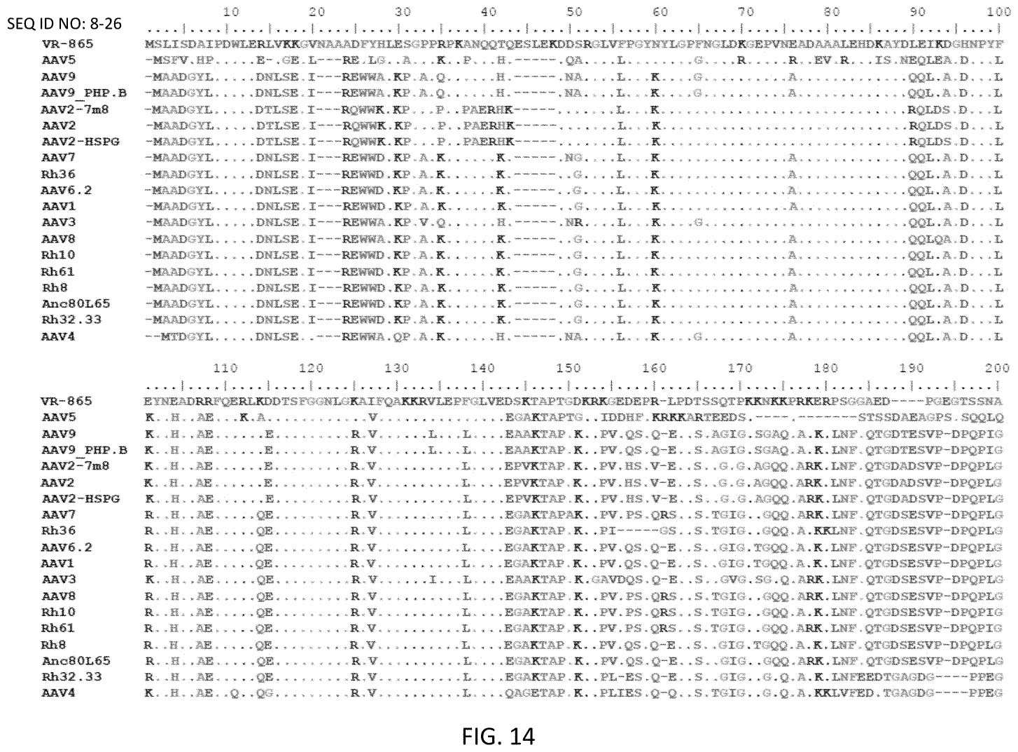

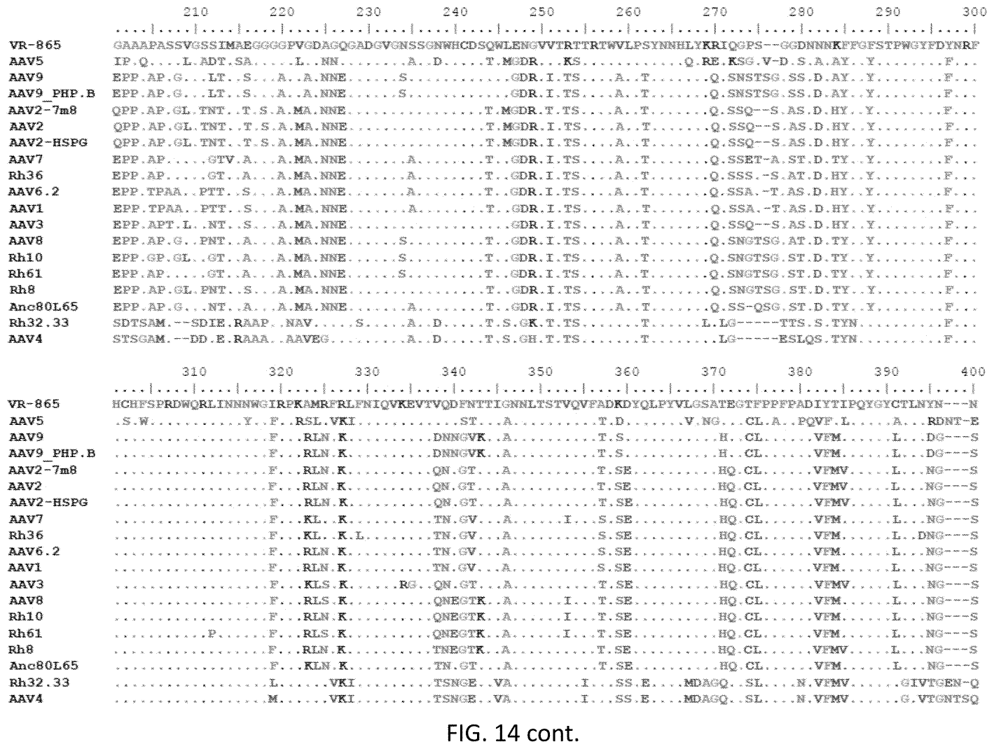

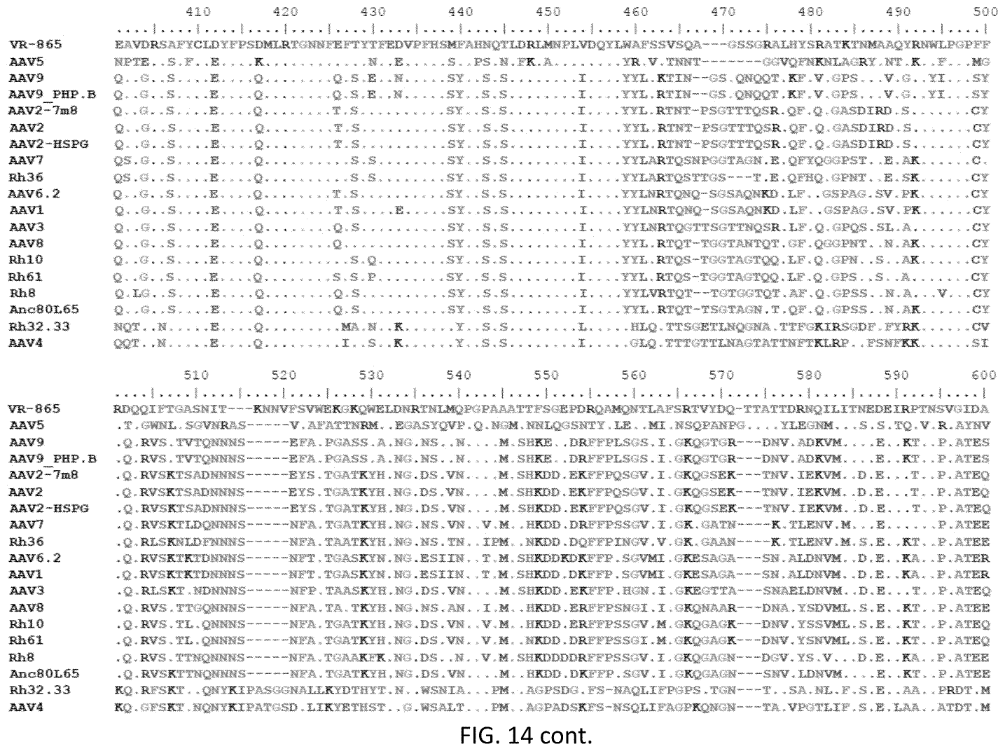

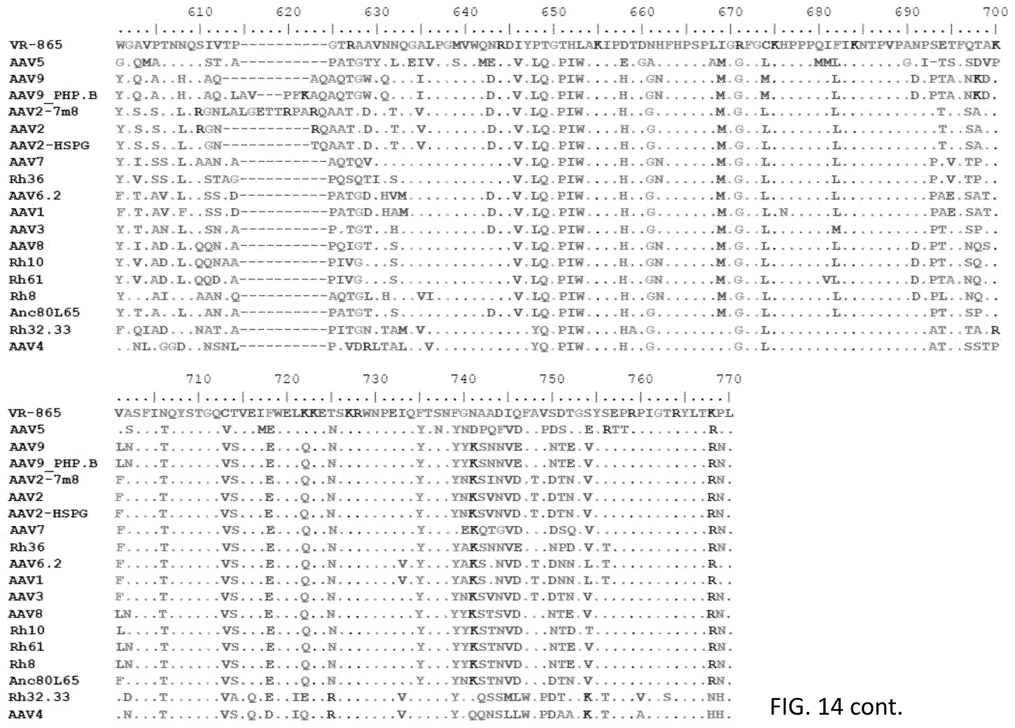

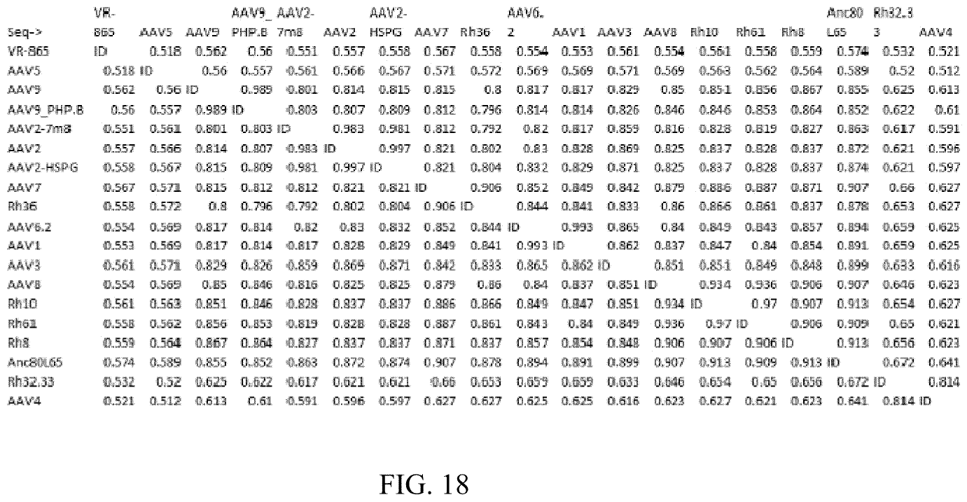

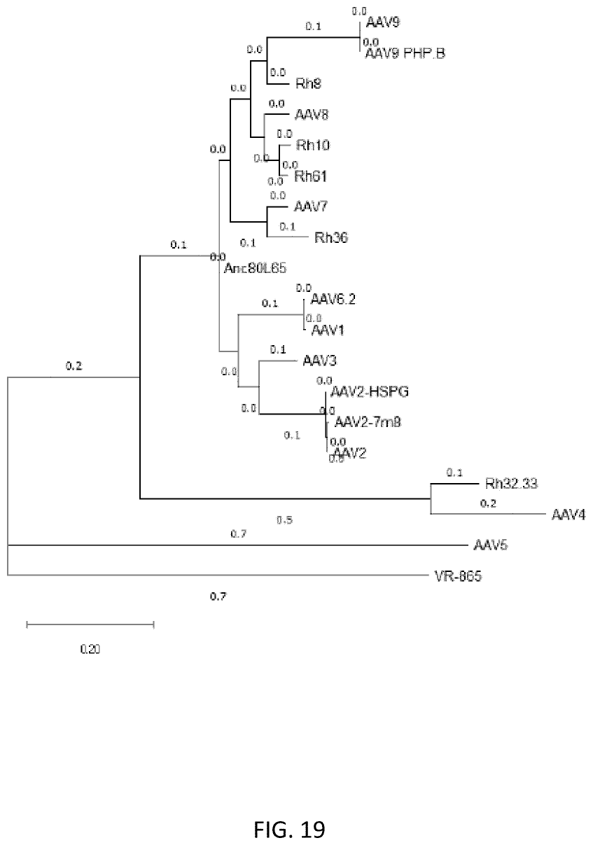

Finally, we compared in vivo bioactivity of HPLC and iodixanol purified viruses. We injected a total of 10¹¹ vector genomes of self-complementary AAV9 carrying a Cbh-EGFP expression cassette retro-orbitally into 6-week-old wild-type male C57BL/6J mice. We euthanized mice 4 weeks post-injection and assayed AAV DNA levels and biodistribution as well as GFP expression in liver, quadriceps and brain. Transgene DNA, RNA and protein levels did not significantly differ between AAVX-HPLC and iodixanol purified viruses for any tissues (FIG. 5A). To confirm this observation, we sectioned, stained and imaged livers of injected mice (FIG. 5B). Image analysis indicates that GFP mean fluorescence intensity does not differ significantly between animals injected with AAVX-HPLC and iodixanol purified viruses, and that viruses purified with both methods transduced almost 100% of liver cells (FIG. 5C-D and FIG. 12-13). Taken together, these data indicate that AAVX-HPLC purified AAV is comparable in purity and bioactivity to iodixanol ultracentrifugation purified AAV.

### AAV Production and Purification

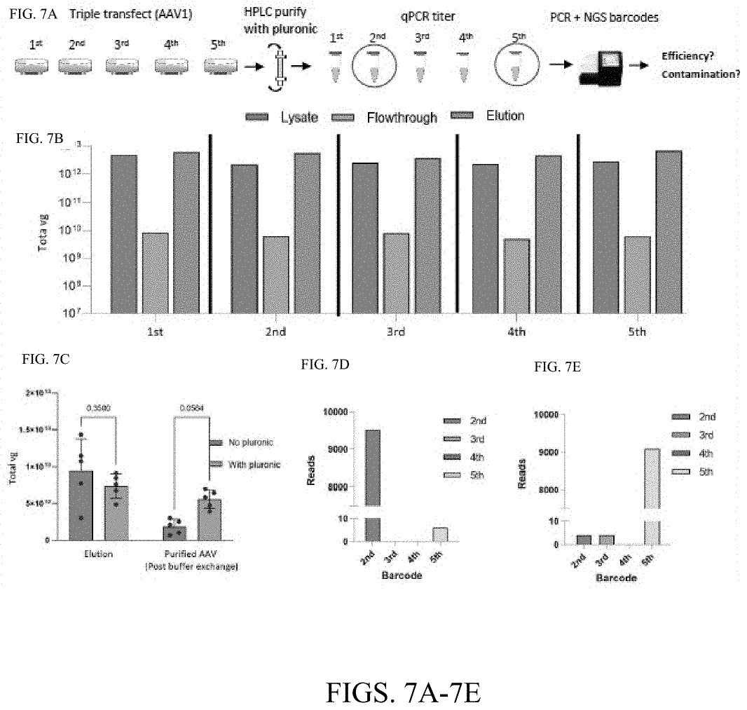

All AAV vectors were produced in HEK293 cells via the triple plasmid transient transfection method as described previously 6. For small scale preps (FIG. 3 and FIG. 7), HEK293 cells were seeded in 15 cm dishes and grown to 80% confluency in DMEM containing 10% FBS (26140079 Gibco) and 1% PenStrep (15140122 Thermo Fisher). Cells were then triple transfected with the vector, AAV1 rep/cap (Addgene 112862), and Ad helper plasmid (pAd delta F6 from UPENN) at a ratio of 1:1:2 (13 μg:13 μg:26 μg per 15 cm dish) using PEI Max 40000, pH 7.1 (24765-1 Polysciences, Inc.) at a ratio 1.375:1 of PEI:total DNA. Cells were harvested 3 days post-transfection by scraping cells off the plate in their conditioned media and lysing cells through 3× freeze-thaw cycles between 37° C. and −80° C. Preps from 3 replicate plates were then pooled, incubated with 25 U/ml of benzonase (E8263-25KU Millipore(Sigma)) at 37° C. for 1 hour to remove plasmid DNA, centrifuged at 4° C., 4000 g for 30 min and supernatant filtered through 0.22 μm PES bottle top filter (431097 Corning). The filtered lysate was then split into two equal parts, with one part purified using standard HPLC purification reagents and the other part purified using reagents containing 0.1% vol/vol Pluronic F-68 (24040032 Thermo Fisher) (Described in FIG. 3 and Fig. S2 respectively).

For hyperflask scale preps described in FIG. 2, HEK293 cells at 80% confluency from four 15 cm dishes were seeded to a hyperflask (CLS10031-4EA Millipore Sigma), grown to 80% confluency and triple-transfected with AAV vector, Rep/Cap for AAV2, Anc80, PHP.eB, or AAV9 (AAV2: 104963 Addgene; Anc80: 16, PHP.eB: 103005 Addgene, AAV9: 112865 Addgene) and pAdΔF6 at 130m:130m:260m per hyperflask respectively. Three days after transfection, clarified harvests (560 ml) were treated with 12500 total units of benzonase (E8263-25KU Millipore (Sigma)) for 30 minutes at 37° C. and this step was repeated with an additional 2500 total Units of benzonase for one more hour at 37° C. to remove plasmid DNA. The harvest was precipitated overnight at 4° C. in high salt solution (80 ml of 5M NaCl). The clarified lysate was obtained by centrifugation at 10000 rpm for 30 minutes at 4° C. The supernatant was collected and filtered using 0.22 μm PES filter unit before HPLC purification.

### High-Efficiency Purification Protocol

For hyperscale preps described in FIG. 4, an optimized protocol based on Florencio et al²⁶ and our own observations was used. HEK293 cells at 80% confluency from four 15 cm dishes were seeded to a hyperflask, grown to 80% confluency (normally approximately 48 hours after seeding) and triple-transfected with AAV vector, Rep/Cap for AAV9 or PHP.eB and pAdΔF6 at 130m:130m:260m per hyperflask respectively. Four days post-transfection, supernatant from a hyperflask was decanted into a 1 liter flask and 3 ml Triton-X 100 (8787-100ML Millipore Sigma), 2.5 mg RNAse A at 1 mg/ml concentration (10109142001 Millipore Sigma), 25 U/ml of Turbonuclease (ACGC80007 VitaScientific) and 56 μl of 10% Pluronic F-68 (24040032 Thermo Fisher) was added to the supernatant. The supernatant was then mixed, poured back into the hyperflask, and shaken on an orbital shaker at 150 rpm at 37° C. for 1 hour to lyse the cells and remove plasmid DNA. Lysate was then decanted from the hyperflask, and the hyperflask washed with 140 ml of DPBS (10010072 Life Tech) which was added to the rest of the lysate. The total lysate was then centrifuged at 4000 g, 4° C. for 30 min, and the supernatant was filtered through a 0.45 μm PES bottle-top filter (295-4545 Thermo Fisher) before loading onto HPLC.

Iodixanol-ultracentrifugation purified preps were produced in the Gene Transfer Vector Core at Schepens Eye Research Institute. HEK 293 cells were seeded and transfected into hyperflasks, followed by benzonase (E8263 Sigma-Aldrich) treatment and high salt lysis as described above, then lysate clarification, concentration of the lysate using tangential-flow filtration, iodixanol gradient ultracentrifugation and buffer exchange for FFB (Final Formulation Buffer: 1×PBS, 172 mM NaCl, 0.001% Pluronic F-68).

### High Performance Liquid Chromatography

AAV purification was performed using AAVX POROS CaptureSelect (ThermoFisher Scientific) resin bought as pre-packed 1 ml columns (Thermo Fisher A36652) or resin (Thermo Fisher A36741) with 6.6 mm×100 mm column (Glass, Omnifit, kinesis-USA) in an AKTA Pure 25 liter HPLC system (29018224 GE Life Sciences) containing an auxiliary sample pump S9 (29027745 GE LifeSciences). The machine was setup at room temperature and all purifications were performed at room temperature (approximately 24° C.), except for experiments described in FIG. 3D. Column volume [CV] for each purification was set as 1 ml regardless of the actual volume of resin used. For purifications using more than 1 ml of resin, a protocol with increased wash times was employed (see Supplementary Files 1, 2). The chromatography column was pre-equilibrated with 10 [CV] of wash buffer 1×Tris-buffered Saline (1×TBS) (Boston Bioproducts), before application of AAV lysate. Equilibration and all subsequent washes of the column were performed at a rate of 2 ml/minute.

Lysate was clarified at most 1 day prior to loading onto the HPLC and warmed up to room temperature prior to loading. Lysate was loaded at a flow rate to resin volume ratio ensuring approximately 2 minute residence time in the resin, normally using 1 ml of resin (AAVX and a flow rate of 0.5 ml/min, or 4 ml resin with a flow rate of 2 ml/min. At least 1 ml of resin per one hyperflask was used; if preps from multiple hyperflasks were pooled, the volume of resin was increased accordingly.

For purifications using 1 ml of resin, the column containing bound AAV was then washed with 10 [CV] of 1×TBS, followed by washes of 5 [CV] of 2×TBS, 10 [CV] 20% ethanol and 10 [CV] 1×TBS wash. The bound AAV was eluted using a low-pH (pH 2.5 to 2.9) buffer of 0.2 M Glycine in 1×TBS, containing 0.1% vol/vol Pluronic F-68 at a rate of 1 ml/minute. Resin was then washed with 10 [CV] of 1×TBS regenerated with 15 [CV] 0.1M pH 1 phosphoric acid and 15 [CV] 6 M guanidine HCl at flow rate of 1 ml/min, and washed again with 10 [CV] 20% ethanol and 10 [CV] 1×TBS. Elution fractions were taken as 1 ml volumes per fraction. The eluted virus solution was neutralized by adding 1 M Tris-HCL (pH 8.0) at 1/10^{th} of the fraction volume directly into the fraction collection tube prior to elution. Peak fractions based on UV (280 nm) absorption graphs were collected, filter sterilized using 0.2 μm PES syringe filters (Corning 431229), buffer exchanged using either Amicon Ultracel (Merck Millipore UFC910008) or Amicon Stirred Cell (Merck Millipore UFSC05001) concentrators with a molecular weight cut-off of 50 kDa or 100 kDa (UFC905008 EMD Millipore) prior to virus titration. For Amicon Stirred Cell concentrator, high-purity nitrogen gas (NI UHP80 Airgas) was used at 40-70 psi as a pressure source. All plasticware and tips were coated or incubated with FFB for approximately 15 minutes at room temperature prior to applying AAV containing solutions at any step of the purification process.

### Quantitative PCR and Digital Droplet PCR

In brief, genomic titer was determined by a quantitative PCR (TaqMan, Life Technologies) as well as digital droplet PCR (ddPCR). For qPCR, real-time qPCR (7500 Real-Time PCR System; Applied Biosystems, Foster City, CA, USA) with EGFP-targeted primer-probes (AGCAAAGACCCCAACGAGAA (SEQ ID NO: 1), GGCGGCGGTCACGAA (SEQ ID NO: 2), 6FAM-CGCGATCACATGGTCCTGCTGG-TAMRA (SEQ ID NO: 3)) was used. Linearized CBA-EGFP DNA at a series of dilutions of known concentration as a standard was used. After 95° C. holding stage for 10 seconds, two-step PCR cycling stage was performed at 95° C. for 5 seconds, followed by 60° C. for 5 seconds for 40 cycles. Genomic vector titers were interpolated from the standard. qPCR was used to determine titers for experiments described in FIGS. 1, 2, 3 and S2.

For ddPCR, QX200 ddPCR system (Biorad) using the same EGFP-targeted primers-probes were used as above. ddPCR and titer estimation was performed as previously described in Sanmiguel et al.⁴⁰. ddPCR was used to estimate titers for experiments described in FIG. 4, FIG. 5 and FIG. 9-11.

### Protein Gel Analysis

All materials and reagents used were purchased from Life Technologies. Equal vector genome of AAV were loaded on a NUPAGE 4-12% Bis-Tris polyacrylamide gel (Life Technologies, Grand Island, NJ) and subjected to electrophoresis at 150 V for 1 h and 30 min. For each AAV preparation, a volume corresponding to a titer of 1010 vg was mixed with 5 μl 4×NuPAGE lithium dodecyl sulfate (LDS) sample buffer and 1×PBS (21-031-CM, Corning) to 20 μl total volume and heat denatured at 70° C. for 5 min.

SYPRO Ruby Protein Gel Stain (ThermoFisher Scientific, Waltham, MA, USA) was applied per the manufacturer's protocol to visualize and analyse SDS-PAGE bands. In brief, the gel was fixed in 7% Glacial Acetic Acid, 50% methanol (ACS grade, Fisher Scientific) in ultra-pure water, for 15 minutes at 21° C. (room temperature) by gentle agitation. Fixation was repeated once more before gel was rinsed with ultra-pure water. Gel was stained with SYPRO Ruby as follows: 30 seconds microwave, 30 seconds agitation, 30 seconds microwave, 5 minutes agitation, 30 seconds microwave, 23 minutes agitation. Gel was rinsed with ultra-pure water and destained with 7% Glacial Acetic Acid, 10% methanol for 30 minutes at 21° C. (room temperature) by gentle agitation. Proteins stained with the dye were visualized with a 302 nm UV transilluminator (ChemiDoc XRS+Biorad).

### Empty Capsid Estimation Via Transmission Electron Microscopy

Purified and formulated AAV from different preps was diluted to 10¹³ vg/ml and submitted for negative stain and transmission electron microscopy analysis at Harvard Medical School Electron Microscopy Core. In short, the sample is diluted in water and adsorbed onto a glow-discharged carbon or formvar/carbon coated grid. Once the specimen has been adsorbed on to the film surface, the excess liquid is blotted off using a filter paper (Whatman #1) and the grid is floated on a small drop (˜5 μl) of staining solution (most commonly 0.75% uranyl formate, 1% uranyl acetate or 1-2% PTA). After 20 seconds the excess stain is blotted off and the sample is air dried briefly before it is examined in the transmission electron microscope. At least two images were taken per prep at 30,000× magnification, and at least 200 virions were counted manually per image by two researchers blinded to the identity of the image, and empty and full ratios averaged between resulting counts. Due to the difficulty in confidently differentiating full and partially filled capsids using electron micrographs, virions were counted as empty and full only based on the criteria described in 39. On the minority of cases where a virion could not be confidently assigned to either (<1% capsids) the virion was not counted. Similarly, virions were not counted in areas of images, with image noise, artefacts, clumping, or other effects that obscure a clear classification of the virion type.

### Next Generation Sequencing and Analysis

For FIGS. 2B and 12B, five different AAV1 preps were produced, where the vectors from the 2^{nd} to 5^{th} prep were identical except a unique DNA barcode region. The preps were purified consecutively from prep 1 to prep 5, and the barcode region was PCR amplified in the elution fractions of the 5^{th} preps. The amplicons were PCR amplified and submitted for Amplicon Seq at the MGH DNA Sequencing core. Finally, the number of barcodes corresponding to AAVs from each of the preps 2-5 were directly counted from the resulting FASTQ file. The vast majority of barcodes present came from the 5^{th} preps (barcodes from previous preps were present at less than 0.1%).

### AAV In Vitro Studies

HEK293 cells were seeded at 2×10⁵ cells/well and infected 24 hr later, in triplicate, with AAV at an MOI (multiplicity of infection) of 10³ or 10⁵ vg/cell in a 500-μl volume for AAV2 and Anc80 respectively. The media was replaced 24 hr post-infection with 1 ml of complete DMEM containing 10% (vol/vol) heat-inactivated fetal bovine serum (FBS), and 100 U/ml penicillin and 100 μg/ml streptomycin. Cells were then imaged using BioRad imaging system.

### AAV In Vivo Studies

All animal procedures were performed with the approval of the Institutional Animal Care and Use Committee (IACUC) at Schepens Eye Research Institute. For assaying in vivo potency and transduction, self-complementary AAV9 carrying a Cbh-EGFP expression cassette was produced at hyperflask scale and purified with AAVX-HPLC or iodixanol ultracentrifugation, concentrated in FFB and stored at −80° C. until use. 6-week old male C57BL/6J mice were then injected retro-orbitally with a total dose of 10¹¹ vector genomes (in 100 μl volume of FFB) per mouse. Mice were euthanized 4 weeks post-injection and brain, quadriceps and liver harvested. One part of each tissue was snap frozen in liquid nitrogen for analysis of viral DNA and GFP RNA and protein (see below). Another part of each tissue was fixed in PFA for later sectioning (see Imaging and image analysis).

### DNA and RNA Quantification

Tissues were homogenized by disrupting 30 mg of tissue in 1 ml of RLT+buffer for DNA and RNA and 1 ml of RIPA buffer containing 1×Halt protease and phosphatase inhibitors for protein (78444 Thermo Fisher Sci). For disruption, samples, buffer and 1 mm Zirconia/Silica beads (11079110z Biospec) were loaded into XXtuff vials (330TX BioSpec) and disrupted using Mini Beadbeater 24 (112011 BioSpec) at max speed for 3 minutes. Vials were then placed on ice for 2-5 minutes for RNA and 1 hour for protein, centrifuged at 10 000 g for 3 min and supernatant used for further procedures.

For DNA/RNA, 700 μl of supernatant was loaded onto AllPrep DNA Mini Spin Columns and purified using AllPrep DNA/RNA/miRNA Universal Kit (80224 Qiagen) for quadriceps and Allprep DNA/RNA mini kit (80204 Qiagen) for brain and liver. Purification was performed on Qiacube Connect (9002864 Qiagen).

Total AAV copy number was assessed using GFP primers and linearized CBA-GFP plasmid dilution series as standard for AAV copy number (AGCAAAGACCCCAACGAGAA (SEQ ID NO: 1), GGCGGCGGTCACGAA (SEQ ID NO: 2), 6FAM-CGCGATCACATGGTCCTGCTGG-TAMRA (SEQ ID NO: 3)). Total genome copy number was estimated using RPII primers-probes (GTTTTCATCACTGTTCATGATGC (SEQ ID NO: 4), TCATGGGCATTACTATTCCTAC (SEQ ID NO: 5), probe: VIC-AGGACCAGCTTCTCTGCATTATCATCGTTGAAGAT-3IABkFQ (SEQ ID NO: 6)) along with a standard of gDNA dilution series of known concentration. AAV copy number per diploid genome was then calculated as

$$copy number per diploid genome = 2 * ( \frac{total AAV copy number}{total genome copy number} ) .$$

Efficiency and specificity of amplification for both primer-probe sets was previously established, and amplification was performed using Luna Universal Probe qPCR Master Mix (M3004 L NEB) at thermocycling conditions recommended by the manufacturer.

For quantification of GFP RNA expression, RNA extracted from tissues was first treated with DNAse (DNA-Free™ DNA Removal Kit AM1906, Thermo Fisher) and then reverse transcribed and amplified using Luna Universal Probe One-Step RT-qPCR Kit (E3006 L NEB) according to manufacturer's instructions. Primer-probe sets for GFP RNA (AGCAAAGACCCCAACGAGAA (SEQ ID NO: 1), GGCGGCGGTCACGAA (SEQ ID NO: 2), 6FAM-CGCGATCACATGGTCCTGCTGG-TAMRA (SEQ ID NO: 3)) and RPII RNA (GTTTTCATCACTGTTCATGATGC (SEQ ID NO: 4), AATCAATGCAGGTTTTGGCGATG (SEQ ID NO: 7), probe: VIC-AGGACCAGCTTCTCTGCATTATCATCGTTGAAGAT-3IABkFQ (SEQ ID NO: 6)) were used. Controls lacking reverse transcriptase were ran to preclude signal from DNA contamination. Expression of GFP RNA normalized to RPII RNA was then calculated as 2^{-(Ct}^{_{gFP}}^{-Ct}^{_{RPII)}}.

For quantification of GFP protein expression, protein lysate was first diluted 5× twice in fresh RIPA+Halt inhibitors buffer and all dilutions were assayed for total protein content using Pierce™ BCA Protein Assay Kit (23225 Thermo Fisher). For each tissue type, lysates were then diluted in RIPA+Halt inhibitors buffer to the concentration where they would be at the lower end of the linear range for GFP quantification using anti-GFP antibody ab290 (ab290 Abcam) on Wes (Protein Simple). Linear range for GFP quantification was previously determined by assaying GFP using 12-230 kDa Jess or Wes Separation Module (SM-W004 Protein Simple) on Wes with ab290 for dilutions ranging from 3 μg/μl to 0.03 μg/μl (linear range: liver <0.3 μg/μl, brain 0.3 μg/μl to 1.5 μg/μl, quadriceps 0.03 μg/μl to 1.3 μg/μl). Linear range for total protein was also previously determined by assaying total protein in the range of 4 μg/μl- to 0.1 μg/μl using Total Protein Detection Module (DM-TP01 Protein Simple) (linear range: <1 μg/μl for all tissues tested). GFP and total protein levels were then assayed and GFP and total protein quantified using Compass for SW 4.1 (Protein Simple). Finally, GFP was normalized to total protein to arrive at the final value.

### Immunofluorescence and Image Analysis

Tissues were fixed in 1% PFA for 4 hours and then 4% PFA for 1 hour at room temperature (21° C.). Fixed tissues were then washed with 1×PBS three times for 5 min, placed in 30% sucrose for approximately 48 hours at 4° C., and frozen in OCT blocks by submersion into isopentane cooled by liquid nitrogen. Blocks were then sectioned at 12 μm thickness using iHisto cryosectioning service (iHisto, Inc.). Sections were kept at −80° C. until staining. Sections were blocked using Blocking buffer (10% Normal Goat Serum (NGS), 2% Bovine Serum Albumin (BSA), 0.1% Tween-20) for 1 hour, washed 3×5 min with PBS-T (PBS+0.1% Tween-20), stained with Tomato lectin at 10 μg/ml (DL-1177 Vector Laboratories) for 1 hour, washed 3×5 min with PBS-T, stained with DAPI for 5 min at 1:1000 stock concentration (D1306 Thermo Fisher), mounted for 15 minutes (H-1400 Vector Laboratories) and imaged for native GFP, tomato lectin and DAPI. All actions were performed at 21° C. in a dark room. Slides were imaged using a Zeiss Axio Observer D1 microscope (exposure times were set such that signal intensities from samples with the brightest signals would appear in the lower third of the histogram). Exposures were kept constant between all samples for all three colours imaged. For each tissue, two sections from the middle of the tissue were imaged, with 6-8 fields total imaged at 200× magnification.

Three images of different sites were then selected, all cells within the images circled for regions of interest (ROIs) and cell GFP mean fluorescence intensity quantified within ROIs in Fiji 41. Cells were circled conservatively to make sure only individual cells were circled. A total of 400-700 cells were quantified per animal, and mean fluorescence intensity values across different cells averaged to arrive at an overall liver GFP mean fluorescence intensity per animal.

### AAV Phylogenetic Analysis

To generate the phylogeny, first 19 representative AAV capsids were chosen, including an avian AAV (VR-865) for use as an outgroup for eventual tree rooting. The VP1 amino acid sequences from all of these different isolates were aligned through ClustalOmega⁴² as implemented on the EMBL-EBI webserver⁴³. Substitutions models and parameters for an eventual maximum likelihood (ML) phylogenic analysis were evaluated by ProtTest3⁴⁴, and the best fitting model by the Aikake Information Criterion (AIC) was selected. The model best describing the set of AAV sequences was the Le & Gascuel model⁴⁵, with a discrete Gamma distribution (5 categories) to model rate differences among sites within the alignment. This model was used to construct a ML phylogeny through MEGA X⁴⁶, before being exported and visualized through phytools⁴⁷. See Supplementary FIGS. S9-S11 for multiple sequence alignment, sequence percent identity and Newick formatted phylogeny of the phylogeny depicted on FIG. 1B.

### Statistical Analysis

All data was visualized, and statistical analysis was performed in GraphPad Prism (GraphPad). Specific statistical tests used are listed in figure legends for each test, and all tests were performed with default settings unless otherwise specified.

### Supplementary Protocol: Production of AAV Using Hyperflasks and AAVX Affinity Chromatography

For 1-4 hyperflask, the protocol takes approximately 10-14 days.
We recommend ordering hyperflasks 2-3 months in advance, as they are commonly heavily backordered.
All DNA used for transfection needs to be endotoxin free; we recommend Qiagen Endo Free kits or outsourcing to endo free companies (PureSyn); we DO NOT recommend using Zymo Endo Free kits due to their high variability in performance.
Transgene plasmid needs to contain intact ITRs: we recommend testing ITR integrity with SmaI/XmaI digests and/or next generation sequencing for each DNA prep.
For buffer exchange, we recommend use of Amicon Stirred Cell concentrators, particularly for purification of PHP.B and related variants, which tend to strongly sediment. Buffer exchange using Amicon Stirred Cell results in more consistently high recovery and low sedimentation. However Amicon Stirred Cell is more time-consuming, not disposable and requires an upfront investment to set up the system; therefore use of Amicon Ultra 15 Centrifugal filter devices can be used with low titer preps (<1×1013), given careful handling (see Buffer exchange and titration).

### Required Items (Per Hyperflask):

Approximately 1 month prior to the start of AAV production, ensure you have all the required reagents (below). Order any that are missing and produce DNA with endo-toxin free kits.

### Tissue Culture and Transfection:

Cap DNA: 130 μg
deltaF6 or other packaging plasmid: 260 μm (UPenn Vector Core)
Transgene DNA: 130 μg
1 L sterile filtered DMEM high glucose with 10% FBS, 1% Penstrep (11965118 Thermo Fisher, 15-140-122 Fisher Scientific)
600 mL of filtered DMEM high glucose with 1% Penstrep without FBS
Low passage HEK293T cells
5×15 cm tissue culture dishes
1× hyperflask (any variant is fine) (CLS10031-4EA Millipore Sigma or others)
715 μg PEIMax (1 mg/ml, pH 2.6, sterile filtered) (24765-1 Polysciences Inc.)

### Lysis and Clarification:

2×1 L bottle-top PES 0.45 μm filters (1143-RLS, Foxx Life Sciences)
3 ml Triton-X 100 (T8787-100ML, Sigma)
2.5 mg RNAse A (1 mg/ml concentration)
56 μl of Turbonuclease (at 250 U/μl starting concentration, to a final of 25 U/mL in the media) (ACGC80008, Vita Scientific)
56 μl of 10% Pluronic F68 (final concentration 0.001%)
A centrifuge that can spin 2×500 ml tubes at 4000 g or more (or spread the media across more tubes)
2×500 ml centrifuge tubes 2

### HPLC:

POROS CaptureSelect AAVX resin (prepacked or free): (A36739 Thermo Fisher, A36652 Thermo Fisher)
AKTA Pure 25 HPLC system with sample pump or equivalent. Housed at room temperature.

### HPLC Buffers:

A1: 2 L 1×TBS (28358 Thermo Fisher)
A2: 1 L 20% EtOH-1×TBS
A3: 1 L 2×TBS
A4: 500 mL 6M Guanidine (SRE0066-100ML, Sigma Aldrich or make from G3272-2KG Sigma Aldrich in distilled water)
A5: 1 L 20% EtOH
A6: 500 mL Phosphoric acid 0.1M, pH1 (PX0996-6 Sigma Aldrich)
B1: 500 mL 0.2M Glycine, pH 2 to pH 2.5, 0.01% vol/vol Pluronic F68 elution buffer
1M Tris pH8, 0.1% vol/vol Pluronic F68 neutralization buffer
20×15 mL tubes
1 L 0.1M NaOH
1 L H2O
1 L EtOH 20%
0.1M and 1M NaCl for packing and column qualification
Final Formulation Buffer (1×PBS, 35 mM NaCl and 0.001% Pluronic F68)—filter sterilize with 0.2 μm filter
NB! Filter all HPLC buffers using a 0.45 μm or 0.2 μm bottle top filters. This is required to avoid introduction of sedimented salts or other particulate matter into the HPLC, which can create clogs in the flow path.
NB! 6M Guanidine and 0.1M Phosphoric acid are hazardous: use proper PPE and precautions in handling and disposal.

### Buffer Exchange:

0.2 μM PES syringe filters (CLS431229-50EA, Sigma Aldrich) with 20-50 mL syringes
For preps <1×1013 vg of AAV: 50 or 100 kDA, Amicon Ultra 15 Centrifugal Filter devices (UFC905008 or UFC910008, EMD Millipore)
For preps >1×1013 vg of AAV:
Ultrafiltration Discs, 100 kDa (NMW PLHK04310 Millipore Sigma)
500 mL of 5% hydrogen peroxide in PBS-filter sterilized. Make this fresh every time, as hydrogen peroxide activity/stability decreases in neutralized pH.
500 mL of 70% ethanol-filter sterilized
500 mL of Final Formulation Buffer-filter sterilized.
Amicon Stirred Cell (UFSC05001OR) with a system for providing sterile nitrogen gas pressure, such as:
NI UHP80 (NITROGEN UHP GR 5.0 SIZE 80 CGA) from Airgas
High purity pressure regulator Yl1N245D580-AG (REGULATOR FIRST STAGE HIGH PURITY 3500/100 BRP DIAMETER VALVE ¼″C CGA580CV)
In line sterilizing filter Y40-LF811P (FILTER ½T 0.003 MICRON 750 PSIG PTFE 10R STAINLESS STEEL)
Sterile magnetic stirrer for use under a tissue culture hood, such as Mini Stirrer (VWR 10153-304) or equivalent
Tube fittings to connect Amicon Stirred Cell to the nitrogen source:
We strongly recommend contacting representatives of Airgas or Swagelok (or equivalent) to verify exact details of the tube fittings and the procedure of safely connecting and operating the nitrogen tank
Stainless Steel Tubing Insert, ¼ in. OD×0.17 in. ID (SS-405-170 Swagelok)
316 Stainless Steel Front Ferrule for ¼ in. (SS-403-1Swagelok)
316 Stainless Steel Back Ferrule for ¼ in. (SS-404-1-Swagelok)
Connect the Amicon Stirred Cell inlet tube to nitrogen outlet valve by:
Inserting stainless steel tubing insert into the tube.
Place the nut-shaped tube fitting onto the tube.
Place front ferrule, then back ferrule onto the tube.
Insert tube into the nitrogen outlet valve (which should be Swagelok pressure fitting).
Using a wrench, screw the nut-shaped tube fitting onto the outlet valve. The front ferrule should displace back ferrule in a manner that compresses the tube securely in place. Test that the tube is tightly secured.
If using the in line sterilizing filter, cut the tube to create two parts, or add an additional ¼ in. tube, then connect to the filter as described above
Catalogue for further reference: https://www.swagelok.com/downloads/webcatalogs/EN/MS-01-140.PDF

### Protocol

### AAV Production

### Cell Seeding

Thaw a vial of low passage HEK293T cells, expand to four 15 cm dishes, at 70-80% confluency in DMEM

### 10% FBS 1% PenStrep;

Filter sterilize all media
Warm all media to 37° C. before use
Do not let cells get more than 90% confluent at any stage during expansion, and seed cells dropwise evenly across the plates+mix gently 10× in a star pattern to ensure that cells are always evenly distributed.
Check that they are not confluent anywhere under microscope the next day and discard any plates that highly confluent on one side and empty on another.
Ideally, passage cells every 48 hours, to avoid acidification of media. 72 h is still okay.
Pool cells from four 15 cm dishes into 560 mL of DMEM 10% FBS 1% PenStrep, mix gently and thoroughly by pipetting to create a homogenous solution with minimal bubbling, pour into hyperflask by placing the hyperflask upright, tilted to the side as per manufacturer's instructions (https://www.youtube.com/watch?v=uo3jEQGz8Z0).
Grow cells until they reach ˜80% confluency. This commonly takes 48 hours. Check that the cells are 70-80% confluent and evenly distributed under a microscope before transfection. Transfecting 100% 4 confluent cells results in ˜3-10× reduced AAV production levels. Transfecting 40-50% confluent cells results in a ˜30-50% decrease in titers.

### Transfection

Mix 10 mL DMEM (sterile filtered, room temp) with DNA (vector:cap:deltaF6 at 130m:130m:260m).
Mix 10 mL DMEM (sterile filtered, room temp) with 715m of PEIMax (1 mg/ml, pH 2.6, sterile filtered).
Mix the two solutions together, shake and vortex immediately for 15 seconds (pulse vortexing, not continuously), incubate at RT for 15 minutes.
Add the solution to 560 ml of DMEM-1% Penstrep (sterile filtered, warmed to 37 C), mix thoroughly and gently with a serological pipette or swirling. NB! Do not include serum here! Inclusion of serum
   in the transfection mix reduces AAV yields by 3-10×.
Remove the hyperflask from the incubator, pour out media carefully so as not to disturb the cells. (https://www.youtube.com/watch?v=1B_3 Luum-ME)
Gently pour the transfection mix in DMEM-1% PenStrep into the hyperflask, top up with DMEM-1% PenStrep and remove bubbles as needed.
Put the hyperflask back into the incubator, incubate for four days. 3-5 days is also fine. Interestingly, the optimal amount of time for incubation varies between different reports, so currently there is no published consensus I am aware of. Anecdotally, 3-5 days results in similar yields.

### Harvesting and Lysis

After 3-5 days of incubation, pour media from the hyperflask into a 1 L bottle.
Add lysis reagents to the media in the bottle:
3 ml Triton-X 100
250 μl of RNAse A (at 10 mg/ml concentration, a total of 2.5 mg RNAse A)
56 μl of 25 U/mL of Turbonuclease
56 μl of 10% Pluronic F68 (final concentration 0.001%)
Mix with a serological pipette until the solution is clear; avoid introducing air or swirling, as it generates foam. Pour media back into the hyperflask slowly (otherwise generates foam). Store any volume that is left over in a 50 mL tube.
The above is necessary, because adding lysis reagents directly into the hyperflask does not allow them to be rapidly uniformly distributed across cells.
Incubate the hyperflask at 150 rpm shaking for 30 min-1 hour at 37° C. along with left-over volume in the 50 mL tube. Shaking incubation aids with mechanical lysis of cells.
If you do not have access to a sterile shaking incubator, we recommend hand shaking for ˜5 min and subsequent incubation at 37° C. or carefully double bagging the hyperflask and incubating in a non-sterile incubator, then disinfecting the outer bags with bleach and ethanol prior to proceeding.
Decant lysate in the hyperflask into a 1 L bottle, wash the hyperflask with 140 mL of PBS and add it to rest of the lysate—forming 700 mL total.
If not proceeding to the HPLC purification on the same day, store at 4° C. (1-2 days) or −20° C.
Clarify the lysate (below). Clarification is critical to prevent clogging of the HPLC during the run. We recommend performing this on the day of loading onto the HPLC, to minimize re-formation of aggregates during storage.
Divide the lysate evenly between two 500 mL centrifuge tubes, centrifuge at 4000 g or higher at for 30 minutes.
Decant supernatant into 1 L 0.45 uM CA/PES filter, filter everything.
Take aliquots of the clarified lysate for qPCR/ddPCR titration, store aliquots at −20°−80° C.
   Titer these aliquots along with your final purified AAV later. This provides very helpful information in troubleshooting, allowing you to determine your purification efficiency, acting as a sanity check (i.e. the amount of purified AAV cannot be greater than the amount of AAV in input lysate, accounting for the accuracy of your qPCR/ddPCR titration) and in the case of low yields allows you to pinpoint whether the failure was at transfection or purification. HPLC purification and buffer exchange A full introduction into the usage and theory of HPLC is out of the scope of this protocol. The below is intended for users with basic training and capacity to operate HPLC machines and is based on the Akta Pure 25 system using pre-packed 1 mL AAVX columns. Regardless of the machine used, the below parameters are critical for efficient purification:
Purification is carried out at room temperature. Purification at cold temperatures substantially decreases binding efficiencies of the resin (FIG. 2D). For this purpose, both the clarified lysate containing AAV and the HPLC machine need to be brought to room temperature prior to purification.
   If the HPLC machine is housed in a fridge, we recommend not running the machine inside the fridge with cooling turned off and a closed door, since this can cause considerable increase of the temperature inside the fridge. Instead, we recommend either 1) placing the machine outside the fridge, 2) running the machine with fridge turned off and door left open or 3) hooking the fridge up to an external temperature controller (such as BN-LINK Digital Cooling Thermostat Controller, Amazon) and setting the set-point to 22° C. to 24° C.
AAVX resin binding capacity is not exceeded. Thermo Fisher indicates a binding capacity of up to 1×1014 vg/ml which varies between serotypes. A safe rule of thumb is to use 1 mL of resin per 1-2 hyperflasks, depending on the yield. When pooling AAV from multiple hyperflasks, we recommend packing your own columns with AAVX resin at a higher volume.
Lysate application speed does not exceed 1 mL/min for a 1 mL resin—i.e. the residence time is no less than 1 min. Resin dynamic binding capacity decreases with increasing loading speed and may result in more AAV in the flow-through. We recommend a 2 min residence time, or 0.5 mL/min loading speed for a 1 mL resin for maximum recovery.
Elution is performed in up-flow. Elution in down-flow (or the same flow direction as lysate application) results in approximately 20% less AAV in the elution. This is most likely because a majority of AAV binds close to the inlet on the resin; therefore eluting in the opposite direction avoids AAV rebinding of the resin at the elution step.
Elution pH is less than 2.9, with pH 2-2.5 optimal. AAV is acid stable at below pH 3 and will not elute above pH 2.9. Some serotypes can have strong binding affinities to the resin (such as AAV9 to the POROS AAV9 resin) and decreasing pH down to pH 2 can help increase recovery for such serotypes (Thermo Fisher—personal communication).
Resin regeneration is carried out with at least 15 min of 0.1M pH1 Phosphoric acid, followed by at least 15 min of 6M Guanidine. While not a concern with small-scale preps, at large scale preps (3×1013 . . . 2×1014) we commonly observed decreased binding efficiencies with resin re-use when 10 min of 6M
   Guanidine only was used, particularly for PHP.eB. The AAVX resin is highly acid stable, and increasing regeneration to 15 min of 0.1M pH1 Phosphoric acid, followed 15 min of 6M Guanidine restored binding efficiencies at large scales for at least 6 runs (Fig. S3).
The AAVX resin is NOT stable in high pH solutions. Accidental treatment of the resin with 0.1M NaOH will destroy the resin.

### HPLC Purification Protocol:

Bring the HPLC machine and the sample to room temperature
Prepare filtered HPLC buffers as described in the Required Items section. NB! 6M Guanidine and 0.1M Phosphoric acid are hazardous: use proper PPE and precautions in handling and disposal.
Connect the AAVX column to the machine using wet connection.
   (Manually flow some liquid out of the inlet, connect the column inlet connector in the wet environment to avoid introducing air into the column; repeat for column outlet).
Place buffer lines A1 to A6 and B1 into the corresponding buffers. Place Sample line (S1) and Sample Buffer line into 1×TBS.
   Tape the lines to the buffer bottles if they do not come with weights.
   Cover the bottles with parafilm to avoid evaporation and contamination
Prime inlets and purge pumps (see Akta Pure user manual section 5.4, pages 160-171)
   Prime the Sample inlet (S1) with TBS to avoid loss of your sample
Optional but recommended: place a 1 L bottle in the HPLC Outlet line to collect flow-through.
   This is useful if for whatever reason AAV fails to bind to the resin (such as fouling, low temp, or operator error), as it allows re-purification of the prep.
Pipette 0.11 mL of 1M Tris pH8+0.1% vol/vol Pluronic F68 neutralization buffer into 25 15 mL tubes; place them into the fraction collector, and set the fraction collector position to 1.
Place sample line (S1) into the AAV clarified lysate
   Place the bottle on the machine tilted and place the 51 line at the bottom most area, to be able to collect 100% of the lysate.
Ensure that there is sufficient volume of buffer for all buffers, and that all inlet lines are fully submerged.
Adjust the volume, speed or other parameters of the AAVX_HPLC_S1 Akta run protocol as necessary.
   Import the AAVX_HPLC_S1 protocol file on Akta Pure. Created with Unicorn v7.1.
   For other HPLC systems, see full overview of the run protocol below (HPLC run method)
Print out and go through the starting checklist every time before starting a new run (below).
Open the and start the AAVX_HPLC_S1 protocol.
   Depending on the volume of lysate and run speed, the run may take anywhere between 1 hour and 36 hours. We recommend doing the calculation before-hand to pick up elution fractions soon after the run is done.
   We recommend staying with the machine for the first 10-15 minutes in every new run to ensure that no leaks are present, that the sample inlet contains no air bubbles (which can cause pre-mature termination of loading, see Troubleshooting), and that sample application reaches a steady plateau.
   See Troubleshooting for examples of successful and unsuccessful runs
After the run is complete, remove the elution fractions containing AAV, store them at 4° C. for short term or −20° C. for long term.
Multiple preps can be automatically purified back-to-back by copying the protocol, changing sample input to S2 . . . S7, saving the new protocols, and starting them during the run of the first protocol. This adds them to the run cue, and the machine will automatically continue to these protocols after the previous ones are complete.
   Note that purification of multiple preps back-to-back removes the ability to collect flowthrough with the Outlet tube, as the machine unfortunately contains only a single outlet line.
   In this case, place the Outlet tube into waste bin to avoid overflowing the collection bottle.
After the last run, Remove the AAVX column, cap the tubes and store it at 4° C.
When the machine will not be expected to be used for longer than a week, perform System CIP (cleaning in place):
   Place all inlets used in the run into 1 L 0.1M NaOH
   Start the System CIP protocol or manually run 20 mL liquid through all lines
   Repeat for H₂O and 20% ethanol
   Store all lines in 20% ethanol

### Buffer Exchange

Before pipetting AAV containing solutions, we recommend coating all pipette tips and serologicals with Final Formulation Buffer (1×PBS, 35 mM NaCl and 0.001% Pluronic F68) or another Pluronic F68 containing buffer to minimize AAV binding to plastic. We also recommend usage of low-retention tips if available.
Thaw elution fractions if frozen previously.
From here on, work in sterile conditions.

### Protocol for Buffer Exchange Using Amicon Stirred Cell

While fractions are thawing, place 50 mL tubes on a rack in a TC hood and remove caps.
Attach 0.2 μm PES filter disks on top of the tubes by wrapping parafilm around the edge of the filter tightly.
Remove plungers from 50 mL syringes, and insert the syringes into the filter disks.
Pipette 2 mL of Final Formulation Buffer (1×PBS, 35 mM NaCl and 0.001% Pluronic F68) right onto the filter inlet, ensuring the buffer wets the filter.
Incubate for 15 min or more.
Assemble Amicon Stirred Cell manifold with the 100 kDa ultrafiltration disk at the bottom, glossy side facing upwards, inside the TC hood.
   See User guide here https://www.merckmillipore.com/FI/en/product/Amicon-Stirred-Cell-50mL.MM_NF-UFSC05001 and here: https://www.merckmillipore.com/FI/en/product/Ultrafiltration-Discs_100kDa-NMW,MM_NF-PLHK04310?bd=1#anchor_BRO
   Before first use, we recommend sterilizing the manifold and the filter disk. The filter disk can be sterilized in 70% ethanol for 5 minutes. For manifold sterilization, see Decontaminate and clean at the end of this section.
Place the Amicon on a magnetic stirrer and connect to the nitrogen tank.
   Tape the tube down if it does not stay in place, to keep the manifold on the magnetic stirrer.
Remove the top of the manifold and pour 50 mL of Final Formulation Buffer into the Amicon manifold.
Incubate for 15 min or more.
Open the nitrogen flow to pressurize the system.
   Slowly open the nitrogen tank first, then slowly open the course stage regulator on the right, finally slowly open the fine stage regulator to allow nitrogen flow into the manifold. Always follow manufacturer safety instructions.
   Be mindful to not exceed Amicon and filtration membrane maximum pressure limits—75 psi and 70 psi respectively.
   Turn on magnetic stirrer at low speed to check the functioning of the entire system.
   Turn off nitrogen flow from fine stage regulator when approximately 2-3 mL of liquid is left.
   NB! Turning off nitrogen flow does not immediately eliminate pressure from the manifold! To immediately eliminate pressure, slowly open the blue valve on the manifold cap.
Once elution fractions are thawed, centrifuge tubes containing elution fractions briefly to spin down the liquid.
Sterilize tubes with 70% ethanol and bring them to the tissue culture hood.
Pool all elution fractions containing AAV into a single 50 mL tube.
   Coat all pipette tips with sterile Final Formulation Buffer before aspirating AAV.
   Before aspirating liquid from a fraction, mix it by pipetting briefly to ensure there is no concentrated layer of AAV that remains at the bottom.
Bring the volume up to 48 mL with Final Formulation Buffer and mix.
Pour or pipette the volume into the 50 mL syringe filter, insert plunger and filter through the disk.
   Bringing the volume up to a total of 48 mL effectively dilutes the AAV, minimizing losses at the filtration step.
Pour or pipette the filtered solution into the previously assembled Amicon manifold.
Seal the Amicon cap, turn on the magnetic stirrer at low to medium speed, and open nitrogen flow to pressurize the system.
   Adjust the nitrogen pressure to provide continuous but not too rapid filtration. The filtration should take 5 minutes or more to completion. Rapid filtration results in high local densities of AAV on the filter surface, which leads to AAV aggregate formation and subsequent loss of titer and/or bioactivity. The stirring action by magnetic stir bar mitigates this substantially but would be reduced by very rapid filtration.
   Filtration speed is proportional to the concentration of AAV (and other molecules above 100 kDA). Thus, more concentrated preps will require longer time to filter—up to 15-20 minutes in our hands.
Stop the filtration once 2-5 mL of liquid is left.
   Turn off the nitrogen gas, then slowly and gently lift the blue valve to de-pressurize
   NB. Do not allow filtration to proceed to overconcentration at this step—this can cause AAV sedimentation and loss. Always aim to stop the filtration before 1 mL of liquid is left.
Remove the Amicon cap, pour in Final Formulation Buffer to 50 mL, repeat filtration
Repeat filtration until a total of >1000× dilution is achieved. This normally takes 2-3 filtration cycles.
   When leaving 5 mL of filtrate left, adding 45 mL of Final Formulation buffer results in 10× dilution. In this case 1000× dilution is achieved in three cycles.
   This can also be achieved in two steps if starting with <5 mL of volume of elution fractions (approximately 10× dilution at the filtration step) and repeating the buffer exchange twice with >10× dilution.
At the last filtration cycle, to obtain an accurate desired volume of final AAV, carefully observe the liquid level and stop and de-pressurize the system at 5 mL.
Stop the stirrer and estimate liquid volume by eye or by measuring with a serological pipette.
Remove the waste tube (keeping the tube connector attached to the manifold) and place a 5 mL tube underneath the waste outlet.
Continue filtration to the desired amount with stir bar turned on low, by estimating remaining volume in the Amicon manifold through observing the volume of waste in the 5 mL tube.
   This is required because the Amicon manifold is flat and accurate volume estimations at less than 2 mL are difficult. By estimating starting volume and volume in waste, an accurate estimation of volume left in the Amicon manifold can be made.
   Removal of the waste tube is necessary because it contributes to dead volume (approximately 2 mL), which can be filled with air to various degree, making accurate waste volume estimation difficult.
Alternatively, achieve desired final AAV volume by stopping the filtration at various points and measuring left-over volume with a pipette or serological.
The solution can accurately be concentrated to a few hundred microliters this way. However, we recommend keeping AAV at the highest possible volume allowed by downstream experimental requirements, particularly for PHP.B and its derivatives, as high concentrations of AAV will aggregate more readily during freeze-thaw cycles (Wright J F et al. Mol Ther. 2005 July; 12(1):171-8.).
Depressurize Amicon, remove stir bar and aspirate AAV into a new 1.5 mL or 2 mL tube.
   Optionally wash the filter membrane with additional 100 μL of Final Formulation Buffer and add to AAV.
Measure the volume of final AAV solution with a P1000 pipette or serological.
   For P1000, turn the pipette down to low volume, aspirate, then turn the pipette to higher volumes while the tip is submerged in the solution. When the solution is fully aspirated this way, the volume can be read from the pipette.
Record the volume or bring up to a desired volume with Final Formulation Buffer.
Take and store a 15 μl aliquot for titration
   Take a higher volume if further tests, such as protein electrophoresis are performed.
   This is to avoid unnecessarily thawing AAV designated for experimental applications.
   Do not open AAV tubes meant for experimental applications under non-sterile conditions.
Aliquot AAV into separate smaller aliquots unless the full amount is expected to be used in a single experiment.
Store AAV and the titration aliquot (if not immediately used for titration) at −80° C.
Decontaminate and clean the Amicon manifold between concentration of different AAV preps, and at the end:
   Disconnect the Amicon manifold from the nitrogen source
   Disassemble the manifold, and place all parts into a 2 L beaker filled with 500 mL of 5% hydrogen peroxide
   Incubate with slight shaking for 5 minutes
   Repeat with 70% ethanol and Final Formulation Buffer
   Dry before storage or concentration of a new prep
   Alternatively, based on manufacturer's datasheet, Amicon Stirred Cells are compatible with standard sterilizing gas mixtures or can be autoclaved for at least 10 cycles at 121° C., 1 bar (250° F., 15 psi) for 30 minutes.
      Protocol for buffer exchange using Amicon Ultra 15 Centrifuge devices
NB. Amicon Ultra 15 Centrifuge devices and their equivalents from competitors do NOT come sterilized. We recommend sterilization with sterilizing gases. If these cannot be used, 70% ethanol or UV can be attempted, but these may adversely affect the membrane (depending on the membrane type) and are likely of low efficacy.
While fractions are thawing, place the Amicon Ultra 15 tubes on a rack in a TC hood and remove caps.
Pipette 2 mL of Final Formulation buffer onto the membrane of the Amicon tubes.
Attach 0.2 μm PES filter disks on top of the tubes by wrapping parafilm around the edge of the filter tightly.
Remove plungers from 20 mL syringes and insert the syringes into the filter disks.
Pipette 2 mL of Final Formulation Buffer right onto the filter inlet, ensuring the buffer wets the filter.
Incubate for 15 min or more.
Once elution fractions are thawed, centrifuge tubes containing elution fractions briefly to spin down the liquid.
Sterilize tubes with 70% ethanol and bring them to the tissue culture hood.
Pool all elution fractions containing AAV into a single 15-50 mL tube.
   Coat all pipette tips with sterile Final Formulation Buffer before aspirating AAV.
   Before aspirating liquid from a fraction, mix it by pipetting briefly to ensure there is no concentrated layer of AAV that remains at the bottom.
Bring the volume up to 10 mL with Final Formulation Buffer and mix.
Pour or pipette the volume into the 20 mL syringe filter, insert plunger and filter through the disk.
   Bringing the volume up to max volume dilutes the AAV, minimizing losses at the filtration step.
Cap the Amicon tubes and centrifuge to concentrate:
   Continuous centrifugation will result in high local density of AAV at the filter membrane, causing aggregation and sedimentation out of the solution, which decreases titers and bioactivity.
   We recommend centrifuging for 1-2 minutes, removing the tubes from the centrifuge, opening them under the TC hood and mixing/washing the membrane with a P1000. This substantially reduces AAV aggregation although does not eliminate it for high concentration preps.
   NB. Aim to not concentrate below 1 mL, as this increases AAV aggregation.
   NB. Do not centrifuge at speeds higher than 5000 g!
Concentrate to 1 mL.
Decontaminate and bring the Amicon into a TC hood.
Fill the tube with Final Formation Buffer to 14 ml and mix.
Repeat until a total of >1000× dilution and desired final volume is achieved.
   The solution can be concentrated to a few hundred microliters. However, we recommend keeping AAV at the highest possible volume allowed by downstream experimental requirements, particularly for PHP.B and its derivatives, as high concentrations of AAV will aggregate more readily during buffer exchange as well as freeze-thaw cycles (Wright J F et al. Mol Ther. 2005 July; 12(1):171-8.).
Measure the volume of final AAV solution with a P1000 pipette or serological.
   For P1000, turn the pipette down to low volume, aspirate, then turn the pipette to higher volumes while the tip is submerged in the solution. When the solution is fully aspirated this way, the volume can be read from the pipette.
Record the volume or bring up to a desired volume with Final Formulation Buffer.
Take and store a 15 μl aliquot for titration.
   Take a higher volume if further tests, such as protein electrophoresis are performed.
   This is to avoid unnecessarily thawing AAV designated for experimental applications.
   Do not open AAV tubes meant for experimental applications under non-sterile conditions.
Aliquot AAV into separate smaller aliquots unless the full amount is expected to be used in a single experiment.
Freeze at −80 C.

### Titration

A thorough and detailed protocol for AAV titration using qPCR or ddPCR is described in Sanmiguel, J., Gao, G., & Vandenberghe, L. H. (2019). Quantitative and Digital Droplet-Based AAV Genome Titration. Adeno-Associated Virus Vectors, 51-83. doi:10.1007/978-1-4939-9139-6_4. We recommend performing AAV titrations based these protocols.

### Troubleshooting

A single hyperflask in our hands consistently yields an average of 3×1013 vg of purified AAV with well producing transgenes such as CMV-GFP for both single stranded vectors and self-complementary vectors. While some transgenes or serotypes may inherently produce at lower yields, yields below 1×1013 vg per hyperflask likely indicate a technical issue somewhere in the process.

Elution UV peak height roughly correlates with AAV yield. A peak of approximately the height (given efficient packing of the column) of the loading UV plateau for a single hyperflask generally indicates a yield of 1-3×1013 vg. Elution peaks much lower than (or with a lower area under the curve) indicate inefficient AAV production or purification. When no or very low elution peak is present, it is recommended to troubleshoot before proceeding to buffer exchange to save time and resources.

### EXAMPLES

### Troubleshooting

Low elution peak or low final AAV yield
Titer aliquots taken from the pre-purification lysate, flow-through and purified AAV to identify the step at which AAV was lost.
If pre-purification lysate contains low amounts of AAV, it is likely an AAV production issue.
If flow-through contains high amounts of AAV, it is likely an HPLC purification issue.
If pre-purification lysate contains high amounts of AAV and flow-through contains little AAV, it is likely a buffer exchange issue. Troubleshoot below accordingly.
   Low AAV production yield
Were HEK293T cells used?
Were cells of low passage?
Were cells not allowed to grow to confluency at any point during culture?
Were cells uniformly distributed during culture?
   Reasonably high elution peak—efficient production and purification. Left: chromatogram of the full run; right:
   magnified elution UV peak.
   Low elution peak—inefficient production and/or purification. Left: chromatogram of the full run; right:
   magnified elution UV peak.
Was transfection performed with cells at 70-80% confluency?
Was transfection performed with reagents at room temperature (not 4 degC)?
Was FBS excluded from the transfection mix?
Were all three plasmids (helper, cap and transgene) present at 260 μg:130 μg:130 μg ratios?
Was PEImax used?
Was PEImax used at the correct amount? (715 μg)
Were all three plasmids of the correct identity?
Did the transgene plasmid contain intact ITRs?
Mutated ITRs are one of the most common reasons for low yields. Mutated ITRs exist at some percent of the total population in most DNA preps. When the plasmid prep contains a high percent of mutated ITRs, yields are substantially reduced (up to 10-fold or more) and empty capsid percentage is increased. We recommend against use of such AAV preps to maintain experimental consistency and always checking for ITR integrity with SmaI/XmaI digests and/or next generation sequencing. For this reason, if it is known that a transgene will be extensively used in experimental studies, we recommend producing a Mega or Giga scale DNA prep, validating the integrity of the ITRs of this prep, aliquoting and using it for all subsequent AAV production runs.
Was the detergent and nuclease lysis performed correctly (containing all components, and not for longer than 2 hours)?
   High amounts of AAV in the flow-through and other HPLC purification issues
Was purification carried out at room temperature?
Was the sample brough to room temperature prior to purification?
Was the resin/column new?
Was the AAVX resin not allowed to dry out during storage?
If not, was the resin/column previously regenerated with >15 min pH1 Phosphoric acid and >15 min

### 6M Guanidine?

If so, has the resin been used more than 10 times?
While some data indicate that properly regenerated resins can be used for up to at least 20 times, we recommend switching to a new resin if flow-through issues emerge after roughly uses.
Did the resin get exposed to 0.1M NaOH or other strong alkaline agents?
Is this a serotype validated to bind to AAVX or a new untested serotype?
Clogged column:
Was the lysate clarified with centrifugation and filtration before loading onto the HPLC?
Was upflow selected during elution and column regeneration?
Premature termination of sample loading:
Was the HPLC protocol set to “Inject all sample using air sensor” in the Sample

### Application Step

(on by default in the AAVX_HPLC_S1 protocol)?
If so, the system likely detected an air bubble in the sample line and proceeded to the following steps. We recommend purging the sample line as described in the Akta manual and repeating the purification.
Alternatively, Sample Application can be set to “Inject Fixed Sample Volume”—in this case sample volume must be accurately measured before to prevent underloading or loading air onto the column.
Continuously increasing preC pressure/sample pump pressure during loading:
The column is being clogged. If the lysate was clarified with centrifugation and filtration before loading onto the HPLC and resin regenerated correctly, this could be a frit issue, if self-packed columns are used. We recommend replacing frits. If pre-packed column was used, it is a manufacturer issue or a column reaching the end of its lifespan. Either way, we recommend using a new column.
Fluctuating UV line/preC line during loading:
There is likely an air bubble in the sample line. While purification can still work, it runs the risk of premature terminating the loading if “Inject all sample using air sensor” in the Sample

Application step is selected. We recommend terminating the program, placing the Sample line into a separate tube of TBS, manually performing priming and purging, placing the line back into sample and re-starting the run.

### Low Final AAV Yield/Buffer Exchange Issues

Was upflow used during HPLC elution?
Downflow decreases yields by approximately 20%.
Was Pluronic F68 used in the elution buffer?
Were elution fractions neutralized with pH8 Tris-0.01% Pluronic F68?
Were filters, plasticware and pipette tips coated with Final Formulation Buffer (or other Pluronic F68 containing buffer) during handling?
Was buffer exchange carried out according to instructions, preventing overconcentration?
AAV sedimentation (white cloudy particle formation):
Was buffer exchange carried out according to instructions, preventing overconcentration?
Some reports suggest some serotypes can be re-solubilized by gentle overnight shaking at room temperature. While most AAV serotypes are stable at room temperature for that duration, it is up to the user to decide whether this is worth trying.
Broken Amicon Ultra 15 tubes during centrifugation:
Was centrifugation speed kept below 5000 g?
Low filtration speed at buffer exchange:
This is likely a high concentration prep, or contains additional molecules that are retained by the buffer exchange membrane. We recommend patiently continuing purification as reasonable or high yields can still be obtained. Alternatively, the sample can be diluted and split between more buffer exchange units if available.
High filtration speed at buffer exchange:
The prep likely contains little to no AAV. Pure PBS filters fully in seconds. Decrease pressure or centrifugation speed.
Liquid leakage under the cap of Amicon Ultra 15 units during centrifugation:
Cap overtightened or more than 14 mL of media loaded onto the Amicon.
This is a particular problem with fixed-angle rotors. We recommend using a swinging bucket rotor if available.
Low buffer exchange efficiencies despite no clear technical flaw:
Take aliquots of all purification and buffer exchange steps to determine the exact step where loss occurs.
For high concentration preps, splitting the prep between multiple filtration/buffer exchange units can reduce loss.

### Starting Checklist

Sample filtered?
Aliquot of sample (cleared lysate) taken?
Column attached?
Correct orientation?
Correct resin?
All inlets in correct buffers?
A1: TBS
A2: 20% EtOH-TBS
A3: 2×TBS
A4: 6M Guanidine
A5: 20% EtOH
A6: Phosphoric acid 0.1M, pH1
B1: 0.2M Glycine-0.01% Pluronic F68
S1: Sample 1
S2: Sample 2 . . .
Buffer: TBS
Outlet in outlet tube
Enough buffer in each tube?
Outlet in outlet?
All inlets primed?
All pump heads purged?
Purge confirmed?
Sample pump purged?
Purge confirmed?
Fractionation:
Fraction collector set to position 0?
Enough tubes added for fractions? Apprx 20 tubes per run
Tris-Pluronic added to collection tubes?
Method
Correct method selected?
Correct outlets selected?
Correct sample application volume selected?
Correct fraction volumes selected?
Correct location for save file?
Enough volume in the sample to match method?
Waste empty?
Everything double-checked?

### HPLC Run Method

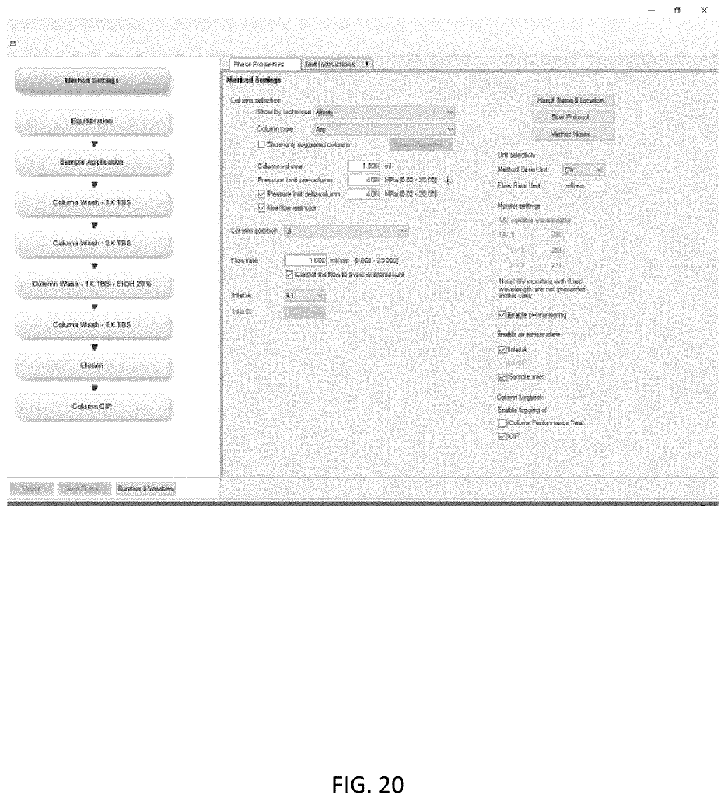

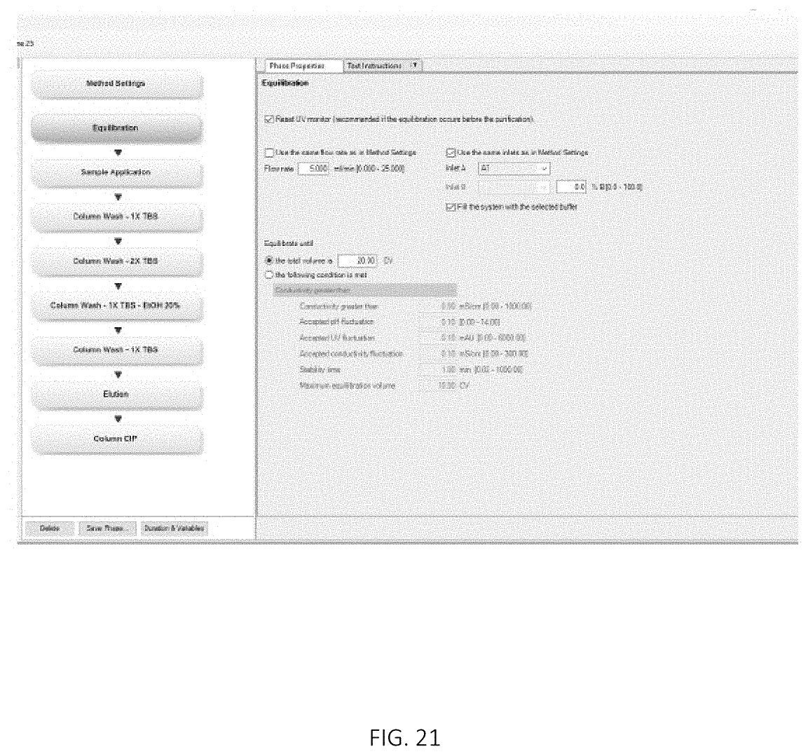

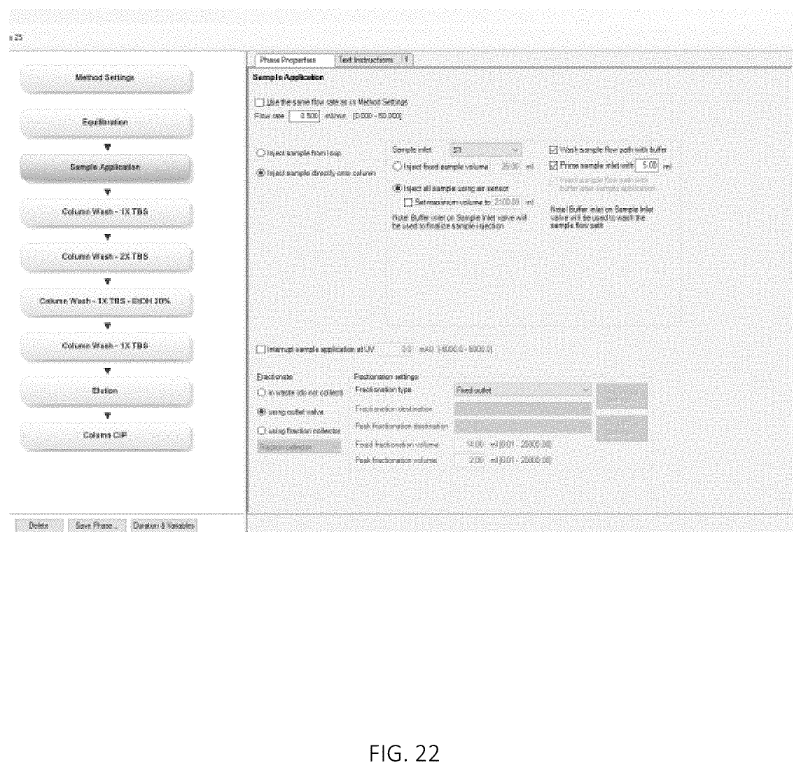

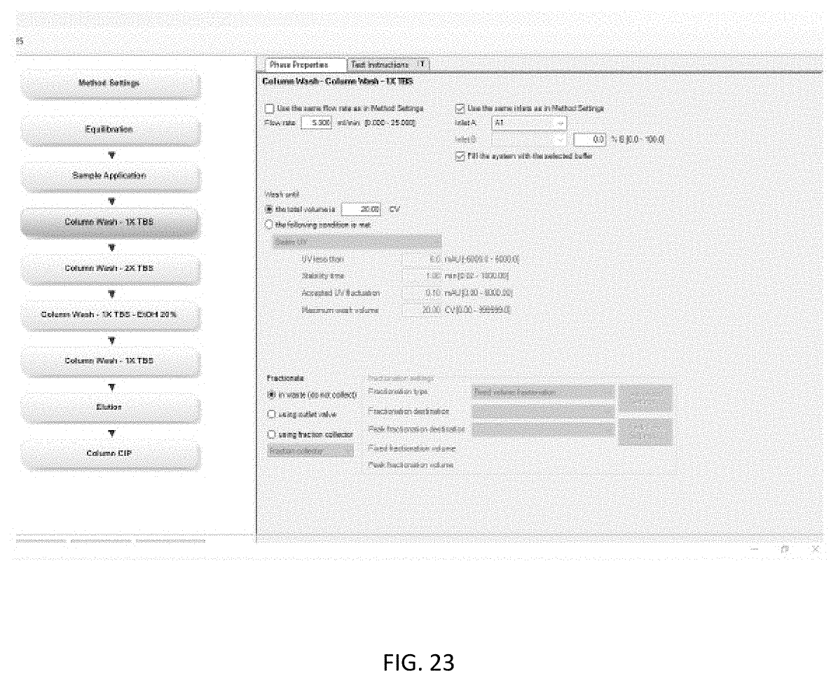

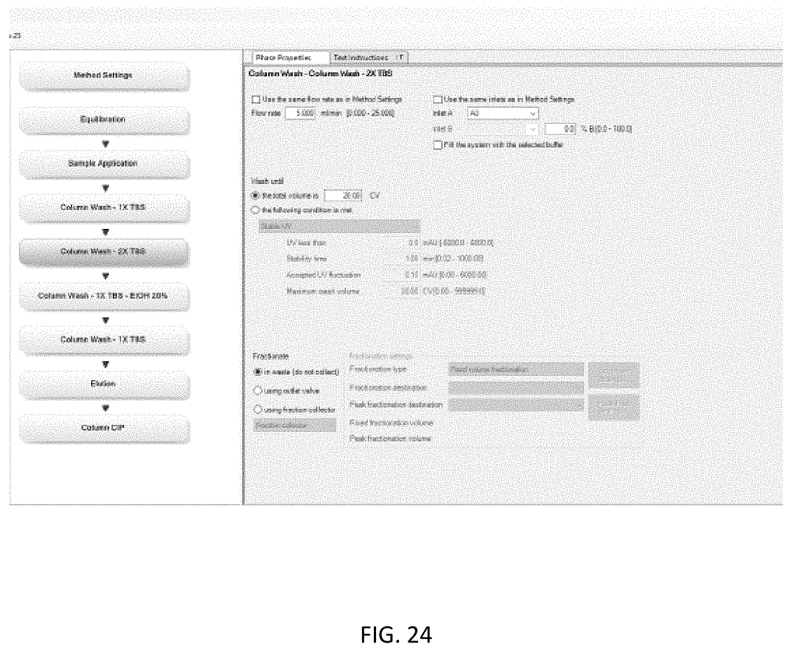

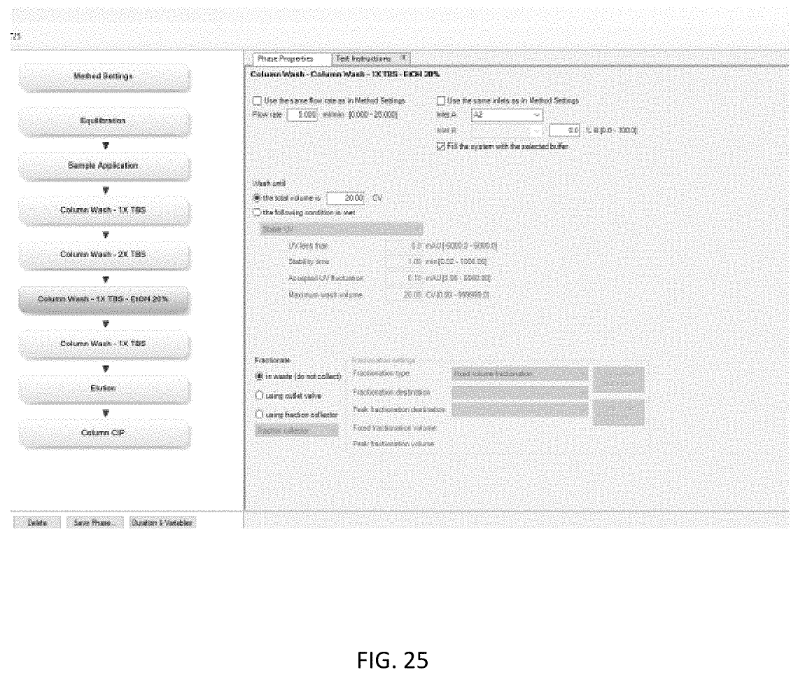

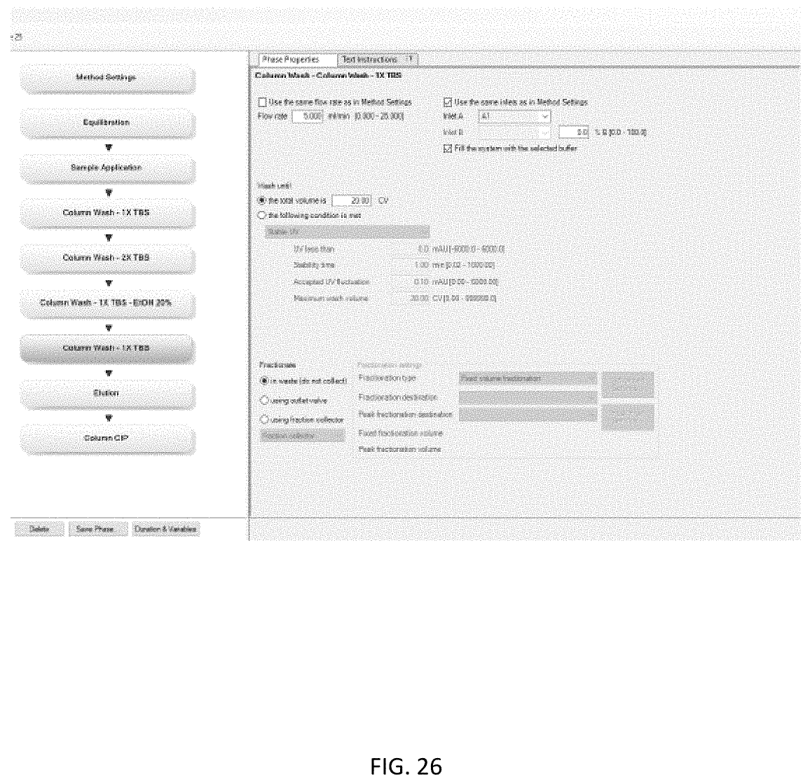

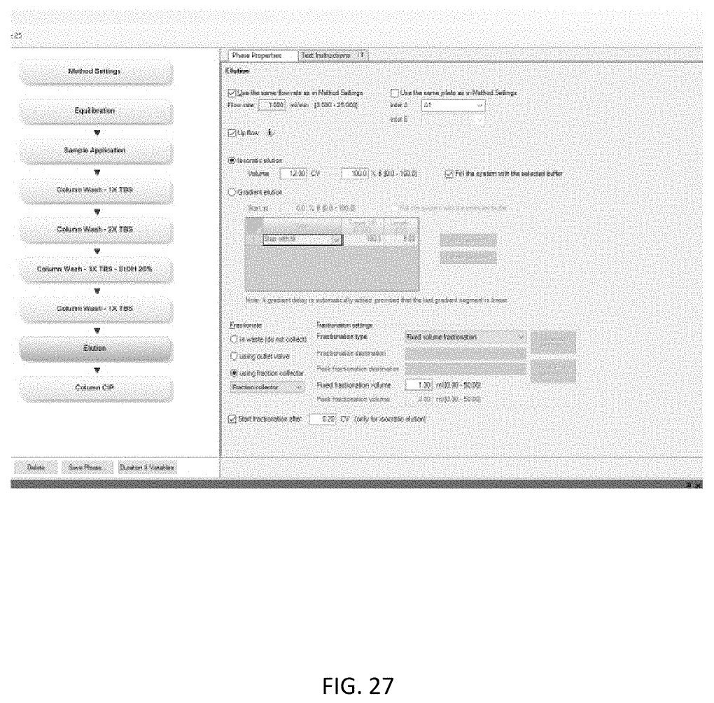

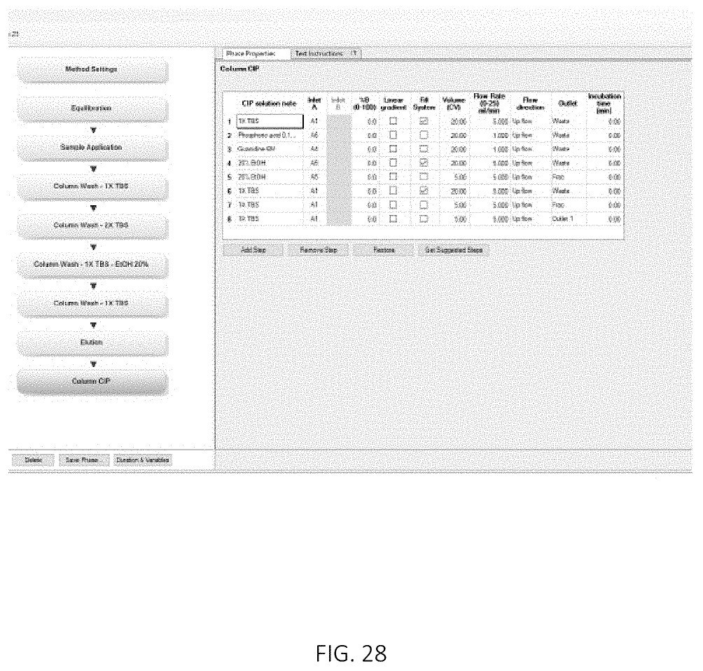

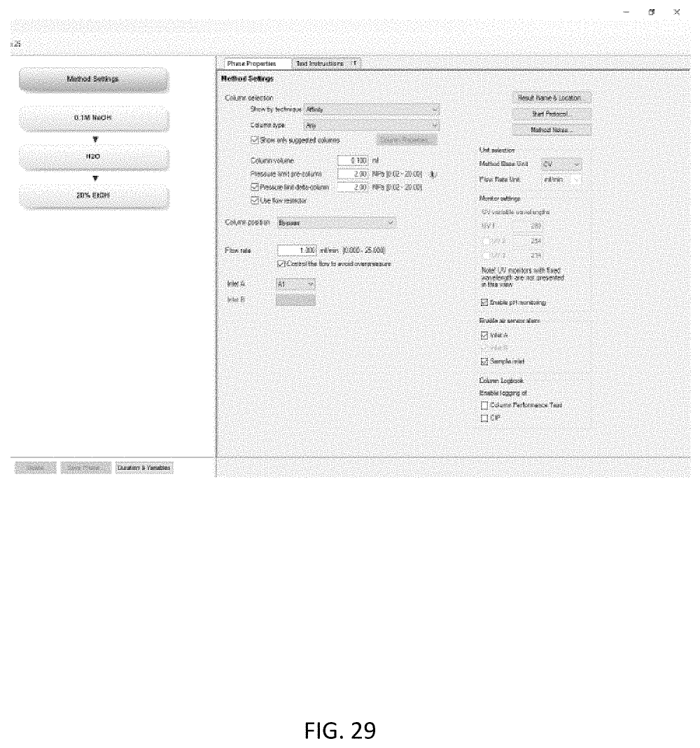

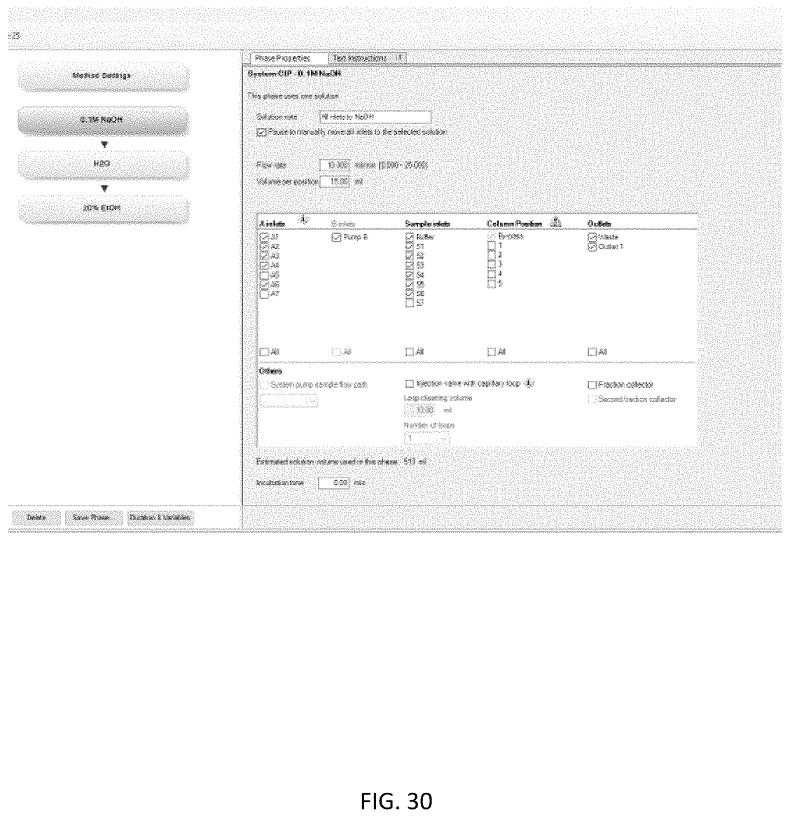

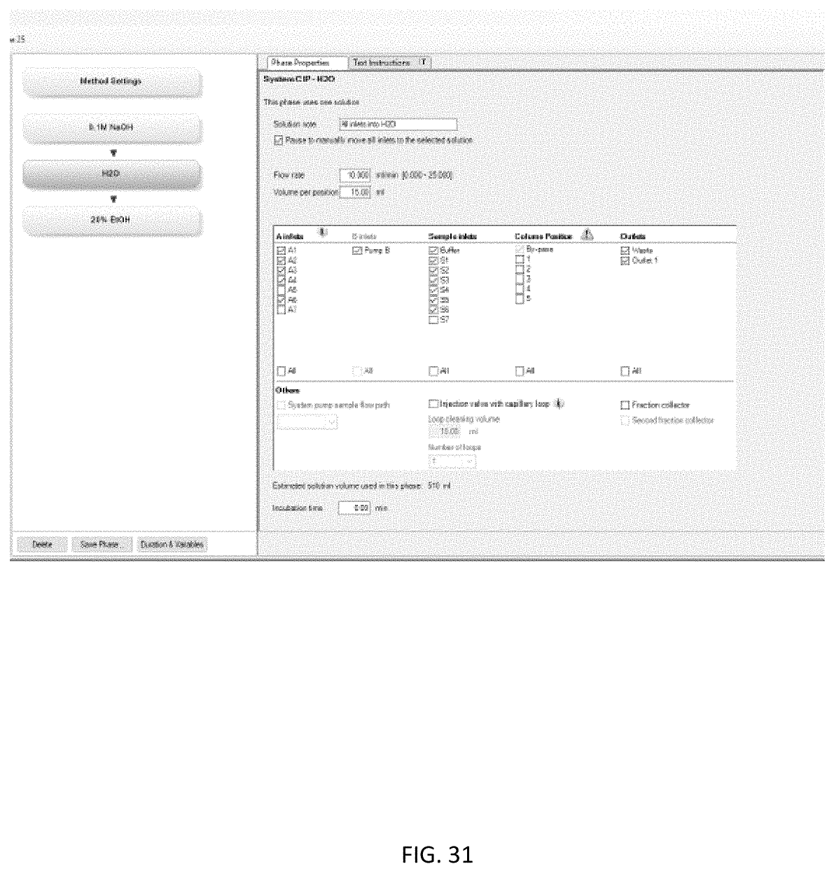

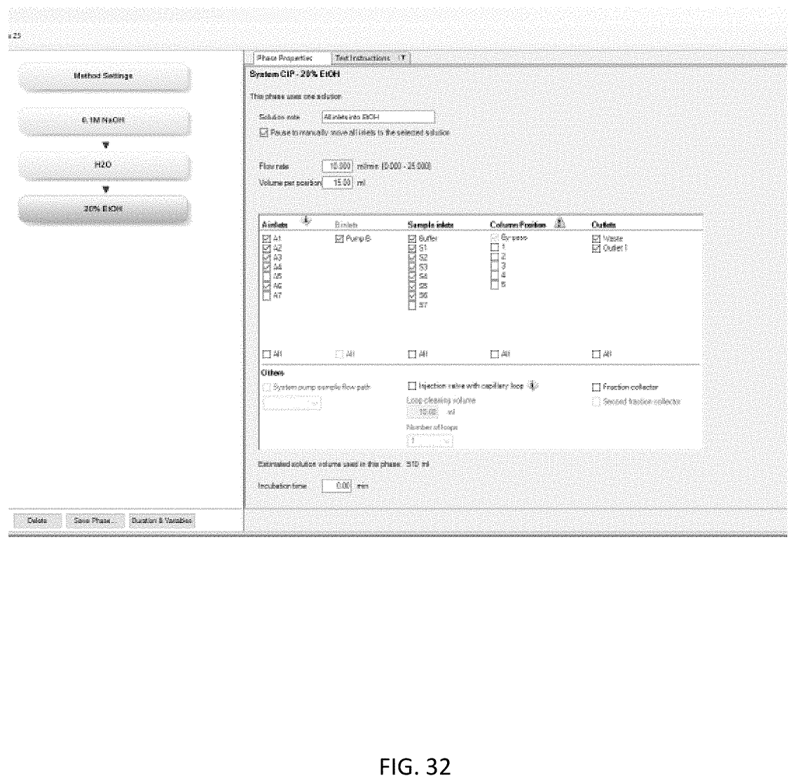

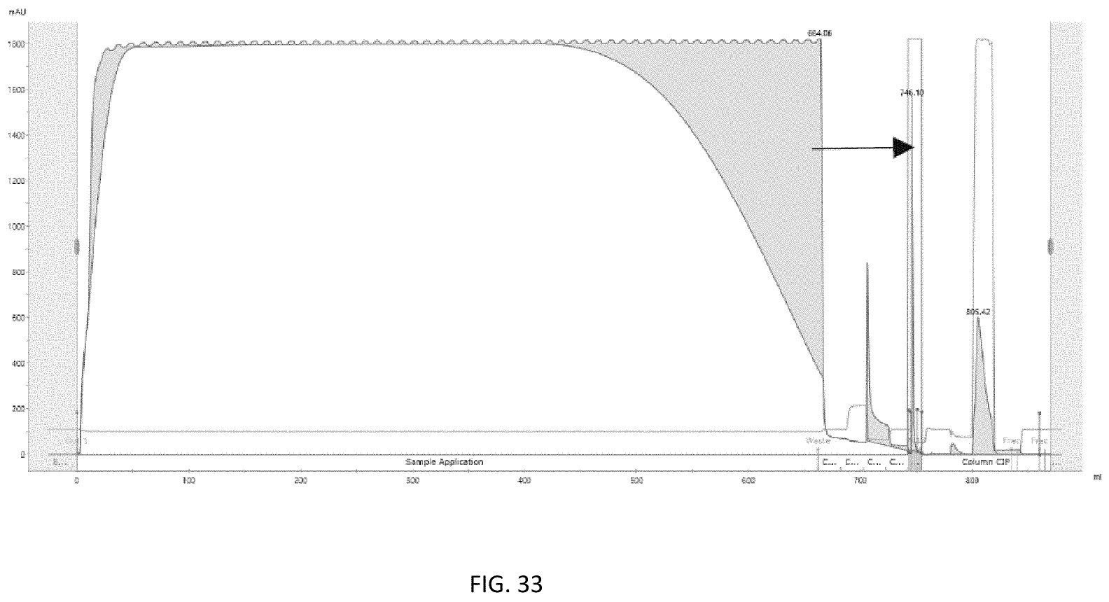

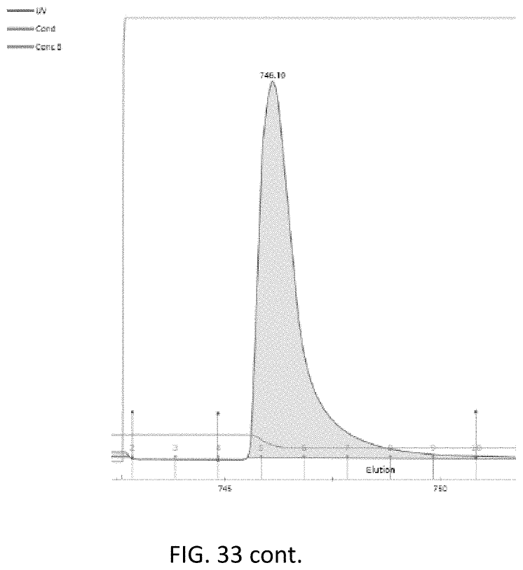

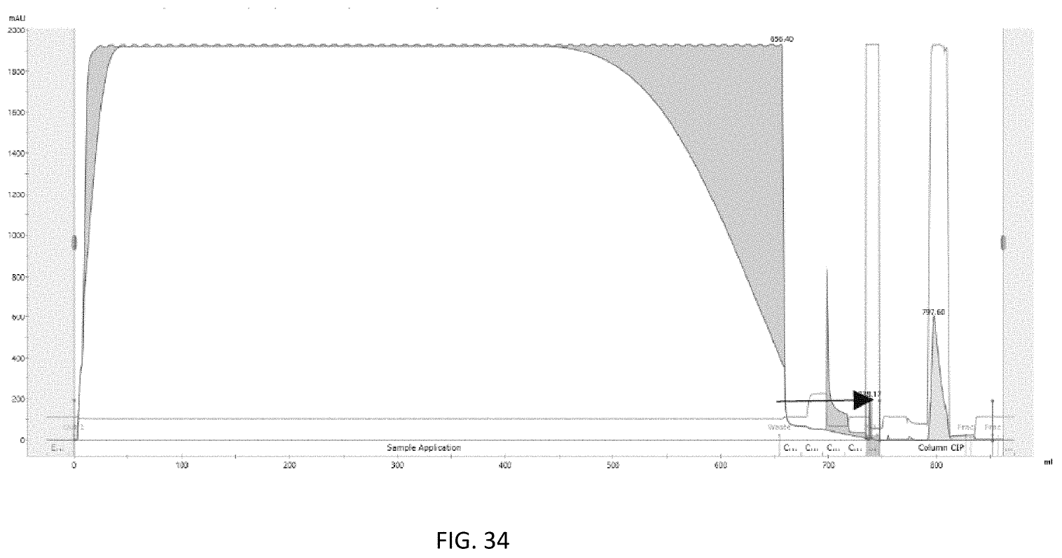

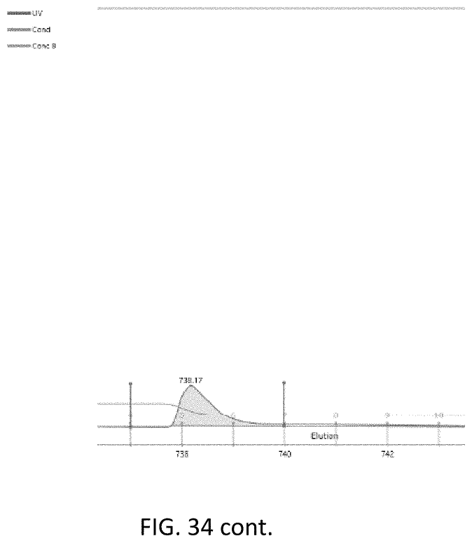

For users with Akta Pure systems we highly recommend importing the AAVX_HPLC_S1 and System_CIP protocols to avoid unwanted errors. FIGS. 20-32 is intended as a complete specification of run parameters for users of other HPLC systems.

### REFERENCES

1. Atchison, R. W., Casto, B. C., and Hammon, W. M. (1965). Adenovirus-Associated Defective Virus Particles. Science 149, 754-755.
2. Hoggan, M. D., Blacklow, N. R., and Rowe, W. P. (1966). Studies of small DNA viruses found in various adenovirus preparations: physical, biological, and immunological characteristics. Proceedings of the National Academy of Sciences 55, 1467-1474.
3. Samulski, R. J., and Muzyczka, N. (2014). AAV-Mediated Gene Therapy for Research and Therapeutic Purposes. Annual Review of Virology 1, 427-451.
4. Ginn, S. L., Amaya, A. K., Alexander, I. E., Edelstein, M., and Abedi, M. R. (2018). Gene therapy clinical trials worldwide to 2017: An update. The Journal of Gene Medicine 20, e3015.
5. Crosson, S. M., Dib, P., Smith, J. K., and Zolotukhin, S. (2018). Helper-free Production of Laboratory Grade AAV and Purification by Iodixanol Density Gradient Centrifugation. Molecular Therapy—Methods and Clinical Development 10, 1-7.
6. Lock, M., Alvira, M., Vandenberghe, L. H., Samanta, A., Toelen, J., Debyser, Z., and Wilson, J. M. (2010). Rapid, simple, and versatile manufacturing of recombinant adeno-associated viral vectors at scale. Human Gene Therapy 21, 1259-1271.
7. Clément, N., and Grieger, J. C. (2016). Manufacturing of recombinant adeno-associated viral vectors for clinical trials. Molecular Therapy—Methods and Clinical Development 3, 16002.
8. Ła̧cki, K. M., and Riske, F. J. (2020). Affinity Chromatography: An Enabling Technology for Large-Scale Bioprocessing. Biotechnology Journal 15, 1800397.
9. Rieser, R., Koch, J., Faccioli, G., Richter, K., Menzen, T., Biel, M., Winter, G., and Michalakis, S. (2021). Comparison of Different Liquid Chromatography-Based Purification Strategies for Adeno-Associated Virus Vectors. Pharmaceutics 13, 748.
10. Terova, O., Hermans, P., de Rooij, J., and Detmers, F. (2018). Overcoming Downstream Purification Challenges for Viral Vector Manufacturing: Enabling Advancement of Gene Therapies in the Clinic. Cell Gene Therapy Insights 4, 101-111.
11. Potter, M., Lins, B., Mietzsch, M., Heilbronn, R., Van Vliet, K., Chipman, P., Agbandje-McKenna, M., Cleaver, B. D., Clement, N., Byrne, B. J., Zolotukhin, S., et al. (2014). A simplified purification protocol for recombinant adeno-associated virus vectors. Molecular therapy. Methods & clinical development 1, 14034.
12. Nass, S. A., Mattingly, M. A., Woodcock, D. A., Burnham, B. L., Ardinger, J. A., Osmond, S. E., Frederick, A. M., Scaria, A., Cheng, S. H., and O'Riordan, C. R. (2018). Universal Method for the Purification of Recombinant AAV Vectors of Differing Serotypes. Molecular Therapy—Methods and Clinical Development 9, 33-46.
13. Wang, Q., Lock, M., Prongay, A. J., Alvira, M. R., Petkov, B., and Wilson, J. M. (2015). Identification of an adeno-associated virus binding epitope for AVB sepharose affinity resin. Molecular Therapy—Methods and Clinical Development 2, 15040.
14. Deverman, B. E., Pravdo, P. L., Simpson, B. P., Kumar, S. R., Chan, K. Y., Banerjee, A., Wu, W.-L., Yang, B., Huber, N., Pasca, S. P., and Gradinaru, V. (2016). Cre-dependent selection yields AAV variants for widespread gene transfer to the adult brain. Nature Biotechnology 34, 204-209.
15. Limberis, M. P., Vandenberghe, L. H., Zhang, L., Pickles, R. J., and Wilson, J. M. (2009). Transduction efficiencies of novel AAV vectors in mouse airway epithelium in vivo and human ciliated airway epithelium in vitro. Molecular Therapy 17, 294-301.
16. Zinn, E., Pacouret, S., Khaychuk, V., Turunen, Heikki T T, Carvalho, Livia S. S., Andres-Mateos, E., Shah, S., Shelke, R., Maurer, Anna C. C., Plovie, E., Xiao, R., et al. (2015). In silico reconstruction of the viral evolutionary lineage yields a potent gene therapy vector. Cell Reports 12, 1056-1068.
17. Limberis, M. P., Vandenberghe, L. H., Zhang, L., Pickles, R. J., and Wilson, J. M. (2009). Transduction efficiencies of novel AAV vectors in mouse airway epithelium in vivo and human ciliated airway epithelium in vitro. Mol Ther 17, 294-301.
18. Gao, G. P., Alvira, M. R., Wang, L., Calcedo, R., Johnston, J., and Wilson, J. M. (2002). Novel adeno-associated viruses from rhesus monkeys as vectors for human gene therapy. Proceedings of the National Academy of Sciences 99, 11854-11859.
19. Lin, J., Calcedo, R., Vandenberghe Luk, H., Bell, P., Somanathan, S., and Wilson James, M. (2009). A New Genetic Vaccine Platform Based on an Adeno-Associated Virus Isolated from a Rhesus Macaque. Journal of Virology 83, 12738-12750.
20. Dalkara, D., Byrne, L. C., Klimczak, R. R., Visel, M., Yin, L., Merigan, W. H., Flannery, J. G., and Schaffer, D. V. (2013). In Vivo Directed Evolution of a New Adeno-Associated Virus for Therapeutic Outer Retinal Gene Delivery from the Vitreous. Science Translational Medicine 5, 189ra176-189ra176.
21. Chiorini, J. A., Kim, F., Yang, L., and Kotin, R. M. (1999). Cloning and characterization of adeno-associated virus type 5. Journal of virology 73, 1309-1319.
22. Chiorini, J. A., Yang, L., Liu, Y., Safer, B., and Kotin, R. M. (1997). Cloning of adeno-associated virus type 4 (AAV4) and generation of recombinant AAV4 particles. Journal of Virology 71, 6823-6833.
23. Vandenberghe, L. H., Wang, L., Somanathan, S., Zhi, Y., Figueredo, J., Calcedo, R., Sanmiguel, J., Desai, R. A., Chen, C. S., Johnston, J., Grant, R. L., et al. (2006). Heparin binding directs activation of T cells against adeno-associated virus serotype 2 capsid. Nature Medicine 12, 967-971.
24. J, B., J F, W., A, K., J B, J., B, H., O, Z., F, M., D, H., D, C., T S, R., Z, W., et al. (2008). Reversal of blindness in animal models of leber congenital amaurosis using optimized AAV2-mediated gene transfer. Molecular Therapy 16, 458-465.
25. Patrick), M. I., Cox, C. I., Blue, C., Barnard, A. R., Martinez-Fernandez de la Camara, C., and MacLaren, R. E. (2020). Inclusion of PF68 Surfactant Improves Stability of rAAV Titer when Passed through a Surgical Device Used in Retinal Gene Therapy. Molecular Therapy—Methods and Clinical Development 17, 99-106.
26. Dias Florencio, G., Precigout, G., Beley, C., Buclez, P. O., Garcia, L., and Benchaouir, R. (2015). Simple downstream process based on detergent treatment improves yield and in vivo transduction efficacy of adeno-associated virus vectors. Molecular Therapy—Methods and Clinical Development 2, 15024.
27. Strobel, B., Miller, F. D., Rist, W., and Lamla, T. (2015). Comparative Analysis of Cesium Chloride- and Iodixanol-Based Purification of Recombinant Adeno-Associated Viral Vectors for Preclinical Applications. Human Gene Therapy Methods 26, 147-157.
28. Burova, E., and loffe, E. (2005). Chromatographic purification of recombinant adenoviral and adeno-associated viral vectors: methods and implications. Gene Therapy 12, S5-S17.
29. Savy, A., Dickx, Y., Nauwynck, L., Bonnin, D., Merten, O. W., and Galibert, L. (2017). Impact of Inverted Terminal Repeat Integrity on rAAV8 Production Using the Baculovirus/Sf9 Cells System. Human Gene Therapy Methods 28, 277-289.
30. Wang, C., Mulagapati, S. H. R., Chen, Z., Du, J., Zhao, X., Xi, G., Chen, L., Linke, T., Gao, C., Schmelzer, A. E., and Liu, D. (2019). Developing an Anion Exchange Chromatography Assay for Determining Empty and Full Capsid Contents in AAV6.2. Molecular Therapy—Methods & Clinical Development 15, 257-263.
31. Khatwani, S. L., Pavlova, A., and Pirot, Z. (2021). Anion-exchange HPLC assay for separation and quantification of empty and full capsids in multiple adeno-associated virus serotypes. Molecular Therapy—Methods & Clinical Development 21, 548-558.
32. Joshi, P. R. H., Bernier, A., Moço, P. D., Schrag, J., Chahal, P. S., and Kamen, A. (2021). Development of a scalable and robust AEX method for enriched rAAV preparations in genome-containing VCs of serotypes 5, 6, 8, and 9. Molecular Therapy—Methods & Clinical Development 21, 341-356.
33. Urabe, M., Xin, K. Q., Obara, Y., Nakakura, T., Mizukami, H., Kume, A., Okuda, K., and Ozawa, K. (2006). Removal of empty capsids from type 1 adeno-associated virus vector stocks by anion-exchange chromatography potentiates transgene expression. Molecular Therapy 13, 823-828.
34. Qu, G., Bahr-Davidson, J., Prado, J., Tai, A., Cataniag, F., McDonnell, J., Zhou, J., Hauck, B., Luna, J., Sommer, J. M., Smith, P., et al. (2007). Separation of adeno-associated virus type 2 empty particles from genome containing vectors by anion-exchange column chromatography. Journal of Virology Methods 140, 183-192.
35. Wang, C., Mulagapati, S. H. R., Chen, Z., Du, J., Zhao, X., Xi, G., Chen, L., Linke, T., Gao, C., Schmelzer, A. E., and Liu, D. (2019). Developing an Anion Exchange Chromatography Assay for Determining Empty and Full Capsid Contents in AAV6.2. Molecular Therapy—Methods and Clinical Development 15, 257-263.
36. McIntosh, N. L., Berguig, G. Y., Karim, O. A., Cortesio, C. L., De Angelis, R., Khan, A. A., Gold, D., Maga, J. A., and Bhat, V. S. (2021). Comprehensive characterization and quantification of adeno associated vectors by size exclusion chromatography and multi angle light scattering. Scientific Reports 11, 3012.
37. Gagnon, P., Leskovec, M., Prebil, S. D., Zigon, R., Štokelj, M., Raspor, A., Peljhan, S., and Štrancar, A. (2021). Removal of empty capsids from adeno-associated virus preparations by multimodal metal affinity chromatography. Journal of Chromatography A 1649, 462210.
38. Gimpel, A. L., Katsikis, G., Sha, S., Maloney, A. J., Hong, M. S., Nguyen, T. N. T., Wolfrum, J., Springs, S. L., Sinskey, A. J., Manalis, S. R., Barone, P. W., et al. (2021). Analytical methods for process and product characterization of recombinant adeno-associated virus-based gene therapies. Molecular Therapy—Methods and Clinical Development 20, 740-754.
39. Fu, X., Chen, W. C., Argento, C., Clarner, P., Bhatt, V., Dickerson, R., Bou-Assaf, G., Bakhshayeshi, M., Lu, X., Bergelson, S., and Pieracci, J. (2019). Analytical Strategies for Quantification of Adeno-Associated Virus Empty Capsids to Support Process Development. Human Gene Therapy Methods 30, 144-152.
40. Sanmiguel, J., Gao, G., and Vandenberghe, L. H. (2019). Quantitative and digital droplet-based AAV genome titration. Methods in Molecular Biology 1950, 51-83.
41. Schindelin, J., Arganda-Carreras, I., Frise, E., Kaynig, V., Longair, M., Pietzsch, T., Preibisch, S., Rueden, C., Saalfeld, S., Schmid, B., Tinevez, J. Y., et al. (2012). Fiji: An open-source platform for biological-image analysis. Nature Methods 9, 676-682.
42. Sievers, F., Wilm, A., Dineen, D., Gibson, T. J., Karplus, K., Li, W., Lopez, R., McWilliam, H., Remmert, M., Soding, J., Thompson, J. D., et al. (2011). Fast, scalable generation of high-quality protein multiple sequence alignments using Clustal Omega. Mol Syst Biol 7, 539.
43. Goujon, M., McWilliam, H., Li, W., Valentin, F., Squizzato, S., Paern, J., and Lopez, R. (2010). A new bioinformatics analysis tools framework at EMBL-EBI. Nucleic Acids Res 38, W695-699.
44. Darriba, D., Taboada, G. L., Doallo, R., and Posada, D. (2011). ProtTest 3: fast selection of best-fit models of protein evolution. Bioinformatics 27, 1164-1165.
45. Le, S. Q., and Gascuel, 0. (2008). An improved general amino acid replacement matrix. Mol Biol Evol 25, 1307-1320.
46. Kumar, S., Stecher, G., Li, M., Knyaz, C., and Tamura, K. (2018). MEGA X: Molecular Evolutionary Genetics Analysis across Computing Platforms. Mol Biol Evol 35, 1547-1549.
47. Revell, L. J. (2012). phytools: An R package for phylogenetic comparative biology (and other things. Methods in Ecology and Evolution 3, 217-223.

1. A method of high efficiency purification of adeno-associated virus (AAV) particles comprising the steps of:
a. providing a cell lysate that comprises one or more AAV particles;
b. contacting the cell lysate comprising the one or more AAV particles with a volume of chromatography resin medium comprising at least one ligand possessing a pan-AAV affinity, wherein said contacting induces binding of the one or more AAV particles to the chromatography resin medium and wherein the contacting is performed at a temperature of between approximately 20° C. to approximately 28° C. and/or both the cell lysate and the chromatography resin medium are allowed to come to a temperature between approximately 20° C. to approximately 28° C. previous to said contacting; and
c. eluting the bound one or more AAV particles from the chromatography resin medium using a volume of an acidic elution buffer solution to provide one or more eluted volume fractions containing the one or more AAV particles;
whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, and wherein the method does not require modifications contingent on the AAV serotype being purified to obtain said efficiency.

2. The method according to claim 1, wherein the contacting is performed at a temperature of between approximately 22° C. to 26° C., or
wherein both the cell lysate and the chromatography resin medium are allowed to come to a temperature between approximately 22° C. to approximately 26° C. prior to the contacting step.

3.-7. (canceled)

8. The method according to claim 1, wherein the one or more AAV particles subjected to purification is selected from one or more AAV serotypes from the group consisting of AAV1, AAV2, AAV2-7m8, AAV-HSPG, AAV3, AAV4, AAV5, AAV6, AAV6.2, AAV7, AAV8, AAV9, Rh10, Rh61, Rh8, Rh32.33, VR-865, PHP.B and Anc80.

9. The method according to claim 1, wherein the volume of acidic elution buffer solution comprises glycine, phosphoric acid or citric acid and/or wherein the pH of the acidic elution buffer solution is approximately 3.0 or less.

10. (canceled)

11. (canceled)

12. (canceled)

13. The method according to claim 1, wherein the chromatography resin medium comprising a ligand possessing a pan-AAV affinity comprises a cross-linked poly(styrene-divinylbenzene) bead coated with a cross-linked polyhydroxylated polymer.

14. The method according to claim 1, wherein the chromatography resin medium is linked to a ligand which comprises a camelid heavy-chain-only single domain antibody fragment and/or wherein the chromatography resin medium comprises one or more beads with a diameter of approximately 50 μm.

15. (canceled)

16. (canceled)

17. The method according to claim 1, wherein the one or more AAV particles comprise a capsid which encapsulates vector DNA or which is empty.

18. The method according to claim 17, wherein the method further comprises one or more steps selected from a downstream step of performing size exclusion or anion exchange chromatography on the purified one or more AAV particles thereby enriching the AAV particles which encapsulate said vector DNA and performing one or more upstream steps to reduce AAV particles comprising an empty capsid including optimizing plasmid transfection ratios, utilizing vector plasmids that are full length or have minimal ITR deletion, using novel engineered ITRs, and using a transfection plasmid containing both the AAV cap and transgene in cis.

19. The method according to claim 1, wherein one or more steps of the method are carried out in the presence of a non-ionic surfactant, wherein the non-ionic surfactant is a tri-block poly(ethylene oxide) (PEO)-poly(propylene oxide) (PPO)-poly(ethylene oxide) (PEO) copolymer.

20. (canceled)

21. (canceled)

22. The method according to claim 1, further comprising a step of neutralizing the one or more eluted fractions containing the one or more AAV particles, wherein the step of neutralizing comprises addition of tris(hydroxymethyl)aminomethane hydrochloride (TRIS-HCL) to the one or more elution fractions.

23. (canceled)

24. The method according to claim 1, wherein the method further comprises one or more washing steps prior to, after, or both prior to and after the elution step, wherein the one or more washing steps are carried out with a washing buffer solution comprising one or more components selected from the group consisting of tris-buffered saline (TBS), ethanol, guanidine HCL, phosphoric acid, glycine, Tris NaOH, water, a non-ionic surfactant, and NaCl.

25. (canceled)

26. The method according to claim 1, further comprising a step of regenerating the chromatography resin medium comprising the at least one ligand possessing a pan-AAV affinity by contacting the medium with at least one acidic component, wherein contact with the at least one acidic component is carried out for a predetermined amount of time, wherein the predetermined amount of time is 30 seconds to 24 hours, 1 minute to 12 hours, 5 minutes to 4 hours, 10 minutes to 1 hour, or 15 minutes to 30 minutes.

27. The method according to claim 26, wherein the at least one acidic component comprises phosphoric acid and/or guanidine HCL, or
wherein the at least one acidic component comprises two acidic components which are each contacted with the chromatography resin concurrently or sequentially, and wherein the contact with each of the two acidic components is carried out for predetermined amounts of time.

28. (canceled)

29. (canceled)

30. (canceled)

31. The method according to claim 26, wherein the regeneration step is carried out by contacting the chromatography resin medium with 0.1M phosphoric acid for at least 15 minutes, followed or preceded by contacting the chromatography resin medium with 6M guanidine for at least 15 minutes.

32. (canceled)

33. (canceled)

34. (canceled)

35. The method according to claim 1, wherein the chromatography resin medium linked to at least one ligand which possess pan-AAV affinity is packed into a column to provide a high performance liquid chromatography column and/or
wherein the volume of chromatography resin medium is at least 0.1 mL, at least 0.5 mL, at least 1 mL, at least 2 mL, at least 3 mL, at least 4 mL, at least 5 mL, or at least 10 mL.

36. (canceled)

37. The method according to claim 1, wherein a flow rate of one or both of the acidic elution buffer solution and the wash buffer solution through the chromatography resin medium during the elution or washing steps comprises a flow rate of no more than 5 mL/min, no more than 4 mL/min, no more than 3 mL/min, no more than 2 mL/min, no more than 1 mL/min, no more than 0.5 ml/min or no more than 0.1 ml/min, or
wherein a flow rate of one or both of the acidic elution buffer solution and the wash buffer solution through the chromatography resin medium during the elution or washing steps comprises a flow rate per minute of approximately an equal volume or less of the acidic elution buffer solution or wash buffer solution per volume of chromatography resin medium.

38.-42. (canceled)

43. The method according to claim 1, wherein a direction of flow of the elution acidic buffer solution in an elution step is opposite to a direction of flow of the cell lysate through the chromatography resin medium in the contacting step, whereby an increased quantity of AAV particles are eluted from the chromatography resin medium compared to when the directions of flow in the elution and providing steps are the same.

44. The method according to claim 1, wherein the method further comprises a step of capturing the at least one or more elution volume fractions,
wherein the method further comprises a step of sterilizing the at least one or more elution volume fractions containing the purified one or more AAV vector particles, and/or
wherein the method further comprises the step of submitting the at least one or more elution volume fractions to a buffer exchange.

45.-137. (canceled)

138. A method of high efficiency purification of adeno-associated virus (AAV) particles comprising the steps of:
a. transfecting at least one cell with plasmid DNA comprising an AAV vector genome, one or more AAV capsid genes and one or more transacting helper genes;
b. providing a cell lysate that comprises one or more AAV particles by lysing the transfected at least one cell in situ;
c. contacting the cell lysate comprising the one or more AAV particles with a volume of chromatography resin medium comprising at least one ligand possessing a pan-AAV affinity, wherein said contacting induces binding of the one or more AAV particles to the chromatography resin medium; and
d. eluting the bound one or more AAV particles from the chromatography resin medium using a volume of an acidic elution buffer solution to provide one or more eluted volume fractions containing the one or more AAV particles;
whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, wherein the method does not require modifications contingent on the AAV serotype being purified to obtain said efficiency and wherein at least one of the following is true:
i. at least one of the method steps is carried out in the presence of a non ionic surfactant,
ii. the contacting is performed at a temperature of between approximately 20° C. to approximately 28° C.,
iii. both the cell lysate and the chromatography resin medium are allowed to come to a temperature between approximately 20° C. to approximately 28° C. previous to said contacting, or
iv. the method further comprises a step of regenerating the chromatography resin medium comprising the at least one ligand possessing a pan-AAV affinity by contacting the medium with at least one acidic component for a predetermined amount of time.

139.-184. (canceled)

185. A method of high efficiency purification of adeno-associated virus (AAV) particles comprising the steps of:
e. seeding HEK293T cells onto a substrate which holds a Dulbecco's modified eagle medium (DMEM) containing 10% fetal bovine serum (FBS) and 1% mixture of penicillin G and streptomycin (penstrep) and allowing expansion of the cells until approximately 80% confluency is obtained;
f. transfecting the HEK293T cells by contacting said cells with a composition that comprises DMEM, polyethylenimine, penstrep, and plasmid DNA comprising an AAV vector genome, one or more AAV capsid genes and one or more trans-acting helper genes;
g. providing a clarified cell lysate that comprises one or more AAV particles by lysing the transfected HEK293T cells in situ utilizing TRITON-X 100, RNAse A, Turbonuclease and PLURONIC F68, subjecting the lysate to centrifugation at 4,000 g or higher and subsequently filtering a supernatant thus obtained by use of a 0.45 μm cellulose acetate/polyethersulfone membrane filter system;
h. contacting the clarified cell lysate comprising the one or more AAV particles with a 1 mL volume of POROS CAPTURESELECT AAVX chromatography resin medium, wherein the contacting duration comprises no less than 1 minute, wherein the contacting is performed at a temperature of between approximately 21° C. to approximately 25° C. and/or both the cell lysate and the chromatography resin medium are allowed to come to a temperature between approximately 21° C. to approximately 25° C. previous to said contacting, and wherein said contacting induces binding of the one or more AAV particles to the chromatography resin medium;
i. eluting the bound one or more AAV particles from the chromatography resin medium using a volume of a filtered acidic elution buffer solution having a pH between approximately 2.0 and approximately 2.5, to provide one or more eluted volume fractions containing the one or more AAV particles, wherein the filtered acidic elution buffer solution comprises 0.2M glycine and 0.01 v/v % PLURONIC F68 and wherein a flow direction of the acidic elution buffer solution through the chromatography resin medium is opposite a flow direction of the clarified cell lysate through the chromatography resin during the contacting step;
j. optionally sterilizing the at least one or more elution volume fractions containing the purified one or more AAV particles utilizing a 0.2 μm polyethersulfone syringe filter;
k. subjecting the one or more elution volume fractions containing the purified one or more AAV particles to buffer exchange using either AMICON UTRACEL 15 or AMICON Stirred Cell concentrators, wherein if a 50 or 100 kDA AMICON ULTRA 15 Centrifugal Filter device is used for buffer exchange, less than 1×10¹³ vg of AAV are subjected to buffer exchange therein to reduce sedimentation and loss; and
l. regenerating the chromatography resin medium by contacting the chromatography resin medium with 0.1M phosphoric acid for at least 15 minutes, followed or preceded by contacting the chromatography resin medium with 6M guanidine for at least 15 minutes; whereby purified AAV particles are obtained with an overall efficiency of at least 65% regardless of the serotype of the AAV purified, wherein the purity, yield and bioactivity of the AAV particles purified thereby are comparable to the purity, yield and bioactivity of iodixanol purified AAV particles, wherein any plastic surface which comes into contact with the AAV particles during the method is first coated with a composition comprising PLURONIC F68, and wherein the method does not require modifications contingent on the AAV serotype being purified to obtain said efficiency.


SEQUENCE LISTING (26 sequences)

SEQ ID NO: 1 - DNA, 20 bp, Artificial
  primer
agcaaagaccccaacgagaa

SEQ ID NO: 2 - DNA, 15 bp, Artificial
  primer
ggcggcggtcacgaa

SEQ ID NO: 3 - DNA, 22 bp, Artificial
  primer
cgcgatcacatggtcctgctgg

SEQ ID NO: 4 - DNA, 23 bp, Artificial
  primer
gttttcatcactgttcatgatgc

SEQ ID NO: 5 - DNA, 22 bp, Artificial
  primer
tcatgggcattactattcctac

SEQ ID NO: 6 - DNA, 35 bp, Artificial
  probe
aggaccagcttctctgcattatcatcgttgaagat

SEQ ID NO: 7 - DNA, 23 bp, Artificial
  RPII RNA
aatcaatgcaggttttggcgatg

SEQ ID NO: 8 - protein, 743 aa, Artificial
  AAV
MSLISDAIPDWLERLVKKGVNAAADFYHLESGPPRPKANQQTQESLEKDDSRGLVFPGYNYLGPFNGLDKGEPVNEADAAALEHDKAYDLEIKDGHNPYFEYNEADRRFQERLKDDTSFGGNLGKAIFQAKKRVLEPFGLVEDSKTAPTGDKRKGEDEPRLPDTSSQTPKKNKKPRKERPSGGAEDPGEGTSSNAGAAAPASSVGSSIMAEGGGGPVGDAGQGADGVGNSSGNWHCDSQWLENGVVTRTTRTWVLPSYNNHLYKRIQGPSGGDNNNKFFGFSTPWGYFDYNRFHCHFSPRDWQRLINNNWGIRPKAMRFRLFNIQVKEVTVQDFNTTIGNNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQAGSSGRALHYSRATKTNMAAQYRNWLPGPFFRDQQIFTGASNITKNNVFSVWEKGKQWELDNRTNLMQPGPAAATTFSGEPDRQAMQNTLAFSRTVYDQTTATTDRNQILITNEDEIRPTNSVGIDAWGAVPTNNQSIVTPGTRAAVNNQGALPGMVWQNRDIYPTGTHLAKIPDTDNHFHPSPLIGRFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 9 - protein, 311 aa, Artificial
  AAV
MAADGYLDNLSEIREWWAKPAQHNALKGAQQLADLKHAEERVLLEAAKTAPKPVQSQESAGIGSGAQAKLNFQTGDTESVPDPQPIGEPPAPGLTSAANNESGDRITSATQSNSTSGSSDAYYFFRLNKDNNGVKATSHCLVFMLDGSQGSEQQSENSYSSIYYLKTINGSQNQQTKFVGPSVGYISYQRVSTVTQNNNSEFAPGASSANGNSNMSHKEDRFFPLSGSIGKQGTGRDNVADKVMEKTPATESYQAHAQAQAQTGWQIDVLQPIWHGNMGMLDPTANKDLNTVSEQNYYYKSNNVENTEVRN

SEQ ID NO: 10 - protein, 317 aa, Artificial
  AAV
MAADGYLDNLSEIREWWAKPAQHNALKGAQQLADLKHAEERVLLEAAKTAPKPVQSQESAGIGSGAQAKLNFQTGDTESVPDPQPIGEPPAPGLTSAANNESGDRITSATQSNSTSGSSDAYYFFRLNKDNNGVKATSHCLVFMLDGSQGSEQQSENSYSSIYYLRTINGSQNQQTKFVGPSVGYISYQRVSTVTQNNNSEFAPGASSANGNSNMSHKEDRFFPLSGSIGKQGTGRDNVADKVMEKTPATESYQAHAQLAVPFKAQAQTGWQIDVLQPIWHGNMGMLDPTANKDLNTVSEQNYYYKSNNVENTEVRN

SEQ ID NO: 11 - protein, 324 aa, Artificial
  AAV
MAADGYLDTLSEIRQWWKKPPPAERHKLKRQLDSDLKHAEERVLEPVKTAPKPVHSVESGGAGQQARKLNFQTGDADSVPDPQPLGQPPAPGLTNTTSAMANNETMGDRTTSATQSSQSASDHYYFFRLNKQNGTATSEHQCLVFMVLNGSQGSEQTSSYSSIYYLRTNTPSGTTTQSRQFQGASDIRDSCYQRVSKTSADNNNSEYSTGATKYHNGDSVNMSHKDDEKFFPQSGVIGKQGSEKTNVIEKVMDETPATEQYSSLRGNLALGETTRPARQAATDTVDVLQPIWHGMGLLTSAFTVSEQNYYNKSINVDTDTNVRN

SEQ ID NO: 12 - protein, 314 aa, Artificial
  AAV
MAADGYLDTLSEIRQWWKKPPPAERHKLKRQLDSDLKHAEERVLEPVKTAPKPVHSVESGGAGQQARKLNFQTGDADSVPDPQPLGQPPAPGLTNTTSAMANNETMGDRITSATQSSQSASDHYYFFRLNKQNGTATSEHQCLVFMVLNGSQGSEQTSSYSSIYYLRTNTPSGTTTQSRQFQGASDIRDSCYQRVSKTSADNNNSEYSTGATKYHNGDSVNMSHKDDEKFFPQSGVIGKQGSEKTNVIEKVMDETPATEQYSSLRGNRQAATDTVDVLQPIWHGMGLLTSAFTVSEQNYYNKSVNVDTDTNVRN

SEQ ID NO: 13 - protein, 313 aa, Artificial
  AAV
MAADGYLDTLSEIRQWWKKPPPAERHKLKRQLDSDLKHAEERVLEPVKTAPKPVHSVESGGAGQQARKLNFQTGDADSVPDPQPLGQPPAPGLTNTTSAMANNETMGDRITSATQSSQSASDHYYFFRLNKQNGTATSEHQCLVFMVLNGSQGSEQTSSYSSIYYLRTNTPSGTTTQSRQFQGASDIRDSCYQRVSKTSADNNNSEYSTGATKYHNGDSVNMSHKDDEKFFPQSGVIGKQGSEKTNVIEKVMDETPATEQYSSLGNTQAATDTVDVLQPIWHGMGLLTSAFTVSEQNYYNKSVNVDTDTNVRN

SEQ ID NO: 14 - protein, 309 aa, Artificial
  AAV
MAADGYLDNLSEIREWWDKPAKKNGLKAQQLADLRHAEQERVLEGAKTAPAKPVPSQRSSTGIGGQQARKLNFQTGDSESVPDPQPLGEPPAPGTVAAMANNEATGDRITSATQSSETASTDTYYFFKLKTNGVAISSEHQCLVFMLNGSQSGSEQSSSYSSIYYLARTQSNPGGTAGNEQFYQGGPSTEAKCQRVSKTLDQNNNSNFATGATKYHNGNSVNVMHKDDDRFFPSSGVIGKGATNKTLENVMEPATEEYISSLAANAAQTQVVLQPIWHGNMGLLPVTPFTVSEQNYEKQTGVDDSQVRN

SEQ ID NO: 15 - protein, 308 aa, Artificial
  AAV
MAADGYLDNLSEIREWWDKPAKKGLKAQQLADLRHAEQERVLEGAKTAPKPIGSSTGIGGQQAKKLNFQTGDSESVPDPQPLGEPPAPGTAAMANNEATGDRITSATQSSSSATDHYYFFKLKLTNGVASSEHQCLVFMLDNGSQSGSEQSSSYSSIYYLARTQSTTGSTEQFHQGPNTESKCYQRLSKNLDFNNNSNFATAATKYHNGNSTNIPMNKDDDQFFPINGVVGKGAANKTLENVMSEKTPATEEYVSSLSTAGPQSQTISVLQPIWHGNMGLLPVTPFTVSEQNYYAKSNNVENPDVTRN

SEQ ID NO: 16 - protein, 319 aa, Artificial
  AAV
MAADGYLDNLSEIREWWDKPAKKGLKAQQLADLRHAEQERVLEGAKTAPKPVQSQESGIGTGQQAKLNFQTGDSESVPDPQPLGEPPTPAAPTTSAMANNEATGDRITSATQSSATASDHYYFFRLNKTNGVASSEHQCLVFMLNGSQGSEQTSSYSSIYYLNRTQNQSGSAQNKDLFGSPAGSVPKCYQRVSKTKTDNNNSNFTTGASKYNNGESIINTMSHKDDKDKFFPSGVMIGKESAGASNALDNVMDEKAPATERFTAVLSSDPATGDHVMDVLQPIWHGMGLLPAESATFTVSEQNVYYAKSNVDTDNNLTR

SEQ ID NO: 17 - protein, 320 aa, Artificial
  AAV
MAADGYLDNLSEIREWWDKPAKKGLKAQQLADLRHAEQERVLEGAKTAPKPVQSQESGIGTGQQAKLNFQTGDSESVPDPQPLGEPPTPAAPTTSAMANNEATGDRITSATQSSATASDHYYFFRLNKTNGVASSEHQCLVFMLNGSQGSEQTSESYSSIYYLNRTQNQSGSAQNKDLFGSPAGSVPKCYQRVSKTKTDNNNSNFTTGASKYNNGESIINTMSHKDDDKFFPSGVMIGKESAGASNALDNVMDEKAPATERFTAVFSSDPATGDHAMDVLQPIWHGMGLNLPAESATFTVSEQNVYYAKSNVDTDNNLTR

SEQ ID NO: 18 - protein, 313 aa, Artificial
  AAV
MAADGYLDNLSEIREWWAKPVQHNRLKGQQLADLKHAEQERVILEAAKTAPKGAVDQSQESGVGSGQARKLNFQTGDSESVPDPQPLGEPPAPTLNTSAMANNEGDRITSATQSSQSASDHYYFFKLSKRGQNGTATSEHQCLVFMVLNGSQGSEQQSSYSSIYYLNRTQGTTSGTTNQSRLFQGPQSSLACYQRLSKTNDNNNSNFPTAASKYHNGDSVNMSHKDDEKFFPHGNIGKEGTTASNAELDNVMDETPATEQYTANLSNAPTGTHDVLQPIWHGMGLMPTSPFTVSEQNYYNKSVNVDTDTNVRN

SEQ ID NO: 19 - protein, 319 aa, Artificial
  AAV
MAADGYLDNLSEIREWWAKPAKKGLKAQQLQADLRHAEQERVLEGAKTAPKPVPSQRSSTGIGGQQARKLNFQTGDSESVPDPQPLGEPPAPGPNTAAMANNESTGDRITSATQSNGTSGATDTYYFFRLSKQNEGTKAITSEHQCLVFMLNGSQGSEQQSYSSIYYLRTQTTGGTANTQTGFQGGPNTNAKCYQRVSTTGQNNNSNFATATKYHNGNSANIMHKDDERFFPSNGIIGKQNAARDNAYSDVMLSEKTPATEEYIADLQQNAPQIGTSVLQPIWHGNMGLLDPTNQSLNTVSEQNYYYKSTSVDNTEVRN

SEQ ID NO: 20 - protein, 319 aa, Artificial; identical to SEQ ID NO: 19
  AAV

SEQ ID NO: 21 - protein, 314 aa, Artificial
  AAV
MAADGYLDNLSEIREWWDKPAKKGLKAQQLADLRHAEQERVLEGAKTAPKPVPSQRSSTGIGGQQAKLNFQTGDSESVPDPQPIGEPPGPGLGTAAMANNESTGDRITSATQSNGTSGSTDTYYFFRLNKQNEGTKAITSEHQCLVFMLNGSQGSEQSQSYSSIYYLRTQSTGGTAGTQQLFQGPNSAKCYQRVSTLQNNNSNFATGATKYHNGDSVNVMHKDDERFFPSSGVMGKQGAGKDNVYSSVMLSEKTPATEQYVADLQQNAAPIVGSVLQPIWHGNMGLLDPTSQLTVSEQNYYYKSTNVDNTDTRN

SEQ ID NO: 22 - protein, 316 aa, Artificial
  AAV
MAADGYLDNLSEIREWWDKPAKKGLKAQQLADLRHAEQERVLEGAKTAPKPVPSQRSSTGIGGQQARKLNFQTGDSESVPDPQPIGEPPAPGTAAMANNESTGDRITSATQSNGTSGSTDTYYFPFRLSKQNEGTKAITSEHQCLVFMLNGSQGSEQSSPSYSSIYYLRTQSTGGTAGTQQLFQGPSSACYQRVSTLQNNNSNFATGATKYHNGDSVNVMNKDDDRFFPSSGIMGKQGAGKDNVYSNVMLSEKTPATEQYVADLQQDAPIVGSVLQPIWHGNMGLVLDPTANQLNTVSEQNYYYKSTNVDNTEVRN

SEQ ID NO: 23 - protein, 315 aa, Artificial
  AAV
MAADGYLDNLSEIREWWDKPAKKGLKAQQLADLRHAEQERVLEGAKTAPKPVQSQESGIGTGQQAKLNFQTGDSESVPDPQPLGEPPAPGLPNTSAMANNETGDRITSATQSNGTSGSTDTYYFFRLNKTNEGTKATSEHQCLVFMVLNGSQLGSEQQSSYSSIYYLVRTQTTGTGGTQTAFQGPSSNAVCYQRVSTTNQNNNSNFATGAAKFKNGDSNVMSHKDDDDRFFPSSGVIGKQGAGNDGVYSVDEKAPATEEYAIAANQAQTGLHVIVLQPIWHGNMGLLDPLNQLNTVSEQNYYYKSTNVDNTEVRN

SEQ ID NO: 24 - protein, 303 aa, Artificial
  AAV
MAADGYLDNLSEIREWWDKPAKKGLKAQQLADLRHAEQERVLEGAKTAPKPVQSQESGIGGQQARKLNFQTGDSESVPDPQPLGEPPAPGNTAAMANNEATGDRITSATQSSQSGSTDTYYFFKLNKTNGTATSEHQCLVFMLNGSQGSEQQSSYSSIYYLRTQTTSGTAGNTQFQGPSSNAKCYQRVSKTTNQNNNSNFATGATKYHNGDSVNMHKDDDKFFPSGVIGKQGAGNSNVLDNVMEKTPATEEYTALANAPATGTSDVLQPIWHGMGLLPTSPFTVSEQNYYNKSTNVDDTNVRN

SEQ ID NO: 25 - protein, 331 aa, Artificial
  AAV
MAADGYLDNLSEIREWWDKPAKKGLKAQQLADLRHAEQERVLEGAKTAPKPLESQESGIGGQAKLNFEEDTGAGDGPPEGSDTSAMSDIERAAPNAVSADTSGKTTSTLLGTTSSTYNFLVKITSNGEVAISSEMDAGQSLNVFMVGIVTGENQNQTNEQMANKYSSLHLQTTSGETLNQGNATTFGKIRSGDFFYRKCVKQRFSKTQNYKIPASGGNALLKYDTHYTNWSNIAPMAGPSDGFSNAQLIFPGPSTGNTSANLFSEAAPRDTMFQIADNATAPITGNTAMVYQPIWHAGGLATTARDTVAQEIERVYQSSMLWPDTKTVSNH

SEQ ID NO: 26 - protein, 340 aa, Artificial
  AAV
MTDGYLDNLSEREWWAQPAKHNALKGAQQLADLKHAEQQGRVLQAGETAPKPLIESQQSTGIGGQAKKLVFEDTGAGDGPPEGSTSGAMDDERAAAAAVEGADTSGHTTSTLGESLQSTYNFMVKITSNGEVAISSEMDAGQSLNVFMVGVTGNTSQQQTNEQISKYSSIGLQTTTGTTLNAGTATTNFTKLRPFSNFKKSIKQGFSKTNQNYKIPATGSDLIKYETHSTGWSALTPMAGPADSKFSNSQLIFAGPKQNGNTAVPGTLIFSELAAATDTMNLGGDNSNLPVDRLTALVYQPIWHGGLATSSTPNTVSQDIQRVYQQNSLLWPDAAKTAHH